(19) United States
(12) Patent Application Publication
(10) Pub. No.: US 2006/0178400 A1
(43) Pub. Date: Aug. 10, 2006

(54) Novel acetyl-CoA carboxylase (ACC) inhibitors and their use in diabetes, obesity and metabolic syndrome

(76) Inventors: Bruce A. Beutel, Lake Forest, IL (US); Heidi S. Camp, Winnetka, IL (US); Richard F. Clark, Gurnee, IL (US); Yu-Gui Gu, Libertyville, IL (US); Todd M. Hansen, Grayslake, IL (US); Robert F. Keyes, Pleasant Prairie, WI (US); Qun Li, Libertyville, IL (US); Gang Liu, Libertyville, IL (US); Hing L. Sham, Vernon Hills, IL (US); Xiaojun Wang, Libertyville, IL (US); Moshe Weitzberg, Highland Park, IL (US); Zhili Xin, Lake Bluff, IL (US); Xiangdong Xu, Buffalo Grove, IL (US); Tianyuan Zhang, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/268,698

(22) Filed: Nov. 7, 2005

Related U.S. Application Data
(60) Provisional application No. 60/625,686, filed on Nov. 5, 2004.

(51) Int. Cl. C07D 211/68; A61K 31/445

(52) U.S. Cl. 514/317; 514/340; 546/194; 546/268.1

(57) ABSTRACT

The present invention relates to compounds of formula (I), 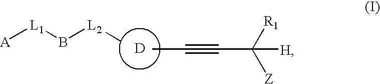 which inhibit acetyl-CoA carboxylase (ACC) and are useful for the prevention or treatment of metabolic syndrome, type II diabetes, obesity, atherosclerosis and cardiovascular diseases in humans.

This application claims priority from U.S. Provisional Patent Application Ser. No. 60/625,686, filed Nov. 5, 2004.

### FIELD OF THE INVENTION

The present invention relates to compounds which inhibit acetyl-CoA carboxylase (ACC) and are useful for the prevention or treatment of metabolic syndrome, type 2 diabetes, obesity, atherosclerosis and cardiovascular diseases in humans.

### BACKGROUND

The incidence of type 2 diabetes has dramatically increased over the past decade. This epidemic is largely attributed to proliferation of key risk factors, which include a sedentary lifestyle, a high fat diet, obesity and the demographic shift to a more aged population. There is ample evidence to indicate that increased abdominal obesity and physical inactivity contribute significantly to the development of type 2 diabetes (Turkoglu C, Duman B S, Gunay D, Cagatay P, Ozcan R, Buyukdevrim A S: Effect of abdominal obesity on insulin resistance and the components of the metabolic syndrome: evidence supporting obesity as the central feature. Obes Surg 2003; 13: 699-705. Steyn N P, Mann J, Bennett P H, Temple N, Zimmet P, Tuomilehto J, Lindstrom J, Louheranta A: Diet, nutrition and the prevention of type 2 diabetes. Public Health Nutr 2004; 7: 147-65).

At the cellular level, an increase in ectopic fat storage in nonadipose tissues such as in muscle, liver and pancreas is a strong predictor of the development of insulin resistance and type 2 diabeties (Hulver M W, Berggren J R, Cortright R N, Dudek R W, Thompson R P, Pories W J, MacDonald K G, Cline G W, Shulman G I, Dohm G L, Houmard J A: Skeletal muscle lipid metabolism with obesity. Am J Physiol Endocrinol Metab 2003; 284: E741-7. Sinha R, Dufour S, Petersen K F, LeBon V, Enoksson S, Ma Y Z, Savoye M, Rothman D L, Shulman G I, Caprio S: Assessment of skeletal muscle triglyceride content by ¹H nuclear magnetic resonance spectroscopy in lean and obese adolescents: relationships to insulin sensitivity, total body fat, and central adiposity. Diabetes 2002; 51: 1022-7). The precise mechanism of how increased intracellular lipid content exacerbates whole body insulin sensitivity is unclear at present but it has been postulated that increased long chain fatty acyl-CoAs, ceramide or diacylglycerol, whose contents are proportional to the accumulation of intramyocellular triglyceride, antagonizes metabolic actions of insulin, reduces muscle glucose uptake and inhibits hepatic glucose production (Sinha R, Dufour S, Petersen K F, LeBon V, Enoksson S, Ma Y Z, Savoye M, Rothman D L, Shulman G I, Caprio S: Assessment of skeletal muscle triglyceride content by ¹H nuclear magnetic resonance spectroscopy in lean and obese adolescents: relationships to insulin sensitivity, total body fat, and central adiposity. Diabetes 2002; 51: 1022-7. Friedman J: Fat in all the wrong places. Nature 2002; 415: 268-9). As muscle is the primary site of metabolic action of insulin, the development of muscle insulin resistance along with liver insulin resistance are thus inherently linked to the development of whole body insulin resistance.

In order to increase muscle and liver fat oxidation and thus limit the concentration of LCFACoA's we aim to inhibit the activity of Acetyl CoA Carboxylase (ACC), which catalyzes the production of malonyl-CoA from acetyl-CoA. Malonyl-CoA is an intermediate substrate that plays an important role in the overall fatty acid metabolism: Malonyl-CoA is utilized by fatty acid synthase for de novo lipogenesis, and also acts as a potent allosteric inhibitor of carnitine palmitoyltransferase 1 (CPT1), a mitochondrial membrane protein that shuttles long chain fatty acyl CoAs into the mitochondrial where they are oxidized (Ruderman N, Prentki M: AMP kinase and malonyl-CoA: targets for therapy of the metabolic syndrome. Nat Rev Drug Discov 2004; 3: 340-51). A small molecule inhibitor of ACC would thus limit de novo lipid synthesis, de-inhibit CPT1 and subsequently increase fat oxidation.

In rodents and in humans, there are two known isoforms of ACC that are encoded by distinct genes and share approximately 70% amino acids identity. ACC1, which encodes a 265 KD protein, is highly expressed in the cytosol of lipogenic tissues such as liver and adipose, whereas 280 KD ACC2 protein is preferentially expressed in oxidative tissues, skeletal muscle and heart (Mao J, Chirala S S, Wakil S J: Human acetyl-CoA carboxylase 1 gene: presence of three promoters and heterogeneity at the 5′-untranslated mRNA region. Proc Natl Acad Sci USA 2003; 100: 7515-20. Abu-Elheiga L, Almarza-Ortega D B, Baldini A, Wakil S J: Human acetyl-CoA carboxylase 2. Molecular cloning, characterization, chromosomal mapping, and evidence for two isoforms. J Biol Chem 1997; 272: 10669-77). ACC2 has a unique 114 amino acid N-terminus with a putative transmembrane domain (TM), which is thought to be responsible for mitochondrial targeting (Abu-Elheiga L, Brinkley W R, Zhong L, Chirala S S, Woldegiorgis G, Wakil S J: The subcellular localization of acetyl-CoA carboxylase 2. Proc Natl Acad Sci USA 2000; 97: 1444-9). Based on tissue distribution and subcellular localization of these two isoforms, the current hypothesis is that a distinct pool of Malonyl-CoA produced by ACC1 is preferentially converted into fatty acids by fatty acid synthase, whereas another pool of Malonyl-CoA synthesized primarily by ACC2, presumed localized in near mitochondria, may be involved in the inhibition of CPT1 (Abu-Elheiga L, Brinkley W R, Zhong L, Chirala S S, Woldegiorgis G, Wakil S J: The subcellular localization of acetyl-CoA carboxylase 2. Proc Natl Acad Sci USA 2000; 97: 1444-9). Therefore, ACC1 inhibition will reduce fatty acid synthesis and therefore may be beneficial for use in treating diseases such as metabolic syndrome.

Genetic studies have demonstrated that ACC2 knockout mice are healthy and fertile with a favorable metabolic phenotype, increased fatty acid oxidation, increased thermogenesis, reduced hepatic TG content and subsequent decrease in body weight despite increase in food intake compared to their littermates (Abu-Elheiga L, Matzuk M M, Abo-Hashema K A, Wakil S J: Continuous fatty acid oxidation and reduced fat storage in mice lacking acetyl-CoA carboxylase 2. Science 2001; 291: 2613-6). In addition, these mice are resistant against high fat diet-induced obesity and insulin resistance (Abu-Elheiga L, Oh W, Kordari P, Wakil S J: Acetyl-CoA carboxylase 2 mutant mice are protected against obesity and diabetes induced by high-fat/high-carbohydrate diets. Proc Natl Acad Sci USA 2003; 100: 10207-12). Also, recently it was demonstrated that the effects of leptin and adiponectin, cytokines secreted from adipose tissue, to increase fatty acid oxidation are at least due in part to the inhibition of ACC in liver and skeletal muscle (Yamauchi T, Kamon J, Waki H, Terauchi Y, Kubota N, Hara K, Mori Y, Ide T, Murakami K, Tsuboyama-Kasaoka N, Ezaki O, Akanuma Y, Gavrilova O, Vinson C, Reitman M L, Kagechika H, Shudo K, Yoda M, Nakano Y, Tobe K, Nagai R, Kimura S, Tomita M, Froguel P, Kadowaki T: The fat-derived hormone adiponectin reverses insulin resistance associated with both lipoatrophy and obesity. Nat Med 2001; 7: 941-6). Taken together these data support that the discovery of small molecular inhibitors of ACC2 may provide a favorable metabolic profile against obesity induced type 2 diabetic patients. Furthermore, the dual inhibition of ACC1 and ACC2 may provide the prfile needed to demonstrate benefit for patients exhibiting conditions of metabolic syndrome.

### BRIEF DESCRIPTION OF THE DRAWINGS

FIG. 1: As shown in FIG. 1, RER is unchanged from vehicle prior to meal challenge. Upon meal challenge RER is increased with vehicle and Compound I, but significantly less, indicating an increase in whole body fat oxidation and decrease in carbohydrate oxidation with Compound I.

FIG. 2: As shown in FIG. 2, RER is unchanged from vehicle prior to meal challenge. Upon meal challenge RER is increased with vehicle and Compound II, but significantly less, and in a dose-dependent manner, indicating an increase in whole body fat oxidation and decrease in carbohydrate oxidation with Compound II.

### SUMMARY OF THE INVENTION

The present invention is directed to compounds of formula (I), 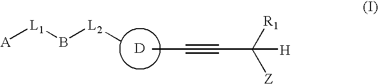
or therapeutically suitable salt, ester or prodrug, thereof, wherein

A is selected from the group consisting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl;

B is selected from the group consisting of an aryl ring and a heteroaryl ring;

D is selected from the group consisting of an aryl ring and a heteroaryl ring;

L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;

L₂ is selected from the group consisting of —C(R_{d}Rₑ)—, —(CH₂)ₙ—, —NH—, —O—, and —S—;

n is 1, 2 or 3;

Z is a member selected from the group consisting of alkoxy, hydroxy, hydroxyalkyl, R_{g}—O— and Rⱼ—NH—;

R₁ is hydrogen, C₁₋₆haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}—N═;

R_{c} is selected from the group consisting of hydrogen, alkyl, aryl, haloalkyl, and heteroaryl;

R_{d} is selected from the group consisting of alkyl, haloalkyl, hydroxy and halo;

Rₑ is selected from the group consisting of hydrogen, alkyl, haloalkyl, hydroxy and halo, or R_{d} and Rₑ taken together with the atom to which they are attached form oxo;

R_{f} is selected from the group consisting of alkoxy, aryloxy, heteroaryloxy and hydroxy;

R_{g} is H₂N—C(O)— or C₁₋₆ alkylHN—C—(O)—; and

Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Accordingly, the present invention is directed toward compounds of formula (I), which inhibit acetyl-CoA carboxylase (ACC). As inhibitors of acetyl-CoA carboxylase (ACC), the compounds of the present invention may be useful for the treatment or prevention of diseases such as metabolic syndrome, type II diabetes, obesity, atherosclerosis and cardiovascular diseases in humans The present invention is also directed to a method of administering a therapeutically effective amount of a compound of formula (I). In another embodiment of the present invention, there is provided a method for treating metabolic syndrome, type II diabetes, obesity, atherosclerosis and cardiovascular diseases in humans comprising administering a therapeutically effective amount of a compound of formula (I).

According to still another embodiment, the present invention is directed to a pharmaceutical composition comprising a therapeutically effective amount of a compound of formula (I) in combination with a pharmaceutically suitable carrier.

### DETAILED DESCRIPTION OF THE INVENTION

Accordingly, the principle embodiment of the present invention is directed toward a compound of formula (I), 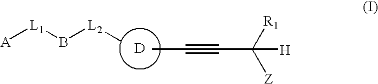
or therapeutically suitable salt, ester or prodrug, thereof, wherein

A is selected from the group consisting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl;

B is selected from the group consisting of an aryl ring and a heteroaryl ring;

D is selected from the group consisting of an aryl ring and a heteroaryl ring;

L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;

L₂ is selected from the group consisting of —C(R_{d}Rₑ)—, —(CH₂)ₙ—, —NH—, —O—, and —S—;

n is 1, 2 or 3;

Z is a member selected from the group consisting of alkoxy, hydroxy, hydroxyalkyl, R_{g}—O— and Rⱼ—NH—;

R₁ is hydrogen, C₁₋₆ haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}N═;

R_{c} is selected from the group consisting of hydrogen, alkyl, aryl, haloalkyl, and heteroaryl;

R_{d} is selected from the group consisting of alkyl, haloalkyl, hydroxy and halo;

Rₑ is selected from the group consisting of hydrogen, alkyl, haloalkyl, hydroxy and halo, or R_{d} and Rₑ taken together with the atom to which they are attached form oxo;

R_{f} is selected from the group consisting of alkoxy, aryloxy, heteroaryloxy and hydroxy;

R_{g} is H₂N—C(O)— or C₁₋₆ alkylHN—C—(O)—; and

Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of alkenyl, alkoxyalkyl and alkyl; B is selected from the group consisting of an aryl ring and a heteroaryl ring; D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₆ haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{b} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₆ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of alkenyl, alkoxyalkyl and alkyl; B is selected from the group consisting of an aryl ring and a heteroaryl ring; D is selected from the group consisting of an aryl ring and a heteroaryl ring; L₁ is absent; L₂ is selected from the group consisting of —C(R_{d}Rₑ)—, —(CH₂)ₙ—, —NH—, —O—, and —S—; n is 1, 2 or 3; Z is a member selected from the group consisting of hydroxy, hydroxyalkyl, R_{g}—O— and Rⱼ—NH—; R₁ is hydrogen, C₁₋₆ haloalkyl or C₁₋₃ alkyl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of alkenyl, alkoxyalkyl and alkyl; B is selected from the group consisting of an aryl ring and a heteroaryl ring; D is selected from the group consisting of an aryl ring and a heteroaryl ring; L₁ is selected from the group consisting of —NR_{c}—, —NR_{c}CH₂—, —O—, and —S—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of alkenyl, alkoxyalkyl and alkyl; B is selected from the group consisting of an aryl ring and a heteroaryl ring; D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; R_{c} is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of alkenyl, alkoxyalkyl and alkyl; B is selected from the group consisting of an aryl ring and a heteroaryl ring; D is selected from the group consisting of an aryl ring and a heteroaryl ring; L₁ is —C(RₐR_{b})—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O—and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl and hydroxy or Rₐ and R_{b} taken together with the atom to which they are attached form R_{f}N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of aryl and arylalkyl, B is selected from the group consisting of an aryl ring and a heteroaryl ring; D is selected from the group consisting of an aryl ring and a heteroaryl ring; L₁ is absent; L₂ is selected from the group consisting of —C(R_{d}Rₑ)—, —(CH₂)ₙ—, —NH—, —O—, and —S—; n is 1, 2 or 3; Z is a member selected from the group consisting of hydroxy, hydroxyalkyl, R_{g}—O— and Rⱼ—NH—; R₁ is hydrogen, C₁₋₃ haloalkyl or C₁₋₃ alkyl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of aryl, arylalkyl, heteroaryl and heteroarylalkyl; B is selected from the group consisting of an aryl ring and a heteroaryl ring; D is selected from the group consisting of an aryl ring and a heteroaryl ring; L₁ is selected from the group consisting of —NR_{c}—, —NR_{c}CH₂—, —O—, and —S—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of aryl, arylalkyl, heteroaryl and heteroarylalkyl; B is selected from the group consisting of an aryl ring and a heteroaryl ring; D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of aryl, arylalkyl, heteroaryl and heteroarylalkyl; B is selected from the group consisting of an aryl ring and a heteroaryl ring; D is selected from the group consisting of an aryl ring and a heteroaryl ring; L₁ is —C(RₐR_{b})—; L₂ is selected from the group consisting of —C(R_{d}Rₑ)—, —CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl and hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂—N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of cycloalkyl, cycloalkylalkyl, heterocycle and heterocyclealkyl; B is selected from the group consisting of an aryl ring and a heteroaryl ring; D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl, hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of cycloalkyl, cycloalkylalkyl, heterocycle and heterocyclealkyl; B is selected from the group consisting of an aryl ring and a heteroaryl ring; D is selected from the group consisting of an aryl ring and a heteroaryl ring; L₁ is absent; L₂ is selected from the group consisting of —C(R_{d}Rₑ)—, —(CH₂)ₙ—, —NH—, —O—, and —S—; n is 1, 2 or 3; Z is a member selected from the group consisting of hydroxy, hydroxyalkyl, R_{g}—O— and Rⱼ—NH—; R₁ is hydrogen, C₁₋₃ haloalkyl or C₁₋₃ alkyl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of cycloalkyl, cycloalkylalkyl, heterocycle and heterocyclealkyl; B is selected from the group consisting of an aryl ring and a heteroaryl ring; D is selected from the group consisting of an aryl ring and a heteroaryl ring; L₁ is selected from the group consisting of —NR_{c}—, —NR_{c}CH₂—, —O—, and —S—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of cycloalkyl, cycloalkylalkyl, heterocycle and heterocyclealkyl; B is selected from the group consisting of an aryl ring and a heteroaryl ring; D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of cycloalkyl, cycloalkylalkyl, heterocycle and heterocyclealkyl; B is selected from the group consisting of an aryl ring and a heteroaryl ring; D is selected from the group consisting of an aryl ring and a heteroaryl ring; L₁ is —C(RₐR_{b})—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O—and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl, hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of heteroaryl and heteroarylalkyl, B is selected from the group consisting of an aryl ring and a heteroaryl ring; D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl and hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of heteroaryl and heteroarylalkyl, B is selected from the group consisting of an aryl ring and a heteroaryl ring; D is selected from the group consisting of an aryl ring and a heteroaryl ring; L₁ is absent; L₂ is selected from the group consisting of —C(R_{d}Rₑ)—, —(CH₂)ₙ—, —NH—, —O—, and —S—; n is 1, 2 or 3; Z is a member selected from the group consisting of hydroxy, hydroxyalkyl, R_{g}—O— and Rⱼ—NH—; R₁ is hydrogen, C₁₋₃ haloalkyl or C₁₋₃ alkyl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of heteroaryl and heteroarylalkyl, B is selected from the group consisting of an aryl ring and a heteroaryl ring; D is selected from the group consisting of an aryl ring and a heteroaryl ring; L₁ is selected from the group consisting of —NR_{c}—, —NR_{c}CH₂—, —O—, and —S—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of heteroaryl and heteroarylalkyl, B is selected from the group consisting of an aryl ring and a heteroaryl ring; D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; Rₑ is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a compound of formula (I) wherein A is selected from the group consisting of heteroaryl and heteroarylalkyl, B is selected from the group consisting of an aryl ring and a heteroaryl ring; D is selected from the group consisting of an aryl ring and a heteroaryl ring; L₁ is —C(RₐR_{b})—; L₂ is selected from the group consisting of —C(R_{d}Rₑ)—, —(CH₂)ₙ—, —NH—, —O—, and —S—; n is 1, 2 or 3; Z is a member selected from the group consisting of alkoxy, hydroxy, hydroxyalkyl, R_{g}—O— and Rⱼ—NH—; R₁ is hydrogen, C₁₋₃ haloalkyl or C₁₋₃ alkyl; Rₐ and R_{b} are each individually selected from the group consisting of hydrogen, alkyl, haloalkyl, hydroxy or Rₐ and R_{b} taken together with the atom to which they are attached form R_{f}—N═; R_{c} is selected from the group consisting of hydrogen, alkyl, aryl, haloalkyl and heteroaryl; R_{d} is selected from the group consisting of alkyl, haloalkyl, hydroxy and halo; Rₑ is selected from the group consisting of hydrogen, alkyl, haloalkyl, hydroxy and halo, or R_{d} and Rₑ taken together with the atom to which they are attached form oxo; R_{f} is selected from the group consisting of alkoxy, aryloxy, heteroaryloxy and hydroxy; R_{g} is H₂N—C(O)— or C₁₋₃ alkylHN—C—(O)—; and Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

Another embodiment of the present invention is directed toward a method of inhibiting ACC, comprising administrating a therapeutically effective amount of a compound of formula (I).

Another embodiment of the present invention is directed toward a method of inhibiting ACC-1, comprising administering a therapeutically effective amount of a compound of formula (I).

The method comprising a compound selected from the group consisting of

Another embodiment of the present invention is directed toward a method of inhibiting ACC-2, comprising administering a therapeutically effective amount of a compound of formula (I).

The method comprising a compound selected from the group consisting of

Another embodiment of the present invention is directed toward a method of treating metabolic syndrome, comprising administering a therapeutically effective amount of a compound of formula (I).

Another embodiment of the present invention is directed toward a method of treating type II diabetes, comprising administering a therapeutically effective amount of a compound of formula (I).

Another embodiment of the present invention is directed toward a method of treating obesity, comprising administering a therapeutically effective amount of a compound of formula (I).

Another embodiment of the present invention is directed toward a pharmaceutical composition comprising a therapeutically effective amount of a compound of formula (I) in combination with a pharmaceutically suitable carrier.

### DEFINITIONS

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. Alkenes of the present invention may be substituted with groups which are members selected from the group consisting of alkoxycarbonyl, alkylcarbonyl, aryl, cycloalkyl, cyano, halogen, heteroaryl, heteroarylalkyl, heteroarylcarbonyl, heterocycle, heterocyclealkyl, heterocyclecarbonyl,.hydroxy, and hydroxyalkyl.

Representative examples of alkenyl include, but are not limited to, ethenyl, 1-chloropropene, 2-propenyl, 2-methyl-2-propenyl, 2-phenylpropenyl, 2-butenylnitrile, pent-3-en-1-ol, 1-but-2-enyl-pyrrolidine, 3-butenyl, 4-pentenyl, 5-hexenyl, 2-heptenyl, 2-methyl-1-heptenyl, and 3-decenyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxy-NH—C(O)—,” as used herein, refers to an alkoxy group, as defined herein, appended to the parent molecular moiety through a —NH—C(O)— group.

The term “alkoxycarbonyl-NH-alkyl-NHC(O)—,” as used herein, refers to an alkoxycarbonyl group, as defined herein, appended to the parent molecular moiety through a NH-alkyl-NHC(O)—, as defined herein.

The term “alkyl-NH—C(O)—,” as used herein, refers to an alkyl group, as defined herein, appended to the parent molecular moiety through a —NH—C(O)— group.

The term —NH-alkyl-NH—C(O)—,” as used herein, refers to a —NH— group, as defined herein, appended to the parent molecular moiety through an alkyl-NH—C(O)— group, as defined herein.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “C₁₋₆ alkyl,” as used herein, refers to a straight or branched chain hydrocarbon containing from 1 to 6 carbon atoms. Representative examples of C₁₋₆ alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylpentyl and 3-methylpentyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, refers to a monocyclic-ring system, or a bicyclic- or a tricyclic-fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of the present invention can be substituted with 1, 2, or 3 substituents wherein each substituent occurrence is independently selected from alkyl, alkenyl, alkoxy, alkoxyalkyl, alkoxyalkylcarbonyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, benzyl, carboxy, carboxyalkyl, cyano, cyanoalkyl, cycloalkyl, formyl, halogen, haloalkyl, heterocycle, tetrazole, heterocyclealkyl, hydroxy, hydroxyalkyl, mercapto, nitro, phenyl, RᵣRₛN—, RₚR_{q}NC(O)—, and RₚR_{q}NS(O)₂—, wherein Rᵣ and Rₛ are each independently selected from the group consisting of alkyl, alkylcarbonyl, alkoxycarbonyl, alkylsulfonyl and haloalkyl, and Rₚ and R_{q} are each independently selected from the group consisting of hydrogen, alkyl and haloalkyl and wherein the heterocycle and the heterocycle of heterocyclealkyl may be optionally substituted with 1, 2 or 3 substitutents selected from the group consisting of alkyl, alkenyl, alkoxy, alkoxyalkyl, alkoxyalkylcarbonyl, alkoxycarbonyl, alkoxycarbonylalkyl, alkyl, alkylcarbonyl, alkylsulfonyl, alkylthio, alkynyl, carboxy, carboxyalkyl, cyano, cyanoalkyl, formyl, halogen, haloalkyl, hydroxy, and hydroxyalkyl.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl and naphthoyl.

The term “aryloxy,” as used herein, refers to an aryl group, as defined herein, appended to the parent molecular moiety through an oxygen atom. Representative examples of aryloxy include, but are not limited to, phenoxy, naphth-2-yloxy and naphth-1-yloxy.

The term “aryloxyalkyl,” as used herein, refers to an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl and 3-bromophenoxymethyl.

The term “carbonyl,” as used herein, refers to a —C(O)— group.

The term “carboxy,” as used herein, refers to a HO₂C— group.

The term “cyano,” as used herein, refers to a NC— group.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo(3.1.1)heptane, bicyclo(2.2.1)heptane, bicyclo(2.2.2)octane, bicyclo(3.2.2)nonane, bicyclo(3.3.1)nonane, and bicyclo(4.2.1)nonane. Alternatively, the bicyclic cycloalkyl ring systems are exemplified by a cycloalkyl ring appended to the parent molecular moiety which is fused to a distal ring, wherein the distal ring is selected from the group consisting of phenyl, heteroaryl and heterocycle. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo(3.3.1.0^{3,7})nonane and tricyclo(3.3.1.1^{3,7})decane (adamantane).

The cycloalkyl groups of this invention may be substituted with 0, 1, 2 or 3 substituents wherein each substitutent occurrence is selected from alkyl, alkylcarbonyl, alkoxy, alkoxycarbonyl, alkenyl, alkynyl, cyano, halogen, haloalkyl, hydroxy, hydroxyalkyl, nitro, RᵣRₛN—, RₚR_{q}NC(O)—, and RₚR_{q}NS(O)₂—, wherein Rᵣ and Rₛ are each independently selected from the group consisting of alkyl, alkylcarbonyl, alkoxycarbonyl, alkylsulfonyl and haloalkyl, and Rₚ and R_{q} are each independently selected from the group consisting of hydrogen and alkyl.

The term “cycloalkylalkyl,” as used herein, refers to a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “halo” or “halogen,” as used herein, refers to Cl—, Br—, I— or F—.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “halocycloalkyl,” as used herein, refers to at least one halogen, as defined herein, appended to the parent molecular moiety through an cycloalkyl group, as defined herein.

The term “heteroaryl,” as used herein, means a monocyclic or a bicyclic ring. The monocyclic heteroaryl rings of the present invention may exist as a 5 or 6 membered ring. The 5 membered heteroaryl ring has two double bonds and contains one, two, three or four heteroatoms independently selected from the group consisting of N, O, and S. The 6 membered heteroaryl ring has three double bonds and contains one, two, three or four heteroatoms independently selected from the group consisting of N, O, and S. The bicyclic heteroaryl ring consists of the 5 or 6 membered heteroaryl ring appended to the parent molecular moiety which is fused to a distal ring, wherein the distal ring is selected from the group consisting of aryl, cycloalkyl, and heteroaryl. Nitrogen heteroatoms contained within the heteroaryl may be optionally oxidized to the N-oxide or optionally protected with a nitrogen protecting group known to those of skill in the art. The heteroaryl is connected to the parent molecular moiety through any carbon atom or any nitrogen atom contained within the heteroaryl. Representative examples of heteroaryl include, but are not limited to, benzothienyl, benzoxadiazolyl, cinnolinyl, 5,6-dihydroisoquinolinyl, 7,8-dihydroisoquinolinyl, 5,6-dihydroquinolinyl, 7,8-dihydroquinolinyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, pyridinium N-oxide, quinolinyl, 5,6,7,8-tetrahydroisoquinolinyl, 5,6,7,8-tetrahydroquinolinyl, tetrazolyl, thiadiazolyl, thiazolyl, thienopyridinyl, thienyl, triazolyl, and triazinyl.

According to the present invention, heteroaryls of the present invention can be substituted with 1, 2, or 3 substituents independently selected from alkenyl, alkoxy, alkoxyalkyl, alkoxyalkynyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, halogen, haloalkyl, heterocycle, hydroxy, hydroxyalkyl, hydroxyalkynyl, hydroxycycloalkyl, mercapto, nitro, piperazinyl, pyridinyl, pyrazinyl, thiophen-yl, tetrahydropyridinyl, alkoxy-N═C(alkyl)alkyl-, HO—N═C(alkyl)-, RₛₛRₜₜN—, RₛₛRₜₜNcarbonyl, RₛₛRₜₜNalkyl, RₛₛRₜₜNalkylNHcarbonyl, RₛₛRₜₜNalkynyl wherein Rₛₛ and Rₜₜ are each independently selected from the group consisting of alkyl, alkylcarbonyl, alkoxycarbonyl, alkylsulfonyl and haloalkyl.

The term “heteroarylalkyl,” as used herein, refers to a heteroaryl, as defined herein, appended to the parent molecular moiety through an alkyl group, as defined herein.

The term “heteroarylcarbonyl,” as used herein, refers to a heteroaryl, as defined herein, appended to the parent molecular moiety through a carbonyl group, as defined herein.

The term “heteroaryloxy,” as used herein, refers to a heteroaryl, as defined herein, appended to the parent molecular moiety through an oxy group, as defined herein.

The term “heterocycle” or “heterocyclic” as used herein, means a monocyclic ring or a bicyclic ring or a tricyclic ring. The monocyclic ring consists of a 3, 4, 5, 6 or 7 membered ring which contains at least one heteroatom independently selected from the group consisting of oxygen, nitrogen and sulfur. The 3 or 4 membered ring contains 1 heteroatom. The 5 membered ring contains zero or one double bond and one, two or three heteroatoms. The 6 or 7 membered ring contains zero, one or two double bonds and one, two or three heteroatoms. Representative examples of the monocyclic heterocyclic ring include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocyclic ring consists of the monocyclic heterocyclic ring appended to the parent molecular moiety fused to a distal ring, wherein the distal ring is selected from the group consisting of aryl and cycloalkyl. Representative examples of the bicyclic heterocyclic ring include, but are not limited to, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. The tricyclic heterocyclic ring consists of the bicyclic heterocyclic ring fused to a phenyl group or the bicyclic heterocyclic ring fused to a cycloalkyl group or the bicyclic heterocyclic ring fused to a cycloalkenyl group or the bicyclic heterocyclic ring fused to another monocyclic heterocyclic ring. Representative examples of tricyclic heterocyclic ring include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

According to the present invention, heterocycles can be substituted with 1, 2 or 3 substituents wherein each substitutent occurrence is independently selected from alkenyl, alkoxy, alkoxyalkyl, alkoxyalkynyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, halogen, haloalkyl, hydroxy, hydroxyalkyl, hydroxyalkynyl, hydroxycycloalkyl, mercapto, nitro, piperazinyl, pyridinyl, pyrazinyl, thiophen-yl, tetrahydropyridinyl, alkoxy-N═C(alkyl)alkyl-, HO—N═C(alkyl)-, RₛₛRₜₜN—, RₛₛRₜₜNcarbonyl, RₛₛRₜₜNalkyl, RₛₛRₜₜNalkylNHcarbonyl, RₛₛRₜₜNalkynyl wherein Rₛₛ and Rₜₜ are each independently selected from the group consisting of alkyl, alkylcarbonyl, alkoxycarbonyl, alkylsulfonyl and haloalkyl, wherein the aryl, the aryl of arylalkoxy, the aryl of arylalkyl, the aryl of arylcarbonyl, and the aryl of aryloxy can be substituted with 1, 2 or 3 substitutents selected from the group consisting of alkyl, alkenyl, alkoxy, alkoxyalkyl, alkoxyalkylcarbonyl, alkoxycarbonyl, alkoxycarbonylalkyl, alkyl, alkylcarbonyl, alkylsulfonyl, alkylthio, alkynyl, carboxy, carboxyalkyl, cyano, cyanoalkyl, formyl, halogen, haloalkyl, hydroxy, and hydroxyalkyl.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, pyridin-3-ylcarbonyl and 2-pyrimidin-2-ylcarbonyl and the like.

The term “hydroxy,” as used herein, refers to an —OH group.

The term “hydroxyalkyl,” as used herein, refers to a hydroxy group, as defined herein, appended to the parent molecular moiety through an alkyl group, as defined herein. Representative examples of hydroxyalkyl include, but are not limited to, 2-hydroxyethyl, 2-hydroxypropyl, 3-hydroxybutyl and the like.

The term “nitro,” as used herein, refers to a —NO₂ group.

The term “oxo,” as used herein, referes to a ═O group.

The term “NH-alkyl-NHC(O)—,” as used herein, refers to an NH-alkyl group, as defined herein, appended to the parent molecular moiety through a NHC(O)— group.

The term “NH-alkyl-,” as used herein, refers to an NH atom, appended to the parent molecular moiety through an alkyl group.

The present invention is also directed to a method of inhibiting acetyl-CoA carboxylase (ACC). By inhibiting ACC, the compounds of the present invention may be useful as therapeutic agents for the treatment or prevention of disorders such as but not limited to metabolic syndrome, type B diabetes, obesity, atherosclerosis and cardiovascular disease. Therefore, according to an embodiment of the present invention compounds of formula (I), may be useful for the treatment of metabolic syndrome, type II diabetes, obesity, atherosclerosis and cardiovascular disease.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like. The present invention contemplates pharmaceutically suitable salts formed at the nitrogen of formula (I).

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I) for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described herein and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

Inhibition of ACC by the compounds of the present invention can be delayed by using a liquid suspension of crystalline or amorphous material with poor water solubility. The rate of absorption of the compounds depends upon their rate of dissolution which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient that is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of a compound of formula (I) to effectively ameliorate disorders by inhibiting ACC at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to inhibit the action of ACC in single or divided doses can be in amounts, for example, from about 0.1 to 50 mg/kg body weight. In a more preferred range, compounds of the present invention inhibit the action of ACC in a single or divided doses from about 1 to 25 mg/kg body weight. Single dose compositions can contain such amounts or submultiple doses thereof of the compounds of the present invention to make up the daily dose. In general, treatment regimens comprise administration to a patient in need of such treatment from about 1 mg to about 1000 mg of the compounds per day in single or multiple doses.

### Biological Data

The ACC2 enzymatic assay has been developed using either crude digitonin lysates of hACC2 overexpressing HEK 293 cells or recombinant human ACC2 expressed in baculovirus/Sf9 system. In both cases in order to increase the expression and solubility of the protein, a chimeric version of ACC2 (“mito-minus”), in which the N-terminal transmembrane domain (1-275 aa's of ACC2) was replaced with the corresponding ACC1 sequence (1-133 aa's). The enzymatic assay measures ACC mediated incorporation of [14C] CO2 into [¹⁴C]-Malonyl CoA. Mono-Avidin purified rat liver ACC1 was used as ACC1 enzyme source for the ACC-1 activity assay. The assay was preformed in 40 μL reaction in a 96-well plate format. The 1× assay buffer contains 50 mM Hepes/NaOH, pH 7.5, 10 mM citrate, 20 mM MgCl₂ and 0.075% BSA. First,20 μL of test compounds was dissolved in 1% DMSO in 1× assay buffer was dispensed into 96-well. Then, 10 μL of enzyme in 1× assay buffer was dispensed. The reaction was initiated by adding the following substrate mixture in 1× assay buffer: 2 mM ATP, 1 mM acetyl-CoA, and 17.6 mM NaHCO₃ (0.12 μCi). The reaction was carried out at room temperature for 40 minutes and the reaction was terminated by adding 50 μL of 1N HCl. The plate was air-dried in a fume hood at room temperature overnight. 20 μL of distilled water was added followed by adding 150 μL of SuperMix liquid scintillation fluid (PerkinElmer). The radioactivity was determined in PerkinElmer microbeta after vigorous shaking. The IC50 value was calculated from 8 dose response curve of test compounds.

TABLE 1
Inhibition of ACC1 and ACC2 Enzymatic Activities

| | ACC1 IC50 | ACC2 IC50 |
|---|---|---|
| Compound | (uM) | (uM) |
| A1 | 0.432 | 0.003 |
| A2 | 0.107 | 0.004 |
| A3 | 0.124 | 0.004 |
| A4 | 0.069 | 0.005 |
| A5 | 0.833 | 0.007 |
| B1 | 0.049 | 0.007 |
| B2 | 0.004 | 0.008 |
| B3 | 0.024 | 0.008 |
| B4 | 0.162 | 0.008 |
| B5 | 0.014 | 0.010 |
| C1 | 6.121 | 0.011 |
| C2 | 3.656 | 0.012 |
| C3 | 0.033 | 0.013 |
| C4 | 2.356 | 0.013 |
| C5 | 0.254 | 0.015 |
| D1 | 6.378 | 0.016 |
| D2 | 0.265 | 0.018 |
| D3 | 1.872 | 0.024 |
| D4 | 14.830 | 0.024 |
| D5 | 0.143 | 0.024 |
| E1 | 0.065 | 0.024 |
| E2 | 0.063 | 0.025 |
| E3 | 3.135 | 0.027 |
| E4 | 8.756 | 0.029 |
| E5 | 0.050 | 0.030 |
| F1 | 0.017 | 0.030 |
| F2 | 0.024 | 0.030 |
| F3 | 18.595 | 0.032 |
| F4 | 0.224 | 0.032 |
| F5 | 0.361 | 0.033 |
| G1 | 0.106 | 0.036 |
| G2 | 3.682 | 0.042 |
| G3 | 0.079 | 0.046 |
| G4 | 0.179 | 0.049 |
| G5 | 34.836 | 0.051 |
| H1 | 0.097 | 0.052 |
| H2 | 0.058 | 0.053 |
| H3 | 0.109 | 0.062 |
| H4 | 10.033 | 0.063 |
| H5 | 0.342 | 0.069 |
| J1 | 1.128 | 0.072 |
| J2 | 0.007 | 0.077 |
| J3 | 0.009 | 0.082 |
| J4 | 0.431 | 0.089 |
| J5 | 0.247 | 0.094 |
| K1 | 0.770 | 0.097 |
| K2 | 0.415 | 0.105 |
| K3 | 7.807 | 0.106 |
| K4 | 18.607 | 0.114 |
| K5 | 0.700 | 0.116 |
| L1 | 2.794 | 0.123 |
| L2 | 11.455 | 0.146 |
| L3 | 1.023 | 0.149 |
| L4 | 0.499 | 0.165 |
| L5 | 0.373 | 0.198 |
| M1 | 0.096 | 0.200 |
| M2 | 0.550 | 0.205 |
| M3 | 0.704 | 0.214 |
| M4 | 1.162 | 0.233 |
| M5 | 0.547 | 0.242 |
| N1 | 0.199 | 0.257 |
| N2 | 1.187 | 0.262 |
| N3 | 1.333 | 0.262 |
| N4 | 0.068 | 0.263 |
| N5 | 0.875 | 0.264 |
| P1 | 15.534 | 0.276 |
| P2 | 0.623 | 0.287 |
| P3 | 0.900 | 0.320 |
| P4 | 7.590 | 0.326 |
| P5 | 0.212 | 0.327 |
| Q1 | 0.565 | 0.350 |
| Q2 | 3.592 | 0.353 |
| Q3 | 4.532 | 0.358 |
| Q4 | 2.589 | 0.378 |
| Q5 | 30.000 | 0.381 |
| R1 | 30.000 | 0.404 |
| R2 | 0.882 | 0.410 |
| R3 | 0.169 | 0.418 |
| R4 | 0.701 | 0.421 |
| R5 | 0.640 | 0.422 |
| S1 | 15.592 | 0.423 |
| S2 | 7.873 | 0.433 |
| S3 | 5.194 | 0.441 |
| S4 | 30.000 | 0.449 |
| S5 | 0.442 | 0.465 |
| T1 | 1.864 | 0.495 |
| T2 | 11.920 | 0.498 |
| T3 | 5.900 | 0.500 |
| T4 | 5.767 | 0.511 |
| T5 | 0.086 | 0.522 |
| U1 | 8.271 | 0.534 |
| U2 | 7.301 | 0.583 |
| U3 | 23.768 | 0.587 |
| U4 | 0.056 | 0.627 |
| U5 | 4.238 | 0.633 |
| V1 | 3.773 | 0.638 |
| V2 | 12.844 | 0.680 |
| V3 | 2.216 | 0.709 |
| V4 | 100.000 | 0.769 |
| V5 | 5.753 | 0.786 |
| W1 | 0.174 | 0.826 |
| W2 | 0.078 | 0.859 |
| W3 | 30.000 | 0.874 |
| W4 | 2.086 | 0.905 |
| W5 | 0.303 | 0.940 |
| Y1 | 30.000 | 0.942 |
| Y2 | 3.619 | 0.950 |
| Y3 | 43.512 | 0.981 |
| Y4 | 12.310 | 0.985 |
| Y5 | 100.000 | 1.002 |
| Z1 | 29.962 | 1.012 |
| Z2 | 0.801 | 1.305 |
| Z3 | 2.108 | 1.347 |
| Z4 | 30.000 | 1.822 |
| Z5 | 0.543 | 2.042 |
| AA1 | 1.426 | 2.708 |
| AA2 | 0.609 | 3.214 |
| AA3 | 2.214 | 5.641 |
| AA4 | 2.794 | 5.982 |
| AA5 | 1.497 | 6.642 |
| BB1 | 0.646 | 6.968 |
| BB2 | 2.569 | 10.940 |
| BB3 | 0.466 | 30.000 |
| BB4 | 2.884 | 63.401 |
| BB5 | 7.292 | 100.000 |

Dysregulation of fatty acids metabolism contributes to decreased insulin sensitivity and the development of metabolic syndrome. ACC is known to modulate fatty acid synthesis and fatty acid oxidation in insulin responsive tissues such as liver, adipose and skeletal mucles. The ACC inhibitors of the present invention, have the potential to decrease de novo lipid synthesis and increase fat oxidation in vivo. Therefore, these chemotypes represent a novel method to treat insulin resistance/type 2 diabetes, as well as obesity, hypertension and hyperlipidemia.

In Vivo Data: Fatty Acid Oxidation via Respiratory Calorimetry

7-8 week old Sprague-Dawley rats were purchased from Charles River Laboratory (Wilmington, Mass.). The rats were single housed upon arrival and fed D-5001 pellets (Purina LabDiets, St. Louis, Mo.) consisting of 28% protein, 12% fat, and 60% CHO by calories. 5 days after their arrival, 16 rats were acclimated to the Oxymax indirect calorimetry system (Columbus Instruments, Columbus, Ohio) which measures O₂ consumption and CO₂ expiration. These values were used to calculate the respiratory exchange ratio (RER, VCO₂/VO₂). RER was used as an indicator to determine the type of metabolic fuel the subject animal was oxidizing (whole body) at the time of measurement: a ratio of 0.7 indicates the utilization of 100% fatty acids and a ratio of 1.0 indicates a utilization of 100% carbohydrates. Acclimation was accomplished by placing individual rats into the metabolic chambers from 2:00 PM on Day 1 until 8:00 AM on Day 2. The rats were then returned to their home cages. On Day 4 the rats were weighed at 10:00 AM and were individually placed into the Oxymax system with food and water at 10:30 AM. At least 90 minutes prior to the rats being placed into their chambers, the Oxymax system was turned on and allowed to warm-up. After the system was warmed up, the gas sensors were calibrated using a three part gas consisting of 20.50% O₂, 0.500% CO₂, and 79% N₂ (AGA Gas, Kenosha, Wis.). The flow rate to the chambers was 2.0 L/min. and the sample flow rate was 0.4 L/min. The food was removed from the cages at 11:00 AM. Immediately prior to their treatment with compound, the rats were separated into groups based on their RER. Each experimental group consisted of 5 to 8 rats (n=5-8). At 12:30 PM the rats were orally dosed with compound at 4 ml/kg. At approximately 1:30 PM the rats were subjected to meal challenge. The meal challenge consisted of 0.5 g/ml of raw cornstarch (6 g/kg: Jewel, Albertson's, Boise, Id.) in Glucerna (Abbott Laboratories, Abbott Park, Ill.). The first gas exchange data was collected at 2:30 PM or 2 hours post dose and continued until they were removed from the chamber at 8:00 AM on Day 5.

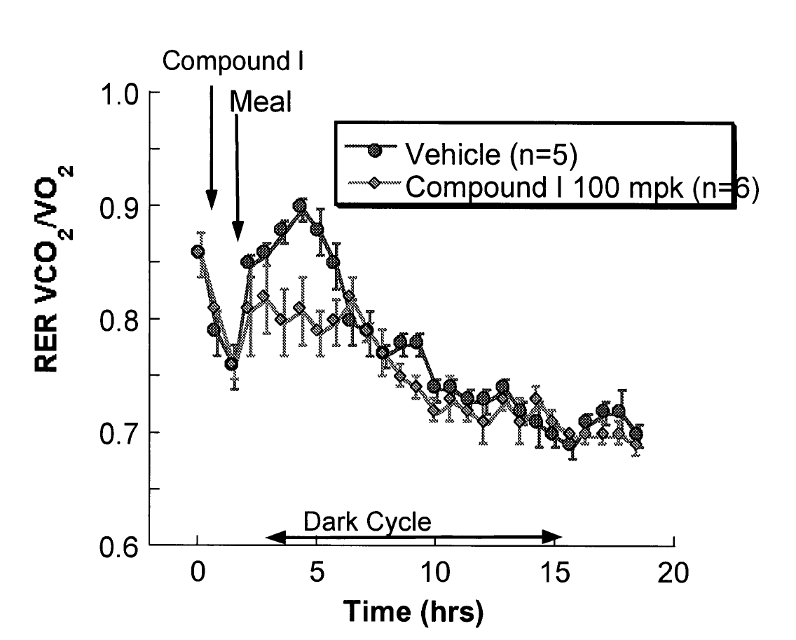

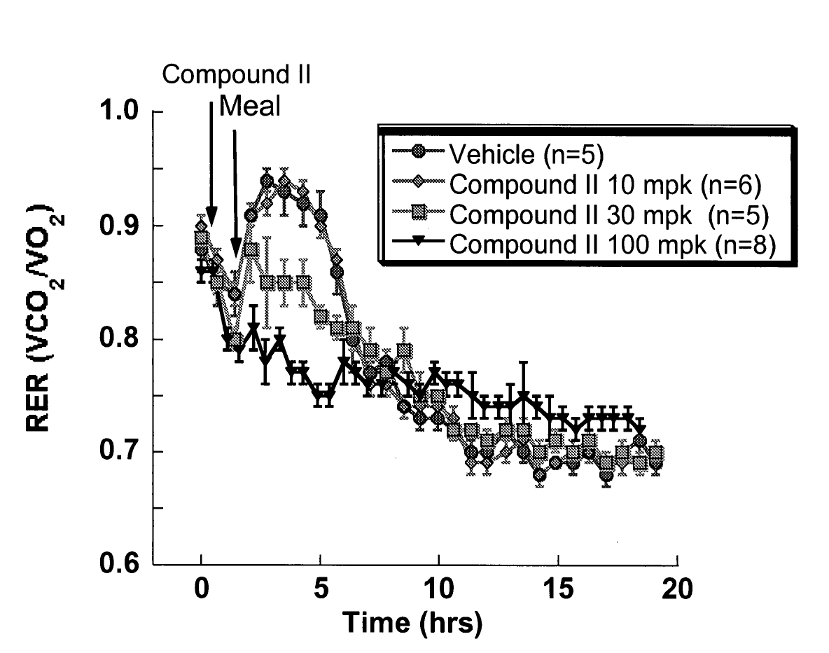

Compounds of the present invention were tested as ACC inhibitors in the Oxymax system, and the results shown in FIG. 1 and FIG. 2. The compounds of the present invention dose dependently decreased RER indicating an increase in whole body fat oxidation even in the presence of high carbohydrate meal. This data also indicates that the ACC inhibitors of the instant invention are bioactive.

Synthetic Methods

The compounds and processes of the present invention will be better understood in connection with the following synthetic Schemes and Experimentals which together illustrate the methods by which the compounds of the invention may be prepared. The synthesis of compounds of formula (I) wherein the groups A, B, D, L₁, L₂, Z, n, R₁ and Rₐ, R_{b}, R_{c}, R_{d}, Rₑ and R_{f} are as defined above unless otherwise noted below, are exemplified below.

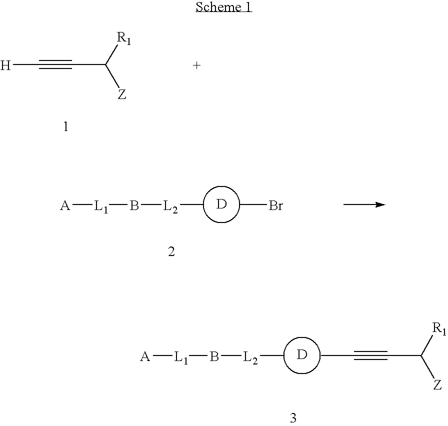

As shown in Scheme 1, compounds of general formula 3 which are representative of the compounds of the present invention, may be prepared by treating a compound of general formula 1 with a compound of general formula 2 in the presence of a palladium catalyst such as but not limited to dichlorobis(triphenylphosphine)-palladium (II), copper(I) iodide, a base such as but not limited to triethylamine in a solvent such as but not limited to tetrahydrofuran (THF) under heated conditions to provide a compound of general formula 3.

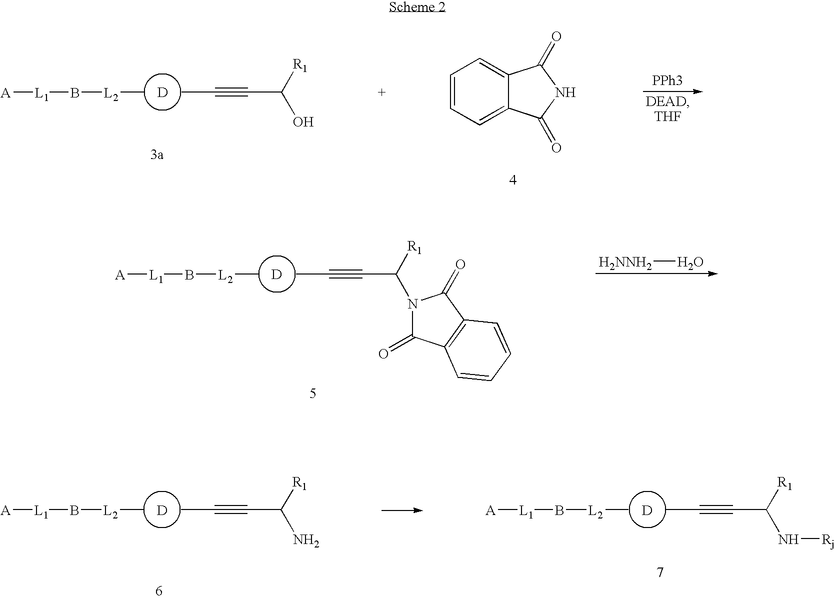

As shown in Scheme 2, compounds of general formula 3a, which may be prepared according to the procedure outlined in Scheme 1, when treated with phthalimide of formula 4 and triphenylphosphine, diethyl azodicarboxylate in a solvent such as THF will provide a compound of general formula 5. Compounds of general formula 5 when treated with hydrazine-mono hydrate in a solvent such as THF under heated conditions will provide the compound of general formula 6. The compound of general formula 6 when treated with compounds of formula Rⱼ—Y, wherein Rⱼ is defined within the scope of formula I and Y is a leaving group as known to one skilled in the art, and a base such as but not limited to triethylamine in solvents such as dichloromethane or THF will provide compounds of formula 7 which are representative of compounds of the present invention.

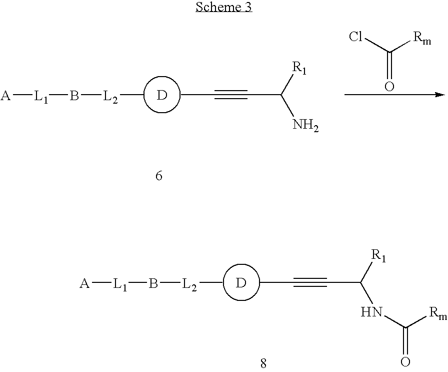

As outlined in Scheme 3, compounds of general formula 6 when treated with acid chlorides of formula Rₘ—C(O)—Cl (wherein Rₘ is alkyl and alkoxy) in the presence of a base such as tirethylamine or diisopropylethylamine in solvents such as but not limited to dichloromethane or THF will provide compounds of general formula 8 which are representative of compounds of the present invention.

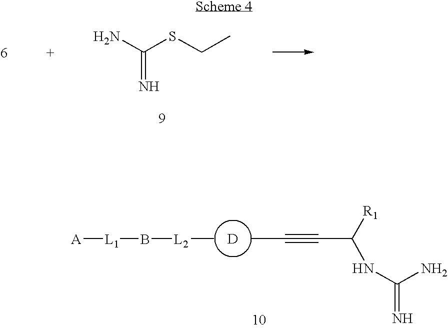

As outlined in Scheme 4, compounds of general formula 6 when treated with compounds of general formula 9 in THF under heated conditions will provide compounds of general formula 10 which are representative of compounds of the present invention.

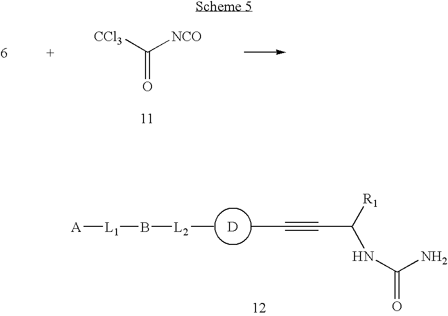

As outlined in Scheme 5, compounds of general formula 6 when treated with reagents of formula 11 will provide compounds of general formula 12 which are representative of compounds of the present invention.

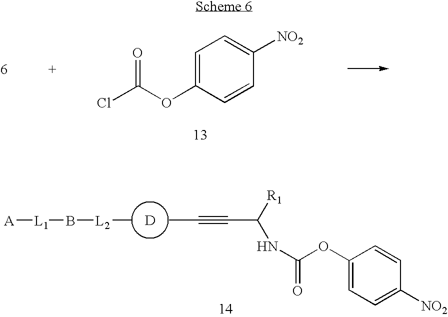

As outlined in Scheme 6, compounds of general formula 6 when treated with compounds of general formula 13 will provide compounds of general formula 14 which is used in Scheme 7 shown below.

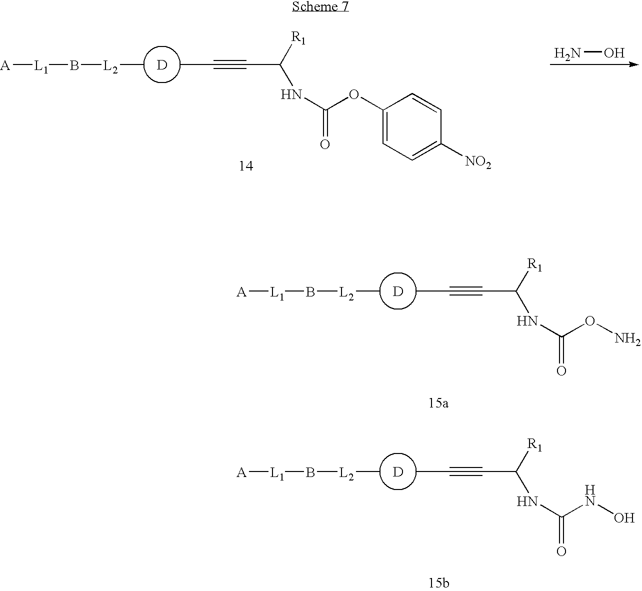

As outlined in Scheme 7, compound of general formula 14 when treated with hydroxylamine will provide both compounds of general formula 15a and 15b which are both representative of compounds of the present invention. Compounds of general formula 14 may also be used in the synthesis of other compounds representative of the present invention.

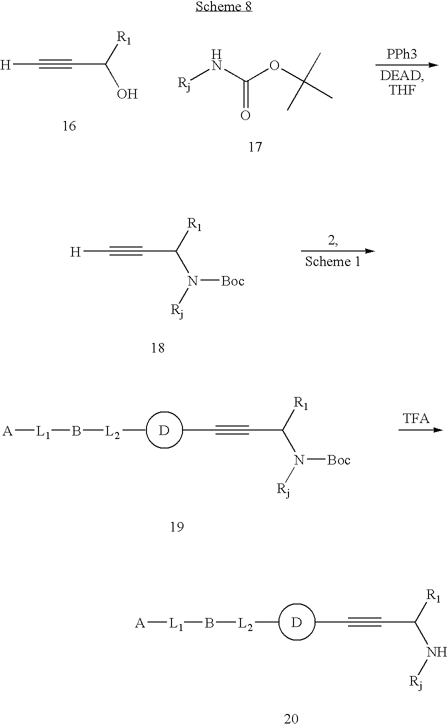

As outlined in Scheme 8, compounds of general formula 16 when treated with Boc protected amines of general formula 17 and triphenylphosphine and diethyl azodicarboxylate in THF will provide compounds of general formula 18. Compounds of general formula 18 when treated with a compound of general formula 2 according to the conditions outlined in Scheme 1, will provide acetylenes of general formula 19. Compounds of general formula 19 when treated with trifluoroacetic acid in dichloromethane will provide compounds of general formula 20 which are representative of compounds of the present invention.

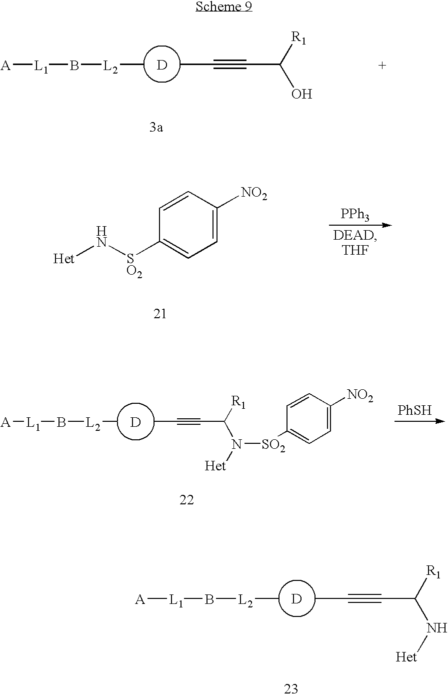

As outlined in Scheme 9, compounds of general formula 3a when treated with compounds of general formula 21, triphenylphosphine, diethyl azodicarboxylate in THF will provide compounds of general formula 22. Compounds of general formula 22 when treated with thiophenol will provide compounds of general formula 23 which are representative of compounds of the present invention.

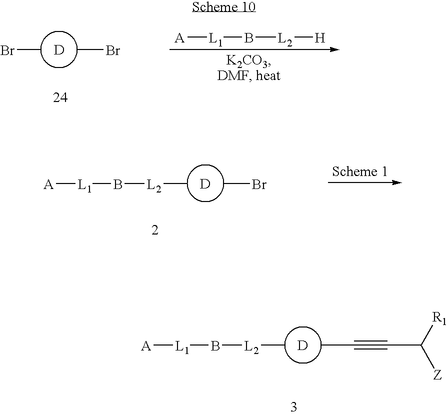

As outlined in Scheme 10, compounds of general formula 24 when treated with compounds of formula A-L₁-B-L₂-H, wherein L₂ is NH, O or S, along with potassium carbonate in DMF under heated conditions will provide compounds of general formula 2. Compounds of general formula 2 and compounds of general formula 1 when subjected to conditions outlined in Scheme 1 will provide compounds of general formula 3. This general strategy is outline further in the following schemes.

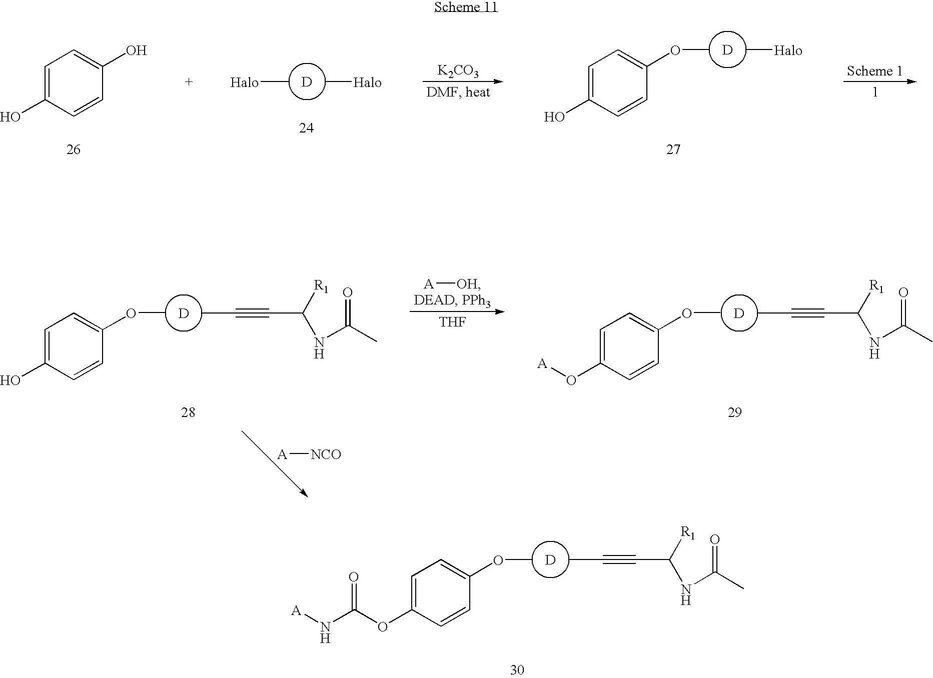

As outlined in Scheme 11, compounds of formula 26 when treated with compounds of general formula 24, potassium carbonate in DMF under heated conditions will provide compounds of general formula 27. Compounds of general formula 27 and compounds of general formula 1 when subjected to conditions outlined in Scheme 1 will provide compounds of general formula 28. Compounds of general formula 28 when treated with A-OH (wherein A is selected from the group consisting of alkenyl, alkoxyalkyl, alkyl, arylalkyl, cycloalkyl, cycloalkylalkyl, haloalkyl, heteroarylalkyl, and heterocyclealkyl), and triphenylphosphine and DEAD in THF will provide compounds of general formula 29 which are representative of compounds of the present invention when L₁ is —O—. Alternatively, compounds of general formula 28 when treated with isocyanates of formula A-NCO (wherein A of A-NCO is selected from the group consisiting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl) will provide compounds of general formula 30 which are representative of compounds of the present invention when L₁ is —NH—C(O)—O—.

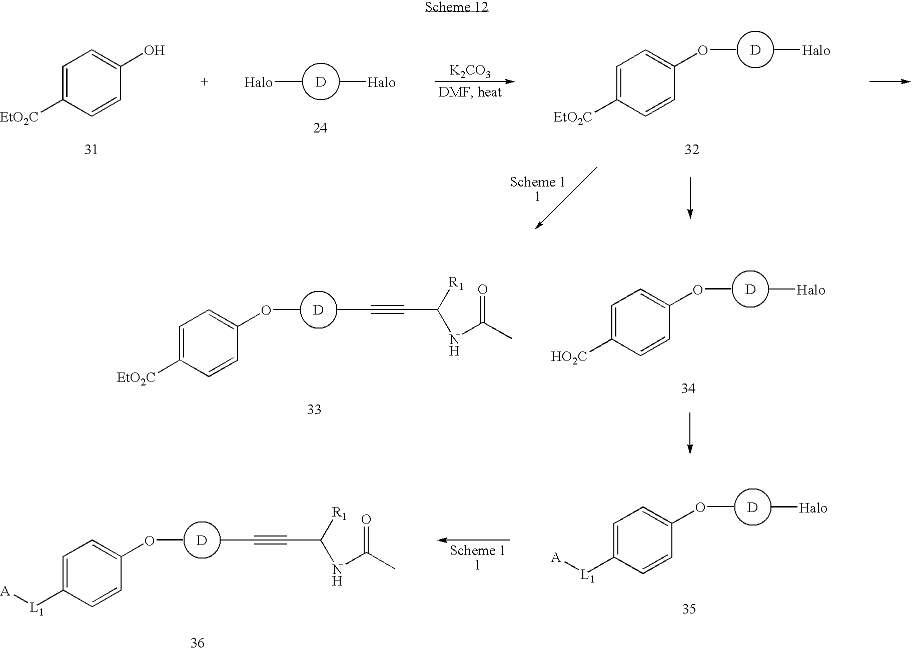

As outlined in Scheme 12, compounds of general formula 31 when treated with compounds of general formula 24, potassium carbonate in DMF under heated conditions will provide compounds of formula 32. Compounds of formula 32 and compounds of general formula 1 when subjected to conditions outlined in Scheme 1 will provide compounds of general formula 33 which are representative of compounds of the present invention when L₁ is —O—C(O)— and A is alkyl. Alternatively, compounds of formula 32 when treated with Lithium hydroxide or sodium hydroxide in solvents such as but not limited to aqueous isopropanol or aqueous methanol will provide compounds of general formula 34. Compounds of general formula 34 when treated with reagents known to activate carboxylic acids such as but not limited to N-ethyl-N-(3-dimethylaminopropyl)carbodiimide and an amine or oxalyl chloride in THF followed by the addition of an alcohol will provide compounds of general formula 35. Compounds of general formula 35 when subjected to conditions outlined in Scheme 1 will provide compounds of general formula 36 which are representative of compounds of the present invention.

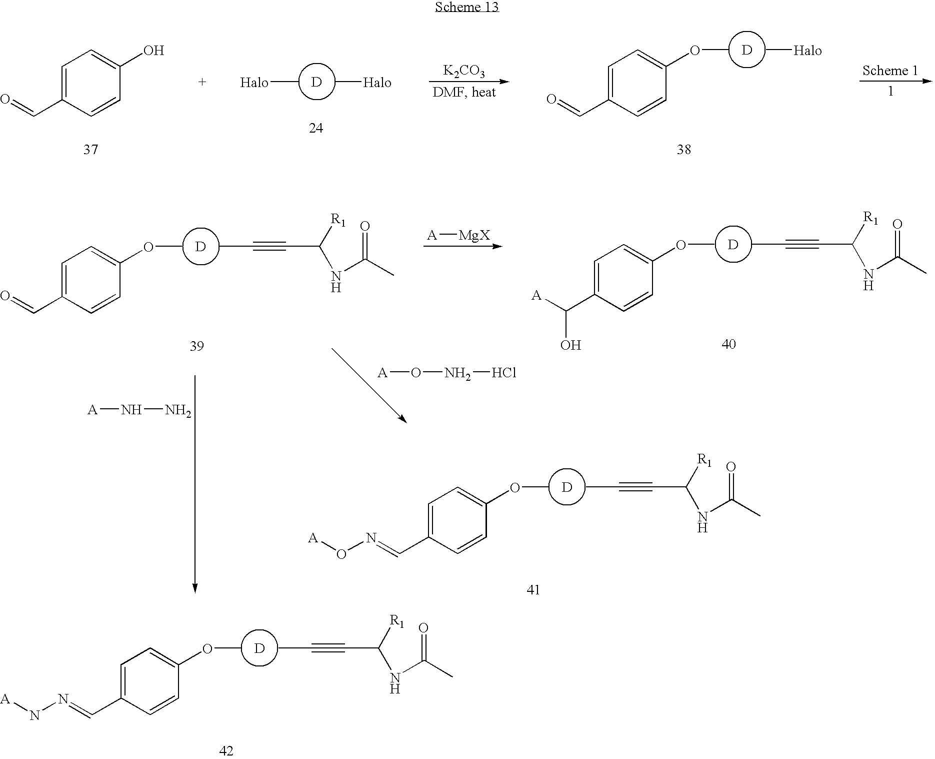

As outlined in Scheme 13, compounds of formula 37 when treated with compounds of formula 24, potassium carbonate in DMF under heated conditions will provide compounds of formula 38. Compounds of general formula 38 and compound of general formula 1 when subjected to the conditions outlined in Scheme 1 will provide compounds of general formula 39. Compound of general formula 39 when treated with reagents of general formula A-MgX, wherein X is chloro or bromo and A is as defined above, will provide compounds of general formula 40 which are representative of compounds of the present invention. Alternatively, compounds of general formula 39 when treated with A-O—NH₂ and a base such as pyridine in solvents such as THF will provide compounds of formula 41. Similarly, compounds of general formula 39 when treated with compounds of formula A-NH—NH₂ and a base such as pyridine will provide compounds of general formula 42 which are representative of the present invention.

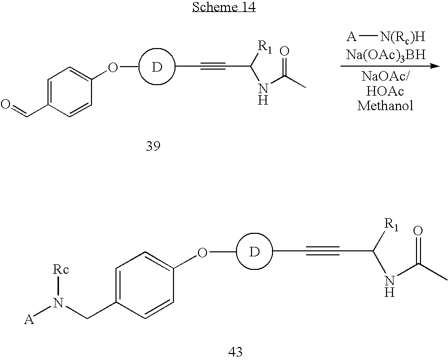

As outlined in Scheme 14, compounds of formula 39 may be treated with an amine of formula A-N(R_{c})—H in the presence of sodium tri(acetoxy) borohydride in methanol buffered with sodium acetate/acetic acid will provide compounds of general formula 43 which are representative of compounds of the present invention.

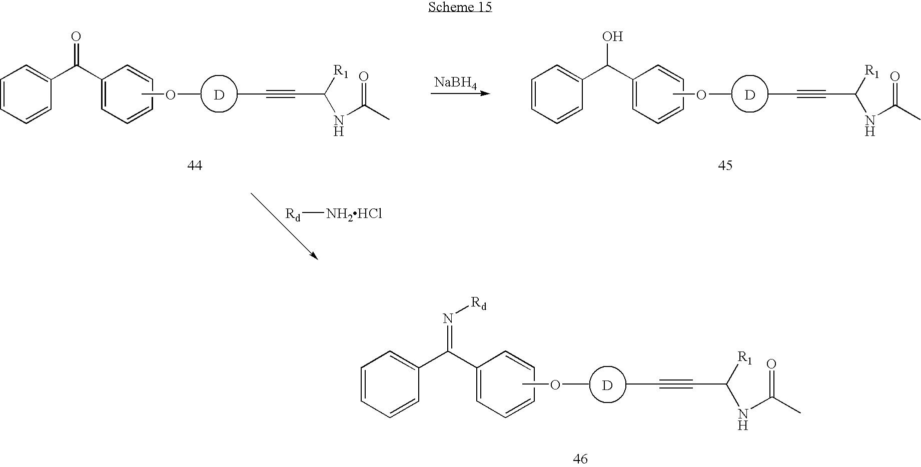

As outlined in Scheme 15, compounds of general formula 44, which may be obtained using the Schemes listed above, when treated with sodium borohydride in solvents such as methanol will provide compounds of general formula 45 which are representative of compounds of the present invention. Alternatively, compounds of general formula 44 when treated with compounds of formula R_{d}—NH₂:HCl along with pyridine in a solvent such as ethanol under heated conditions will provide compounds of general formula 46 which are representative of compounds of the present invention when L₁ is —C(RₐR_{b})— and wherein Rₐ and R_{b} taken together with the atom to which they are attached form R_{d}—N═.

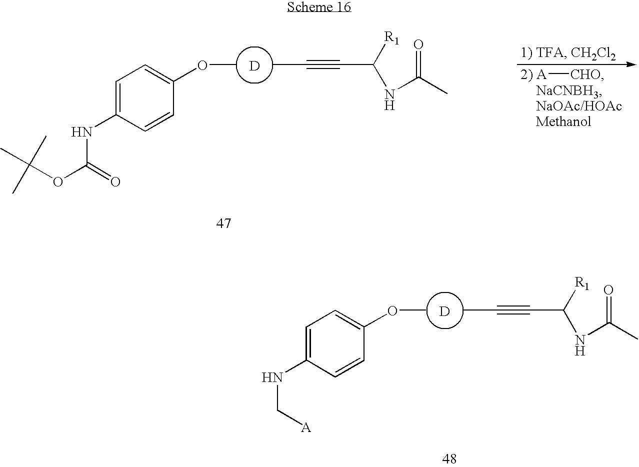

As outlined in Scheme 16, compounds of general formula 47, which may be obtained using the schemes listed above, when treated with trifluoroacetic acid in dichloromethane followed by treatment with A-CHO, sodium cyanoborohydride in a solvent such as but not limited to methanol buffered with sodium acetate and acetic acid will provide compounds of general formula 48 which are representative of compounds of the present invention.

Compounds of the invention were named by ACD/ChemSketch version 5.01 (developed by Advanced Chemistry Development, Inc., Toronto, ON, Canada) or were given names consistent with ACD nomenclature.

### Experimentals

### EXAMPLE 1

### N-(3-{2-[3-(cyclohexylmethoxy)phenoxy]-1,3-thiazol-5-yl}-1-methylprop-2-ynyl)acetamide

### EXAMPLE 1A

A solution of 2,5-dibromothiazole (5 g, 20.58 mmol) and resorcinol (4.53 g, 41.16 mmol) in DMF (100 mL) was treated with potassium carbonate (2.86g, 20.58 mmol) and the mixture was heated at 130° C. for 2 h. The reaction was cooled to 25° C., poured into water (400 mL) and extracted with diethyl ether (3×250 mL). The combined organic layers were washed with water (3×150 mL) and brine (150 mL), dried over sodium sulfate and concentrated on a rotary evaporator. The crude concentrate (6.4 g) was purified by flash chromatography on silica gel eluting with a solvent gradient from 10% to 25% ethyl acetate in hexanes to provide 3.6 g (64%) of Example 1a as awhite solid. ¹H NMR (300 MHz, DMSO-D6) δ ppm 6.67-6.88 (m, 2H) 7.28 (t, J=8.27 Hz, 1H) 7.51 (s, 1H) 9.90 (s, 1H); MS (ESI) m/z 273.6(M+H)⁺.

### EXAMPLE 1B

A solution of Example 1A (542 mg, 2 mmol), Example 5B (245 mg, 2.2 mmol), and Et₃N (858 μL, 6 mmol) in THF (15 mL) was degassed and treated with Pd(PPh₃)₂Cl₂ (70 mg, 0.1 mmol) and CuI (5 mg, 0.05 mmol). The reaction was heated under nitrogen at 75° C. for 2.5 h. The solvent was evaporated and the crude concentrate was purified by flash chromatography on silica gel eluting with a solvent gradient from 65% to 95% ethyl acetate in hexanes to provide 454 mg (75%) of Example 1B as a light yellow solid. ¹H NMR (300 MHz, DMSO-D6) δ ppm 1.33 (d, J=6.99 Hz, 3H) 1.81 (s, 3H) 4.69-4.92 (m, 1H) 6.65-6.85 (m, 2H) 7.26 (t, J=8.27 Hz, 1H) 7.50 (s, 1H) 8.41 (d, J=8.09 Hz, 1H) 9.91 (s, 1H); MS (ESI) m/z 302.8 (M+H)⁺.

### EXAMPLE 1C

To a solution of Example 1B (40 mg, 0.13 mmol), cyclohexylmethanol (25 uL, 0.19 mmol), and triphenylphosphine (50 mg, 0.19mmol) in THF (1 mL) was added diethyl azodicarboxylate (32 uL, 0.19 mmol) dropwise at ambient temperature under nitrogen. The reaction mixture was stirred for 4 hours and was concentrated in vacuo with a rotary evaporator. The crude concentrate was purified by reverse-phase HPLC on an Atlantis C18 column (1.9×10 cm, 5 μm particle size) using a gradient of 5% to 95% acetonitrile:0.1% aqueous TFA to provide 16 mg (31%) of Example 1C. ¹H NMR(300 MHz, METHANOL-D4) δ ppm 1.00-1.37 (m, 5H) 1.41 (d, J=6.80, 3H) 1.65-1.91 (m, 6H) 1.92(s, 3H) 3.78 (d, J=1.84 Hz, 2H) 4.87-4.94 (m, 1H) 6.67-6.98 (m, 3H) 7.22-7.45 (m, 2H) 8.51 (d, 1H); MS (ESI) m/z 399.2 (M+H)⁺.

### EXAMPLE 2

### N-{3-[4-(4-isopropoxyphenoxy)phenyl]-1-methylprop-2-ynyl}acetamide

### EXAMPLE 2A

### 4-isopropoxyphenol

To a mixture of hydroquinone (20.0 g, 0.182 mmol), 2-iodopropane (30.9 g, 0.182 mmol) in ethanol (25 mL) at refluxing was added KOH (88%, 12.2 mg, 0.191 mmol) in water (30 mL) over a period of 60 min. The resulting mixture was refluxed for 3 hours. The mixture was poured into 1N NaOH and extracted with ether (1×). The aqueous layer was acidified with 10% HCl to pH ˜5 and extracted with ether (2×). The combined extracts were washed with brine (1×), dried over MgSO₄ and concentrated. The residue was purified on silica gel eluting with ethyl acetate:hexane (1:8) to give the desired product as a brownish liquid (13.01 g, 47.0%).

### EXAMPLE 2B

### 4-(4-isopropoxyphenyl)phenyl bromide

To a mixture of Example 2A (505 mg, 3.31 mmol), 1,4-dibromobenzene (2.34 g, 9.92 mmol.), K₂CO₃ (960 mg, 6.87 mmol) and pyridine (20 mL) at 80° C. was added Cu(II) oxide (650 mg, 8.17 mmol). After the addition, the mixture was refluxed vigorously for 20 h. After cooling, methylene chloride was added and the mixture was filtered through Celite. The filtrate was concentrated to dryness. The residue was dissolved in ether, which was washed with 10% HCl (2×), 1N NaOH (2×), brine (1×), dried over MgSO₄, and concentrated to dryness. The residue was purified on on silica gel eluting with hexane and ethyl acetate gradient to give the desired product as a white solid (477 mg, 47%).

### EXAMPLE 2C

### N-{3-[4-(4-isopropoxyphenoxy)phenyl]-1-methylprop-2-ynyl}acetamide

A mixture of Example 2B (129 mg, 0.421 mmol), N-(1-methyl-prop-2-ynyl)-acetamide (60 mg, 0.540 mmol), CuI (4 mg, 0.021 mmol), Pd(PPh₃)₂Cl₂ (15 mg, 0.0214 mmol) and triethylamine (0.45 mL, 3.22 mmol) in acetonitrile (1.5 mL) was heated to 100° C. for 20 min under microwave. The mixture was concentrated and the residue was purified on silica gel eluting with hexane and ethyl acetate gradient to give the desired product as a white solid (66.2 mg). MS(DCI): m/z 338 (M+H); ¹H NMR (300 MHz, CHLOROFORM-D) δ ppm 1.34 (d, J=5.88 Hz, 6H), 1.48 (d, J=6.62 Hz, 3H), 2.00 (s, 3H), 4.42-4.56 (m, 1H), 4.97-5.08 (m, 1H), 6.82-6.91 (m, 4H), 6.92-6.99 (m, 2H), 7.30-7.37 (m, 2H).

### EXAMPLE 3

### N-(3-{6-[3-(cyclohexylmethoxy)phenoxy]pyridin-3-yl}-1-methylprop-2-ynyl)acetamide

### EXAMPLE 3A

A solution of 5-bromo-2-fluoropyridine (5 g, 28.41 mmol), and resorcinol (6.3 g, 56.82 mmol) in DMF (200 mL) was treated with potassium carbonate (4 g, 28.41 mmol) and the mixture was heated at 130° C. for 2 hours. The reaction was cooled to 25° C., poured into water (500 mL) and extracted with diethyl ether (3×200 mL). The combined organic layers were washed with water (3×200 mL) and brine (200 mL), dried over sodium sulfate and concentrated on a rotary evaporator. The crude concentrate (8.4 g) was purified by flash chromatography on silica gel eluting with a solvent gradient from 10% to 25% ethyl acetate in hexanes to provide 3.06 g (40%) of Example 3A as a white solid. 1H NMR (300 MHz, DMSO-D6) δ ppm 6.39-6.58 (m, 2H) 6.63 (dd, J=8.46, 2.21 Hz, 1H) 6.99 (d, J=8.82 Hz, 1H) 7.19 (t, J=8.09 Hz, 1H) 8.04 (dd, J=8.64, 2.76 Hz, 1H) 8.29 (d, J=2.57 Hz, 1H) 9.65 (s, 1H); MS (ESI) m/z 267.8 (M+H)⁺.

### EXAMPLE 3B

A solution of Example 3A (1.37 g, 5.15 mmol), Example 5B (687 mg, 6.18 mmol) and Et-₃N (2.15 mL, 15.44 mmol) in THF (50 mL) was degassed and treated with Pd(PPh₃)₂Cl₂ (181 mg, 0.26 mmol) and CuI (25 mg, 0.13 mmol). The reaction was heated under nitrogen at 80° C. for 3 h. The solvent was evaporated and the crude concentrate was purified by flash chromatography on silica gel eluting with a solvent gradient from 65% to 95% ethylacetate in hexanes to provide 940 mg (62%) of Example 3B as a light yellow solid. ¹H NMR (300 MHz, DMSO-D6) δ ppm 1.36 (d, J=6.99 Hz, 3H) 1.83 (s, 3H) 4.70-5.01 (m, 1H) 6.43-6.58 (m, 2H) 6.63 (dd, J=8.09, 2.21 Hz, 1H) 6.97 (d, J=8.46 Hz, 1H) 7.19 (t, J=8.09 Hz, 1H) 7.85 (dd, J=8.46, 2.21 Hz, 1H) 8.21 (d, J=2.21 Hz, 1H) 8.43 (d, J=7.72 Hz, 1H) 9.65 (s, 1H); MS (ESI) m/z 297.0 (M+H)⁺.

### EXAMPLE 3C

To a solution of Example 3B (50 mg, 0.17 mmol), cyclohexylmethanol (32 uL, 0.25 mmol), and triphenylphosphine (66 mg, 0.25 mmol) in THF (1.5 mL) was added diethyl azodicarboxylate (40 uL, 0.25 mmol) dropwise at ambient temperature over 2 min under nitrogen. The reaction mixture was stirred for 4 hours and was concentrated in vacuo with a rotary evaporator. The crude concentrate was purified by reverse-phase HPLC on an Atlantis C18 column (1.9×10 cm, 5 μm particle size) using a gradient of 5% to 95% acetonitrile:0.1 % aqueous TFA to provide 26 mg (39%) of Example 3C. 1H NMR (300 MHz, DMSO-D6) δ ppm 0.93-1.12 (m, 2H) 1.11-1.32 (m, 3H) 1.36 (d, J=6.99 Hz, 3H) 1.58-1.81 (m, 6H) 1.83 (s, 3H) 3.76 (d, J=6.25 Hz, 2H) 4.75-4.92 (m, 1H) 6.63-6.73 (m, 2H) 6.75-6.85 (m, 1H) 7.00 (d, J=8.46 Hz, 1H) 7.30 (t, J=7.91 Hz, 1H) 7.86 (dd, J=8.46, 2.21 Hz, 1H) 8.21 (d, J=2.57 Hz, 1H) 8.43 (d, J=8.09 Hz, 1H); MS (ESI) m/z 393.1 (M+H)⁺.

### EXAMPLE 4

### N-{1-methyl-3-[4-(4-phenoxyphenoxy)phenyl]prop-2-ynyl}acetamide

### EXAMPLE 4A

### 4-(4-phenozyphenyl)phenyl bromide

The desired product was prepared by substituting 4-phenoxyphenol for 4-isopropoxyphenol in Example 2B.

### EXAMPLE 4B

### N-{1-methyl-3-[4-(4-phenoxyphenoxy)phenyl]prop-2-ynyl}acetamide

The desired product was prepared by substituting Example 4A for Example 2B in Example 2C. MS(DCI): m/z 372 (M+H); ¹H NMR (300 MHz, CHLOROFORM-D) δ ppm 1.48 (d, J=6.99 Hz, 3H), 2.01 (s, 3H), 4.96-5.10 (m, 1H), 5.70 (br, 1H), 6.86-6.95 (m, 2H), 6.97-7.06(m, 6H), 7.06-7.14 (m, 1H), 7.29-7.41 (m, 4H).

### EXAMPLE 5

### N-{3-[4-(4-benzylphenoxy)phenyl]-1-methylprop-2-ynyl}acetamide

### EXAMPLE 5A

### 1-benzyl-4-(4-bromophenoxy)benzene

Commercially available 4-benzyl phenol (1.0 g, 5.4 mmol) was combined with commercially available 1,4-dibromo-benzene (3.82 g, 16.0 mmol), K₂CO₃ (1.6 g, 11.6 mmol), CuO (1.0 g, 12.6 mmol) and pyridine (36 ml). The black reaction suspension was refluxed for 24 h, cooled to rt and filtered through a plug of celite. After a second filtration through celite the residue was concentrated by rotary evaporation and purified by flash column chromatography to provide Example 5A as a light yellow oil. MS (ESI) m/z 339.0 (M+H⁺); ¹H NMR (300 MHz, DMSO-d₆) δ ppm 7.52(d, J=12.0 Hz, 2H), 7.27 (m, 7H), 6.96 (d, J=9.0 Hz, 2H), 6.92 (d, J=9.0 Hz, 2H), 3.93 (s, 2H).

### EXAMPLE 5B

### N-(1-methylprop-2-ynyl)acetamide

Acetic anhydride (30 ml) was added dropwise to H₂SO₄ (50 ml) at 0° C. After the exotherm subsided and the temperature returned to 0° C., a solution of But-3-yn-2-ol (14.5 g, 0.21 mol) in CH₃CN (27 ml) was added dropwise at 0° C. After the addition was complete the reaction was placed in the refrigerator (10.0° C.) for 48 hours. At that time, the reaction was stirred at room temperature and treated with NaOH (5M, aq) at 0° C. until the solution was basic as indicated by pH paper. The basic solution was extracted multiple times with diethyl ether (at least 5 repetitions), and the combined organics were dried (Na₂SO₄), filtered and concentrated by rotary evaporation to provide a yellow solid. The solid was collected by filtration and sublimed (0.05 Torr, 100° C.) to provide pure Example 5B. ¹H NMR (300 MHz, DMSO-d₆) δ ppm 1.26 (d, J=6.62 Hz, 3H) 1.79 (s, 3H) 3.14 (d, J=1.84 Hz, 1H) 4.42-4.69 (m, 1H) 8.29 (d, J=7.72 Hz, 1H); ¹³C NMR (75 MHz, DMSO-d₆) δ ppm 168.5, 85.6, 72.1, 35.6, 22.4, 21.8. The spectral data were consistent with the published data (Gardner, J. N., et al, Can. J. Chem, 51, 1973, 1416-1418).

### EXAMPLE 5

### N-{3-[4-(4-benzylphenoxy)phenyl]-1-methylprop-2-ynyl}acetamide

Example 5A (138 mg, 0.41 mmol), Example 5B (60 mg, 0.54 mmol), CuI (3.0 mg, 0.015 mmol) and Et₃N (173 μl) were combined in CH₃CN (2.5 ml) and degassed with N₂ for 2 min. PdCl₂(PPh₃)₂ (14 mg, 0.02 mmol) was added and the reaction was capped and placed in the microwave for 40 min at 120° C. The reaction suspension was diluted with EtOAc and poured into NH₄Cl (aq, sat.). The layers were separated, the aqueous layer was extracted with EtOAc and the combined organics were dried (Na₂SO₄), filtered and concentrated by rotary evaporation. The residue was purified by flash column chromatography to provide Example 5. MS (ESI) m/z 370.8 (M+H⁺); ¹H NMR (300 MHz, DMSO-d₆) δ ppm 1.34 (d, J=6.99 Hz, 3H) 1.82 (s, 3H) 3.94 (s, 1H) 4.74-4.87 (m, 1H) 6.88-6.94 (d, J=9.0 Hz, 2H) 6.95-7.02 (d, J=9.0 Hz, 4H) 7.16-7.33 (m, 5H) 7.34-7.42 (m, 2H) 8.39 (d, J=8.09 Hz, 1H).

### EXAMPLE 6

### N-(3-{2-[3-(cyclohexyloxy)phenoxy]-1,3-thiazol-5-yl}-1-methylprop-2-ynyl)acetamide

Example 6 was prepared according to the procedures described in Example 1 substituting cyclohexanol for cyclohexylmethanol in Example 1c. The crude product was purified by reverse-phase HPLC on an Atlantis C18 column (1.9×10 cm, 5 μm particle size) using a gradient of 5% to 95% acetonitrile:0.1 % aqueous TFA to provide 15 mg (30 %) of Example 6 as an amorphous white solid. ¹H NMR (300 MHz, methanol) δ ppm 1.42 (d, J=6.99 Hz, 3H) 1.44-1.64 (m, 6H) 1.72-1.86 (m, 2H) 1.93 (s, 3H) 1.94-2.04 (m, 2H) 4.24-4.43 (m, 1H) 4.81-4.98 (m, 1H) 6.74-6.95 (m, 3H) 7.27-7.40 (m, 2H) 8.50 (d, J=7.35 Hz, 1H); MS (ESI) m/z 385.1(M+H)⁺.

### EXAMPLE 7

### N-{1-methyl-3-[6-(4-pentylphenoxy)pyridin-3-yl]prop-2-ynyl}acetamide

### EXAMPLE 7A

### 5-Bromo-2-(4-pentyl-phenoxy)-pyridine

4-Pentyl-phenol (279 mg, 1.7 mmol), 5-Bromo-2-fluoro-pyridine (300 mg, 1.7 mmol) and K₂CO₃ (258 mg, 1.87 mmol) were combined in DMSO (3.5 ml). The reaction was placed in the microwave for 30 min at 150° C. Upon completion, the reaction was poured into water and diluted with EtOAc. The layers were separated and the organics were washed with 15% NaOH and then with water again. The organics were combined, dried (Na₂SO₄), filtered and concentrated by rotary evaporation to provide crude Example 7A, which was used without further purification. MS (ESI) m/z 322.6 (M+H⁺); ¹H NMR (300 MHz, DMSO-d₆) δ ppm 0.83-0.91 (m, 3H) 1.23-1.36 (m, 4H) 1.51-1.64 (m, 2H) 2.55-2.60 (m, 2H) 7.03 (d, J=9.19 Hz, 2H) 7.22 (d, J=8.46 Hz, 2H) 8.02 (d, J=2.57 Hz, 1H) 8.05 (d, J=2.57 Hz, 1H) 8.26 (d, J=2.57 Hz, 1H).

### EXAMPLE 7

### N-{1-methyl-3-[6-(4-pentylphenoxy)pyridin-3-yl]prop-2-ynyl}acetamide

Example 7A (460 mg, 1.4 mmol), Example 5B (186 mg, 1.7 mmol), CuI (11.0 mg, 0.06 mmol) and Et₃N (592 μl) were combined in THF (3.0 ml) and degassed with N₂ for 2 min. PdCl₂(PPh₃)₂ (49 mg, 0.07 mmol) was added and the reaction was capped in a pressure tude (or vial) and heated at 80° C. for 1.5 h. The reaction suspension was cooled, diluted with EtOAc and poured into NH₄Cl (aq, sat.). The layers were separated, the aqueous layer was extracted with EtOAc and the combined organics were dried (Na₂SO₄), filtered and concentrated by rotary evaporation. The residue was purified by flash column chromatography to provide Example 7. MS (ESI) m/z 351.7 (M+H)⁺; ¹H NMR (300 MHz, DMSO-d₆) δ ppm 0.82-0.93 (m, 3H) 1.25-1.33 (m, 2H) 1.36 (d, J=6.99 Hz, 4H) 1.52-1.64 (m, 2H) 1.83 (s, 3H) 2.53-2.62 (m, 2H) 4.77-4.90 (m, 1H) 6.99 (d, J=8.46 Hz, 1H) 7.04 (d, J=8.46 Hz, 2H) 7.23 (d, J=8.46 Hz, 2H) 7.83 (d, J=2.57 Hz, 1H) 7.86 (d, J=2.21 Hz, 1H) 8.18 (d, J=2.21 Hz, 1H) 8.43 (d, J=8.09 Hz, 1H).

### EXAMPLE 8

### N-{1-methyl-3-[6-(4-phenoxyphenoxy)pyridin-3-yl]prop-2-ynyl}urea

### EXAMPLE 8A

### 5-Bromo-2-(4-phenoxy-phenoxy)-pyridine

A mixture of 4-phenoxyphenol (1.060 g, 5.684 mmol), 5-bromo-2-fluoropyridine (1.000 g, 5.682 mmol), K₂CO₃ (0.945 g, 6.837 mmol) in DMSO (10 mL) was heated to 160° C. under microwave for 30 min. The reaction mixture was poured into water, extracted with ether (1×). The ether layer was washed with 10% NaOH (1×), water (1×), brine (1×), dried over MgSO₄ and concentrated to give the desired product as a white solid (1.86 g, 95.6%).

### EXAMPLE 8B

### 2-{3-[6-(4-phenoxy-phenoxy)-pyridin-3-yl]-1-methyl-prop-2-ynyl}-isoindole-1,3-dione

A mixture of Example 8A (576 mg, 1.684 mmol), 2-(1-methyl-prop-2-ynyl)-isoindole-1,3-dione (370 mg, 1.857 mmol), CuI (16 mg, 0.084 mmol), Pd(PPh₃)₂Cl₂ (60 mg, 0.0855 mmol), triethylamine (2.0 mL, 14.3 mmol) in acetonitrile (8 mL) was heated under microwave at 100° C. for 30 min. The mixture was concentrated dryness by blowing nitrogen. The residue was dissolved in ethyl acetate, which was washed with water (1×), brine, dried over MgSO₄ and concentrated. The residue was purified on silica gel eluting with ethyl acetate and hexane to give the desired product as a yellowish solid (424.4 mg, 54.7%).

### EXAMPLE 8C

### N-{1-methyl-3-[6-(4-phenoxyphenoxy)pyridin-3-yl]prop-2-ynyl}urea

A mixture of Example 8B (424.4 mg, 0.9216 mmol) and hydrazine monohydrate (250 μL, 5.15 mml) in ethanol (10 mL) was heated to reflux for 1 h. After cooling to room temperature, the reaction mixture was filtered and the filtrate concentrated. The residue was dissolved in methylene chloride and filtered again to give the desired amine as a colorless liquid (320.7 mg), which was dissolved in in methylene chloride (8 mL) and cooled to 0° C. To this was added trichloroacetyl isocyanate (150 μL, 1.26 mmol) dropwise. The mixture was stirred at room temperature for 10 minutes. The solvent was removed under vacuum to give a yellow solid. This was dissolved in methanol (20 mL). A few mg of Na₂CO₃ and a few drops of water were added. The mixture was refluxed for 2 hours. The reaction was concentrated and the residue dissolved in ethyl acetate, which was washed with water (1×), brine (1×), dried over MgSO₄ and concentrated. The residue was purified on silica gel eluting with ethyl acetate and hexane to give the desired product as a white solid (226.7 mg, 65.9%). MS (DCI): m/z 374 (M+H); ¹H NMR (300 MHz, CHLOROFORM-D) δ ppm 1.51 (d, J=6.62 Hz, 3H), 4.37 (br, 2H), 4.60-4.71 (m, 1H), 4.75-4.89 (m, 1H), 6.84 (d, J=8.82 Hz, 1H), 6.99-7.15 (m, 8H), 7.31-7.39 (m, 2H), 7.68 (dd, J=8.46, 2.21 Hz, 1H)

### EXAMPLE 9

### N-{3-[6-(4-cyclopentylphenoxy)pyridin-3-yl]-1-methylprop-2-ynyl}acetamide

### EXAMPLE 9A

### 5-Bromo-2-(4-cyclopentyl-phenoxy)-pyridine

Example 9A was prepared according to the procedure outlined in Example 7A, substituting 4-Cyclopentyl-phenol for 4-Pentyl-phenol. MS (ESI) m/z 320.5 (M+H⁺); ¹H NMR (300 MHz, DMSO-d₆) δ ppm 1.43-1.84 (m, 6H) 1.95-2.09 (m, 2H) 2.90-3.06 (m, 1H) 7.03-7.06 (m, 2H) 7.28 (d, J=8.46 Hz, 2H) 8.02 (d, J=2.57 Hz, 1H) 8.05 (d, J=2.57 Hz, 1H) 8.26 (d, J=2.57 Hz, 1H).

### EXAMPLE 9

### N-{3-[6-(4-cyclopentylphenoxy)pyridin-3-yl]-1-methylprop-2-ynyl}acetamide

Example 9 was prepared according to the procedure outlined in Example 7, substituting Example 9A for Example 7A. MS (ESI) m/z 349.7 (M+H⁺); ¹H NMR (300 MHz, DMSO-d₆) δ ppm 1.36 (d, J=6.0 Hz, 3H) 1.44-1.80 (m, 5H) 1.83 (s, 3H) 1.96-2.09 (m, 2H) 2.89-3.06 (m, 1H) 4.76-4.90 (m, 1H) 6.99 (d, J=8.46 Hz, 1H) 7.06 (d, J=8.46 Hz, 2H) 7.29 (d, J=8.46 Hz, 2H) 7.83 (d, J=2.57 Hz, 1H) 7.86 (d, J=2.21 Hz, 1H) 8.18 (d, J=2.21 Hz, 1H) 8.43 (d, J=7.72 Hz, 1H).

### EXAMPLE 10

### N-{3-[6-(4-benzylphenoxy)pyridin-3-yl]-1-methylprop-2-ynyl}acetamide

### EXAMPLE 10A

### 2-(4-Benzyl-phenoxy)-5-bromo-pyridine

Example 10A was prepared according to the procedure outlined in Example 7A substituting 4-Benzyl-phenol for 4-Pentyl-phenol. MS (ESI) m/z 340.5 (M+H⁺); ¹H NMR (300 MHz, DMSO-d₆) δ ppm 3.95 (s, 2H) 7.01 (d, J=9.19 Hz, 2H) 7.05 (d, J=9.19 Hz, 2H) 7.24-7.34 (m, 5H) 8.01 (d, J=2.94 Hz, 1H) 8.04 (d, J=2.57 Hz, 1H) 8.24 (d, J=2.57 Hz, 1H).

### EXAMPLE 10

### N-{3-[6-(4-benzylphenoxy)pyridin-3-yl]-1-methylprop-2-ynyl}acetamide

Example 10 was prepared according to the procedure outlined in Example 7A, substituting Example 10A for Example 7A. MS (ESI) m/z 371.7 (M+H⁺); ¹H NMR (300 MHz, DMSO-d₆) δ ppm 1.36 (d, J=6.99 Hz, 3H) 1.83 (s, 3H) 3.65 (s, 1H) 3.95 (s, 2H) 4.77-4.90 (m, 1H) 6.95-7.09 (m, 3H) 7.24-7.35 (m, 5H) 7.83 (d, J=2.21 Hz, 1H) 7.86 (d, J=2.57 Hz, 1H) 8.17 (d, J=1.84 Hz, 1H) 8.43 (d, J=8.09 Hz, 1H).

### EXAMPLE 11

### N-(3-{2-[4-(cyclopentylmethoxy)phenoxy]-1,3-thiazol-5-yl}-1-methylprop-2-yl)acetamide

### EXAMPLE 11A

### 4-[(5-bromo-1,3-thiazol-2-yl)oxy]phenol

A mixture of hydroquinone (440 mg, 4.0 mmol), 2,5-dibromothiazole (486 mg, 2.0 mmol) and potassium carbonate (276 mg, 2.0 mmol) in DMF (4 mL) was heated in a microwave oven at 180° C. for 10 minutes. The mixture was partitioned between ethyl acetate and saturated NaHCO₃ (30 mL, 1:1). The organic phase was washed with brine (×3), dried (MgSO₄), filtered and concentrated under reduced pressure. The residue was purified by flash chromatography on silica gel column with ethyl acetate/hexane (1/2) to provide 408 mg titled compound (yield 76%).

### EXAMPLE 11B

### N-{3-[2-(4-hydroxyphenoxy)-1,3-thiazol-5-yl]-1-methylprop-2-ynyl}acetamide

A mixture of Example 11A (272 mg, 1.0 mmol), N-(1-methyl-prop-2-ynyl)-acetamide (111 mg, 1.0 mmol), (Ph₃P)₂PdCl₂ (35 mg, 0.05 mmol), CuI (10 mg, 0.05 mmol) and triethylamine (279 μL, 0.2 mmol) in CH₃CN (2.5 mL) was heated in a microwave oven at 90° C. for 10 minutes. It was partitioned between ethyl acetate and brine (50 mL, 1:1). The organic phase was washed with brine, dried (MgSO₄), filtered and concentrated under reduced pressure. The residue was purified by flash chromatography on silica gel column with ethyl acetate to provide 138 mg titled compound (yield 46%).

### EXAMPLE 11C

### N-(3-{2-[4-(cyclopentylmethoxy)phenoxy]-1,3-thiazol-5-yl}-1-methylprop-2-ynyl)acetamide

To a mixture of Example 11B (15 mg, 0.05 mmol), cyclopentyl-methanol (10 mg, 0.1 mmol), and Ph₃P (20 mg, 0.075 mmol) in THF (200 μL) was added DEAD (12 μL, 0.075 mmol) dropwise. The reaction mixture was stirred at room temperature for 30 minutes, and purified by HPLC to provide 14 mg titled compound (yield 74%). ¹H NMR (300 MHz, DMSO-D₆) δ 8.40 (d, J=7.6 Hz, 1H), 7.47 (s, 1H), 7.29 (d, J=9.2 Hz, 2H), 7.01 (d, J=9.2 Hz, 2H), 4.85-4.75 (m, 1H), 3.86 (d, J=6.8 Hz, 2H), 2.36-2.24 (m, 1H), 1.81 (s, 3H), 1.83-1.28 (m, 8H), 1.32 (d, J=7.1 Hz, 3H). MS (ESI) positive ion 385 (M+H)⁺; negative ion 383 (M−H)⁻.

### EXAMPLE 12

### 3-({5-[3-(acetylamino)but-1-ynyl]pyridin-2-yl}oxy)phenyl cyclohexylmethylcarbamate

To a solution of Example 3B (30 mg, 0.1 mmol) and Et₃N (36 uL, 0.25 mmol) in acetonitrile (800 uL) was added cyclohexanemethyl isocyanate (24 uL, 0.15 mmol) at ambient temperature. The reaction was stirred for 30 minutes and became heterogeneous. The mixture was concentrated in vacuo with a rotary evaporator and the crude product was purified by reverse-phase HPLC on an Atlantis C18 column (1.9×10 cm, 5 μm particle size) using a gradient of 5% to 95% acetonitrile: 0.1% aqueous TFA to provide 25 mg (57%) of Example 12 as an amorphous white solid. ¹H NMR (300 MHz, DMSO-D6) δ ppm 0.75-0.98 (m, 2H) 1.06-1.26 (m, 4H) 1.36 (d, J=6.99 Hz, 3H) 1.53-1.76 (m, 5H) 1.83 (s, 3H) 2.90 (t, J=6.25 Hz, 2H) 4.75-5.01 (m, 1H) 5.73 (t, J=5.70 Hz, 1H) 6.89-7.01 (m, 2H) 7.05 (d, J=8.46 Hz, 1H) 7.40 (t, J=8.09 Hz, 1H) 7.79 (t, J=5.88 Hz, 1H) 7.88 (dd, J=8.46, 2.21 Hz, 1H) 8.21 (d, J=2.21 Hz, 1H) 8.43 (d, J=8.09 Hz, 1H); MS (ESI) m/z 436.1 (M+H)⁺.

### EXAMPLE 13

### N-(3-{6-[3-(cyclohexyloxy)phenoxy]pyridin-3-yl}-1-methylprop-2-ynyl)acetamide

Example 13 was prepared according to the procedures described in Example 3 substituting cyclohexanol for cyclohexylmethanol in Example 3C. The crude product was purified by reverse-phase HPLC on an Atlantis C18 column (1.9×10 cm, 5 μm particle size) using a gradient of 5% to 95% acetonitrile: 0.1% aqueous TFA to provide 18 mg (28%) of Example 13 as awhite solid. ¹HNMR (300 MHz, DMSO-D6) δ ppm 1.21-1.56 (m, 9H) 1.62-1.75 (m, 2H) 1.83 (s, 3H) 1.86-1.98 (m, 2H) 4.24-4.40 (m, 1H) 4.73-4.92 (m, 1H) 6.58-6.75 (m, 2H) 6.80 (dd, J=8.09, 2.21 Hz, 1H) 6.99 (d, J=8.46 Hz, 1H) 7.29 (t, J=8.09 Hz, 1H) 7.86 (dd, J=8.46, 2.57 Hz, 1H) 8.21 (d, J=2.21 Hz, 1H) 8.43 (d, J=8.09 Hz, 1H); MS (ESI) m/z 379.1(M+H)⁺.

### EXAMPLE 14

### N-{1-methyl-3-[2-(4-pentylphenoxy-1,3-thiazol-5-yl]prop-2-ynyl}acetamide

### EXAMPLE 14A

### 5-Bromo-2-(4-pentyl-phenoxy)-thiazole

4-Pentyl-phenol (221 mg, 1.35 mmol), 2,5-Dibromo-thiazole (300 mg, 1.2 mmol) and K₂CO₃ (187 mg, 1.35 mmol) were combined in DMSO (3.5 ml). The reaction was placed in the microwave for 30 minutes at 130° C. Upon completion the reaction was poured into water and diluted with EtOAc. The layers were separated and the organics were washed with 15% NaOH and then with water again. The organics were combined, dried (Na₂SO₄), filtered and concentrated by rotary evaporation to provide crude Example 14A which was used without further purification. MS (ESI) m/z 326.4 (M+H⁺); ¹H NMR (300 MHz, d₃-methanol) δ ppm 0.84-0.96 (m, 2H) 1.25-1.41 (m, 3H) 1.48-1.71 (m, 2H) 2.42-2.53 (m, 1H) 2.60-2.68 (m, 2H) 3.25-3.34 (m, 1H) 7.18 (d, J=9.0 Hz, 2H) 7.23 (s, 1H) 7.27 (d, J=9.0 Hz, 2H).

### EXAMPLE 14

### N-{1-methyl-3-[2-(4-pentylphenoxy)-1,3-thiazol-5-yl]prop-2-ynyl}acetamide

Example 14A (392 mg, 1.2 mmol), Example 5B (133 mg, 1.2 mmol), CuI (7.0 mg, 0.036 mmol), Et₃N (507 μg) and THF 3.0 ml were combined in a pressure tube and degassed for 2 minutes with N₂. PdCl₂(PPh₃)₂ (42 mg, 0.06 mmol) was added, the reaction was capped and heated to 80° C. for 30 min. The reaction was cooled, poured into NH₄Cl (sat, aq) and diluted with EtOAc. The layers were separated, the aqueous layer extracted with EtOAc and the combined organics were dried (Na₂SO₄), filtered and concentrated by rotary evaporation. The residue was purified by RP-HPLC to provide Example 14. MS (ESI) m/z 357.1 (M+H⁺); ¹H NMR (300 MHz, DMSO-d₆) δ ppm 0.82-0.91 (m, 2H) 1.24-1.37 (m, 8H) 1.50-1.65 (m, 2H) 1.81 (s, 3H) 2.55-2.64 (m, 2H) 4.75-4.87 (m, 1H) 7.23-7.33 (m, 4H) 7.48 (s, 1H) 8.41 (d, J=7.72 Hz, 1H).

### EXAMPLE 15

### 3-({5-[3-(acetylamino)but-1-ynyl]-1,3-thiazol-2-yl}oxy)phenyl cyclohexylcarbamate

To a solution of Example 1B (30 mg, 0.1 mmol), and Et₃N (36 uL, 0.25 mmol) in acetonitrile (800 uL) was added cyclohexyl isocyanate (25 uL, 0.2 mmol) and the reaction was stirred at ambient temperature for 30 minutes. The mixture was concentrated in vacuo with a rotary evaporator. The crude product was purified by reverse-phase HPLC on an Atlantis C18 column (1.9×10 cm, 5 μm particle size) using a gradient of 5% to 95% acetonitrile: 0.1% aqueous TFA to provide 26 mg (61%) of Example 15 as a white solid. ¹H NMR (300 MHz, DMSO-D6) δ ppm 1.07-1.31 (m, 5H) 1.34 (d, J=6.99 Hz, 3H) 1.50-1.62 (m, 1H) 1.65-1.76 (m, 2H) 1.77-1.90 (m, 5H) 3.24-3.36 (m, J=11.03 Hz, 1H) 4.73-4.91 (m, 1H) 7.03-7.13 (m, 1H) 7.17-7.28 (m, 2H) 7.40-7.56 (m, 2H) 7.80 (d, J=7.72 Hz, 1H) 8.42 (d, J=7.72 Hz, 1H); MS (ESI) m/z 427.9(M+H)⁺.

### EXAMPLE 16

### N-(3-{2-[4-(1-hydroxy-3-methylbutyl)phenoxy]-1,3-thiazol-5-yl}-1-methylprop-2-ynyl)acetamide

### EXAMPLE 16A

### 4-(5-Bromo-thiazol-2-yloxy)-benzaldehyde

A mixture of 2,5-dibromothiazole (3 g, 12.4 mmol), 4-hydroxybenzaldehyde (1.73 g, 14.2 mmol) and potassium carbonate (1.71 g, 12.4 mmol) in DMF (25 mL) was heated in an oil bath at 120° C. for 4 hours. The reaction was cooled to 25° C., poured into water (150 mL) and extracted with 1:1 ethyl acetate/ether (200 mL). The organic layer was washed with water (4×100 mL) and brine (2×100 mL), dried (Na₂SO₄), filtered and evaporated to provide 3.6 g of a residual pale orange oil. The residue was purified by flash chromatography on silica gel eluting with a solvent gradient from 3% to 14% ethyl acetate in hexanes to provide 2 g (57%) of Example 16A as a white solid. ¹H NMR (300 MHz, DMSO-D6) δ ppm 7.53 (s, 1H) 7.58 (d, J=8.46 Hz, 2H) 8.03 (d, J=8.82 Hz, 2H) 10.01 (s, 1H); MS (DCI) m/z 286 (M+H)⁺.

### EXAMPLE 16B

### N-{3-[2-(4-Formyl-phenoxy)-thiazol-5-yl]-1-methyl-prop-2-ynyl}-acetamide

A solution of Example 16A (0.80 g, 2.81 mmol), Example 5B (436 mg, 3.93 mmol), Et₃N (1.17 mL, 8.42 mmol) and THF (20 mL) was degassed and treated with Pd(PPh₃)₂Cl₂ (78.7 mg, 0.11 mmol) and CuI (16 mg, 0.084 mmol). The reaction was heated under nitrogen at 75° C. for 2.5 hours. The solvent was evaporated and the crude concentrate was purified by flash chromatography on silica gel eluting with a solvent gradient from 30% to 60% ethyl acetate in hexanes to provide 550 mg (62%) of Example 16B as a yellow solid. ¹H NMR (300 MHz, DMSO-D6) δ ppm 1.35 (d, J=6.99 Hz, 3H) 1.82 (s, 3H) 4.75-4.93 (m, 1H) 7.56 (s, 1H) 7.60 (d, J=8.82 Hz, 2H) 8.03 (d, J=8.46 Hz, 2H) 8.44 (d, J=8.09 Hz, 1H) 10.01 (s, 1H); MS (ESI) m/z 314.9 (M+H)⁺.

### EXAMPLE 16C

A solution of Example 16B (40 mg, 0.127 mmol) in THF (2 mL), cooled to −78° C., was treated with isobutylmagnesium bromide (2M in THF, 0.127 mL, 0.255 mmol) dropwise over 1 minute and the resulting mixture was stirred at 0° C. for 0.5 hour. Saturated aqueous ammonium chloride (10 mL) was added and the mixture was extracted with CH₂Cl₂ (40 mL). The organic layer was washed with water (10 mL) and brine (10 mL), dried (Na₂SO₄), filtered and evaporated. The residue was purified by reverse-phase HPLC on a Atlantis C18 column (1.9×10 cm, 5 μm particle size) using a gradient of 5% to 100% acetonitrile: 0.1% aqueous TFA to provide 7 mg (15%) of Example 16 as awhite solid. ¹H NMR (300 MHz, DMSO-D6) δ ppm 0.89 (d, J=2.57 Hz, 3H) 0.91 (d, J=2.57 Hz, 3H) 1.28-1.40 (m, 1H) 1.33 (d, J=6.99 Hz, 3H) 1.49-1.61 (m, 1H) 1.62-1.73 (m, 1H) 1.81 (s, 3H) 4.60 (dd, J=8.46, 5.15 Hz, 1H) 4.75-4.91 (m, 1H) 7.30 (d, J=7.35 Hz, 2H) 7.42 (d, J=7.35 Hz, 2H) 7.49 (s, 1H) 8.41 (d, J=7.72 Hz, 1H); MS (ESI) m/z 373.2 (M+H)⁺.

### EXAMPLE 17

### N-{3-[4-(4-isopropoxyphenoxy)phenyl]-1-methylprop-2-ynyl}urea

### EXAMPLE 17A

### 2-{3-[4-(4-Isopropoxy-phenoxy)-phenyl]-1-methyl-prop-2-ynyl}-isoindole-1,3-dione

The desired product was prepared by substituting Example 2B for Example 8A in Example 8B.

### EXAMPLE 17B

### N-{3-[4-(4-isopropoxyphenoxy)phenyl]-1-methylprop-2-ynyl}urea

The desired product was prepared by substituting Example 16A for Example 8B in Example 8C. MS (DCI): m/z 337 (M+H); ¹H NMR (500 MHz, CHLOROFORM-D) δ ppm 1.34 (d, J=6.35 Hz, 6H), 1.50 (d, J=6.35 Hz, 3H), 4.43-4.54 (m, 1H), 4.76 (d, J=6.35 Hz, 1H), 6.82-6.90 (m, 4H), 6.90-7.01 (m, 2H), 7.33 (d, J=8.79 Hz, 2H).

### EXAMPLE 18

### N-{3-[2-(4-{(E)-[(benzyloxy)imino]methyl}phenoxy)-1,3-thiazol-5-yl]-1-methylprop-2-ynyl}acetamide

A solution of Example 16B (40 mg, 0.127 mmol) in MeOH (2 mL) was treated with O-benzylhydroxylamine hydrochloride (24.3 mg, 0.152 mmol). The resulting mixture was heated at 50° C. for 1 hour, cooled to 25° C. and filtered. The filtrate was concentrated by rotary evaporation to give a yellow residue which was purified by reverse-phase HPLC on an Atlantis C18 column (1.9×10 cm, 5 μm particle size) using a gradient of 5% to 100% acetonitrile: 0.1% aqueous TFA to provide 16 mg (30%) of Example 18 as a white solid. ¹H NMR (300 MHz, DMSO-D6) δ ppm 1.33 (d, J=6.99 Hz, 3H) 1.81 (s, 3H) 4.71-4.91 (m, 1H) 5.18 (s, 2H) 7.34-7.40 (m, 5H) 7.42 (d, J=8.82 Hz, 2H) 7.52 (s, 1H) 7.71 (d, J=8.82 Hz, 2H) 8.35 (s, 1H) 8.42 (d, J=8.09 Hz, 1H); MS (ESI) m/z 420.0 (M+H)⁺.

### EXAMPLE 19

### N-(3-{4-[(5-isopropoxypyridin-2-yl)oxy]phenyl}-1-methylprop-2-ynyl)acetamide

### EXAMPLE 19A

### 2-Fluoro-5-isopropoxy-pyridine

To a solution of 2-fluoro-5-hydroxypyridine (6.000 g, 53.05 mmol) in DMF (50 mL) cooled with an ice-bath was added NaH (2.80 g, 60%, 70.0 mml) in several potions. After the addition, the mixture was stirred at 0° C. for 10 minutes. 2-iodopropane (6.5 mL, 65.00 mmol) was added. The mixture was stirred at room temperature for 2 hours. The mixture was poured into water, extracted with ether (2×), the combined extracts were washed with 1N NaOH (1×), water (1×), brine (1×), dried over MgSO₄ and concentrated to give the product that was used directly without further purification.

### EXAMPLE 19B

### 2-(4-Bromo-phenoxy)-5-isopropoxy-pyridine

To a mixture of Example 19A (˜53.05 mmol) and 4-bromophenol (13.80 g, 79.76 mmol) in DMSO (50 mL) cooled with an ice-bath was added NaH (60%, 3.20 g, 80.0 mmol) in several portions. After the addition, the mixture was stirred at room temperature for 10 min. To this was added 5-crown-5 (16.0 mL, 80.6 mmol). The reaction mixture was heated to 160° C. for

### EXAMPLE 19C

### N-(3-{4-[(5-isopropoxypyridin-2-yl)oxy]phenyl}-1-methylprop-2-ynyl)acetamide

The desired product was prepared by substituting Example 19B for Example 2B in Example 2C. MS (DCI): m/z 339 (M+H); ¹H NMR (300 MHz, CHLOROFORM-D) δ ppm 1.34 (d, J=6.25 Hz, 6H), 1.48 (d, J=6.62 Hz, 3H), 2.01 (s, 3H), 4.40-4.58 (m, 1H), 4.95-5.12 (m, 1H), 5.70 (s, 1H), 6.87 (d, J=8.82 Hz, 1H), 6.96-7.06 (m, 2H), 7.28 (dd, J=6.62 Hz, J=8.82 Hz, 1H), 7.36-7.45 (m, 2H), 7.87 (d, J=2.94 Hz, 1H).

### EXAMPLE 20

### N-{1-methyl-3-[6-(4-phenoxyphenoxy)pyridin-3-yl]prop-2-ynyl}acetamide

### EXAMPLE 20A

### 5-bromo-2-(4-phenoxyphenoxy)pyridine

A mixture of 4-Phenoxy-phenol (186 mg, 1.0 mmol), 2,5-dibromo-pyridine (237 mg, 1.0 mmol) and potassium carbonate (138 mg, 1.0 mmol) in DMF (1 mL) was heated in a microwave oven at 200° C. for 30 min. It was partitioned between ethyl acetate and saturated NaHCO₃ (50 mL, 1:1). The organic phase was washed with brine (×3), dried (MgSO₄), filtered and concentrated under reduced pressure to provide 324 mg titled compound (yield 95%).

### EXAMPLE 20B

### N-{1-methyl-3-[6-(4-phenoxyphenoxy)pyridin-3-yl]prop-2-ynyl}acetamide

A mixture of Exmple 20A (34 mg, 0.1 mmol), N-(1-Methyl-prop-2-ynyl)-acetamide (12 mg, 0.11 mmol), (Ph₃P)₂PdCl₂ (3.5 mg, 0.005 mmol), CuI (1 mg, 0.005 mmol) and (28 μL, 0.2 mmol) in CH₃CN (400 μL) was heated in a microwave oven at 100° C. for 20 min. The reaction mixture was filtered and purified by HPLC to get 16 mg titled compound (yield 43%). ¹H NMR (300 MHz, DMSO-D₆) δ 8.43 (d, J=7.5 Hz, 1H), 8.21 (d, J=2.4 Hz, 1H), 7.86 (dd, J1=2.4 Hz, J2=8.8 Hz, 1H), 7.45-7.37 (m, 2H), 7.21-7.01 (m, 8H), 4.85-4.75 (m, 1H), 1.83 (s, 3H), 1.36 (d, J=7.1 Hz, 3H). MS (ESI) positive ion 373 (M+H)⁺; negative ion 371 (M−H)⁻.

### EXAMPLE 21

### N-(3-{2-[4-(cyclohexylmethoxy)phenoxy]-1,3-thiazol-5-yl}-1-methylprop-2-ynyl)acetamide

The titled compound was prepared according to the procedure described in Example 11C substituting cyclohexyl-methanol for cyclopentyl-methanol. ¹H NMR (300 MHz, DMSO-D₆) δ 8.40 (d, J=7.8 Hz, 1H), 7.47 (s, 1H), 7.29 (d, J=9.2 Hz, 2H), 7.01 (d, J=9.2 Hz, 2H), 4.85-4.75 (m, 1H), 3.79 (d, J=6.1 Hz, 2H), 1.81 (s, 3H), 1.86-0.98 (m, 11H), 1.32 (d, J=6.8 Hz, 3H). MS (ESI) positive ion 399 (M+H)⁺; negative ion 397 (M−H)⁻.

### EXAMPLE 22

### N-{3-[6-(3-isobutoxyphenoxy)pyridin-3-yl]-1-methylprop-2-ynyl}acetamide

Example 22 was prepared according to the procedures described in Example 3 substituting isobutanol for cyclohexylmethanol in Example 3C. The crude product was purified by reverse-phase HPLC on an Atlantis C18 column (1.9×10 cm, 5 μm particle size) using a gradient of 5% to 95% acetonitrile: 0.1% aqueous TFA to provide 21 mg (35%) of Example 22 as a white solid. ¹H NMR (300 MHz, DMSO-D6) δ ppm 0.96 (d, J=6.62 Hz, 6H) 1.36 (d, J=6.99 Hz, 3H) 1.83 (s, 3H) 2.00 (d, 1H) 3.73 (d, J=6.62 Hz, 2H) 4.75-4.92 (m, 1H) 6.65-6.75 (m, 2H) 6.76-6.86 (m, 1H) 7.00 (d, J=8.46 Hz, 1H) 7.30 (t, J=7.91 Hz, 1H) 7.86 (dd, J=8.46, 2.57 Hz, 1H) 8.21 (d, J=2.21 Hz, 1H) 8.43 (d, J=8.09 Hz, 1H); MS (ESI) m/z 353.1(M+H)⁺.

### EXAMPLE 23

### N-{3-[2-(3-benzoylphenoxy)-1,3-thiazol-5-yl]-1-methylprop-2-ynyl}acetamide

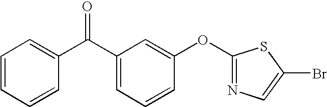

### EXAMPLE 23A

### [3-(5-Bromo-thiazol-2-yloxy)-phenyl]-phenyl-methanone

Example 23A was prepared according to the procedure described in Example 16A substituting 3-hydroxybenzophenone for 4-hydroxybenzaldehyde. The crude product was purified by flash chromatography on silica gel eluting with 5% ethyl acetate/hexanes to provide Example 23A as a colorless oil (82%). ¹H NMR (300 MHz, DMSO-D6) δ ppm 7.48 (s, 1H) 7.58 (t, J=7.54 Hz, 2H) 7.65-7.73 (m, 5H) 7.77 (d, J=6.99 Hz, 2H); MS (DCI) m/z 361.8 (M+H)⁺.

### EXAMPLE 23B

Example 23B was prepared according to the procedure described in Example 16B substituting Example 23A for Example 16A. The crude product was purified by reverse-phase HPLC on an Atlantis C18 column (1.9×10 cm, 5 μm particle size) using a gradient of 5% to 100% acetonitrile: 0.1% aqueous TFA to provide Example 23B. ¹H NMR (300 MHz, DMSO-D6) δ ppm 1.34 (d, J=6.99 Hz, 3H) 1.82 (s, 3H) 4.75-4.91 (m, 1H) 7.52 (s, 1H) 7.58 (t, J=7.35 Hz, 2H) 7.63-7.74 (m, 5H) 7.77 (d, J=6.99 Hz, 2H) 8.43 (d, J=8.09 Hz, 1H); MS (ESI) m/z 391.0 (M+H)⁺.

### EXAMPLE 24

### 3-({5-[3-(acetylamino)but-1-ynyl]-1,3-thiazol-2-yl}oxy)phenyl cyclohexylmethylcarbamate

Example 24 was prepared according to the procedure described in Example 15 substituting cyclohexanemethyl isocyanate for cyclohexyl isocyanate. The crude product was purified by reverse-phase HPLC on an Atlantis C18 column (1.9×10 cm, 5 μm particle size) using a gradient of 5% to 95% acetonitrile: 0.1% aqueous TFA to provide 23 mg (52%) of Example 24 as a white solid. ¹H NMR (300 MHz, DMSO-D6) δ ppm 1.08-1.25 (m, 4H) 1.34 (d, J=6.99 Hz, 3H) 1.42 (dd, J=7.35, 4.04 Hz, 1H) 1.55-1.76 (m, 6H) 1.82 (s, 3H) 2.90 (t, J=6.43 Hz, 2H) 4.73-4.92 (m, 1H) 7.01-7.13 (m, 1H) 7.17-7.28 (m, 2H) 7.39-7.57 (m, 2H) 7.84 (t, J=5.88 Hz, 1H) 8.42 (d, J=7.72 Hz, 1H); MS (ESI) m/z 441.9 (M+H)⁺.

### EXAMPLE 25

### N-(1-methyl-3-{2-[3-(tetrahydro-2H-pyran-2-ylmethoxy)phenoxy]-1,3-thiazol-5-yl}prop-2-ynyl)acetamide

Example 25 was prepared according to the procedures described in Example 3 substituting (Tetrahydro-pyran-2-yl)-methanol for cyclohexylmethanol in Example 3C. The crude product was purified by reverse-phase HPLC on an Atlantis C18 column (1.9×10 cm, 5 μm particle size) using a gradient of 5% to 95% acetonitrile: 0.1% aqueous TFA to provide 14 mg (21%) of Example 25 as a white solid. ¹H NMR (300 MHz, methanol) δ ppm 1.42 (d, J=6.99 Hz, 3H) 1.45-1.73 (m, 5H) 1.87-1.91 (m, 1H) 1.93 (s, 3H) 3.45-3.56 (m, 1H) 3.67-3.75 (m, 1H) 3.94 (d, J=5.15 Hz, 2H) 3.96-4.04 (m, 1H) 4.86-4.95 (m, 1H) 6.81-6.96 (m, 3H) 7.28-7.42 (m, 2H) 8.50 (d, J=7.35 Hz, 1H); MS (ESI) m/z 400.9 (M+H)⁺.

### EXAMPLE 26

### N-(1-methyl-3-{2-[3-(1-phenylethoxy)phenoxy]-1,3-thiazol-5-yl}prop-2-ynyl)acetamide

Example 26 was prepared according to the procedures described in Example 3 substituting 1-phenyl-ethanol for cyclohexylmethanol in Example 3c. The crude product was purified by reverse-phase HPLC on an Atlantis C18 column (1.9×10 cm, 5 μm particle size) using a gradient of 5% to 95% acetonitrile:0.1% aqueous TFA to provide 16 mg (24%) of Example 25 as a white solid. ¹H NMR (300 MHz, methanol) δ ppm 1.43 (d, J=6.99 Hz, 3H) 1.60 (d, J=6.62 Hz, 3H) 1.94 (s, 3H) 4.87-4.99 (m, 1H) 5.39 (q, J=6.50 Hz, 1H) 6.71-6.80 (m, 2H) 6.85 (dd, J=7.54, 1.65 Hz, 1H) 7.15-7.44 (m, 7H) 8.51 (d, J=7.35 Hz, 1H); MS (ESI) m/z 407.1 (M+H)⁺.

### EXAMPLE 27

### N-{3-[4-(4-isopropoxybenzoyl)phenyl]-1-methylprop-2-ynyl}acetamide

### EXAMPLE 27A

### (4-Hydroxy-phenyl)-(4-isopropoxy-phenyl)-methanone

To a mixture of 4,4′-dihydroxybenzophenone (1.185 g, 5.53 mmol), 2-iodopropane (550 μL, 5.50 mmol) in ethanol (2 mL) at refluxing was added a solution of KOH (88%, 370 mg, 5 mmol) in water (2.0 mL) over a period of 10 min. The resulting mixture was refluxed for 1 h. Ethanol was removed. The residue was dissolved in ether, which was washed with brine (1×), dried over MgSO₄ and concentrated. The residue was purified on silica gel eluting with ethyl acetate and hexane to give the desire product as a colorless liquid (439.6 mg).

### EXAMPLE 27B

### Trifluoro-methanesulfonic acid 4-(4-isopropoxy-benzoyl)-phenyl ester

To a mixture of Example 27A (439.6 mg, 1.715 mmol), pyridine (0.80 mL, 9.89 mmol) in methylene chloride (3 mL) cooled with an ice-bath was addedtrifluoromethanesulfonic anhydride (350 μL, 2.080 mmol). After being held for 30 min at 0° C., the mixture was diluted with ether, which was washed with saturated NaHCO₃ (1×), 10% citric acid (2×), brine (1×), dried over MgSO₄ and concentrated to give the desired product as a liquid (648.8 mg, 97.4%), which was used directly without further purification.

### EXAMPLE 27C

### N-{3-[4-(4-isopropoxybenzoyl)phenyl]-1-methylprop-2-ynyl}acetamide

The desired product was prepared by substituting Example 27B for Example 2B in Example 2C. MS(DCI): m/z 350 (M+H); ¹H NMR (500 MHz, CHLOROFORM-D) δ ppm 1.38 (d, J=6.10 Hz, 6H), 1.51 (d, J=6.71 Hz, 3H), 2.02 (s, 3H), 4.66 (heptet, J=6.10 Hz, 1H), 5.02-5.13 (m, 1H), 5.71 (d, J=6.71 Hz, 1H), 6.88-6.96 (m, 2H), 7.47-7.53 (m, 2H), 7.66-7.73 (m, 2H), 7.74-7.80 (m, 2H).

### EXAMPLE 28

### 4-({5-[3-(acetylamino)but-1-ynyl]-1,3-thiazol-2-yl}oxy)phenyl cyclohexylmethylcarbamate

To a solution of Example 81B (30 mg, 0.1 mmol), and Et₃N(36 uL, 0.25 mmol) in acetonitrile (800 uL) was added cyclohexanemethyl isocyanate (25 uL, 0.2 mmol) and the reaction was stirred at ambient temperature for 30 min. The mixture was concentrated in vacuo with a rotary evaporator and the crude product was purified by reverse-phase HPLC on an Atlantis C18 column (1.9×10 cm, 5 μm particle size) using a gradient of 5% to 95% acetonitrile: 0.1% aqueous TFA to provide 26 mg (59%) of Example 28 as a white solid. ¹H NMR (300 MHz, CHLOROFORM-D) δ ppm 0.90-1.05 (m, 2H) 1.14-1.33 (m, 3H) 1.46 (d, J=6.62 Hz, 3H) 1.64-1.83 (m, 6H) 2.00 (s, 3H) 3.11 (t, J=6.43 Hz, 2H) 4.95-5.13 (m, 1H) 7.13-7.32 (m, 5H); MS (ESI) m/z 441.9 (M+H)⁺.

### EXAMPLE 29

### N-{3-[6-(4-isopropoxyphenoxy)pyridin-3-yl]-1-methylprop-2-ynyl}urea

The desired product was prepared by substituting Example 2A for 4-phenoxyphenol in Example 8. MS (DCI): m/z 340 (N+H); ¹H NMR (300 MHz, CHLOROFORM-D) δ ppm 1.35 (d, J=6.25 Hz, 6H), 1.50 (d, J=6.62 Hz, 3H), 4.51 (heptet, J=6.25 Hz, 1H), 4.75-4.88 (m, 1H), 6.75-6.83 (m, 1H), 6.85-6.95 (m, 2H), 6.98-7.06 (m, 2H), 7.65 (dd, J=8.46, 2.21 Hz, 1H), 8.20-8.26 (m, 1H).

### EXAMPLE 30

### N-{3-[2-(3-isobutoxyphenoxy)-1,3-thiazol-5-yl]-1-methylprop-2-ynyl}acetamide

Example 30 was prepared according to the procedures described in Example 1 substituting isobutanol for cyclohexylmethanol in Example 1C. The crude product was purified by reverse-phase HPLC on an Atlantis C18 column (1.9×10 cm, 5 μm particle size) using a gradient of 5% to 95% acetonitrile: 0.1% aqueous TFA to provide 17 mg (32%) of Example 30 as an amorphous white solid. ¹H NMR (300 MHz, DMSO-D6) δ ppm 0.97 (d, J=6.99 Hz, 6H) 1.33 (d, J=6.99 Hz,3H) 1.81 (s, 3H) 1.94-2.09 (m, 1H) 3.76 (d, J=6.62 Hz, 2H) 4.74-4.89 (m, 1H) 6.88-7.00 (m, 3H) 7.37 (t, J=8.27 Hz, 1H) 7.50 (s, 1H) 8.41 (d, J=7.72 Hz, 1H); MS (ESI) m/z 359.1 (M+H)⁺.

### EXAMPLE 31

### N-{1-methyl-3-[4-(3-phenoxyphenoxy)phenyl]prop-2-ynyl}acetamide

The desired product was prepared by substituting 3-phenoxyphenol for Example 1B in Example 2. MS (DCI): m/z 372 (M+H); ¹H NMR (300 MHz, CHLOROFORM-D) δ ppm 1.48 (d, J=6.62 Hz, 3H), 2.00 (none, 2H), 2.01 (s, 3H), 4.96-5.10 (m, 1H), 5.70 (s, 1H), 6.65-6.80 (m, 3H), 6.89-6.98 (m, 2H), 6.99-7.07 (m, 2H), 7.08-7.17 (m, 1H), 7.23-7.28 (m, 1H), 7.28-7.33 (m, 1H), 7.33-7.41 (m, 3H).

### EXAMPLE 32

### N-{3-[6-(4-isopropoxyphenoxy)pyridin-3-yl]-1-methylprop-2-ynyl}acetamide

### EXAMPLE 32A

### 5-Bromo-2-(4-isopropoxy-phenoxy)-pyridine

The desired product was prepared by substituting Example 2A for 4-phenoxyphenol in Example 8A.

### EXAMPLE 32B

### N-{3-[6-(4-isopropoxyphenoxy)pyridin-3-yl]-1-methylprop-2-ynyl}acetamide

The desired product was prepared by substituting Example 32A for Example 2B in Example 2C. MS (DCI): m/z 339 (M+H); ¹H NMR (300 MHz, CHLOROFORM-D) δ ppm 1.35 (d, J=6.25 Hz, 6H), 1.48 (d, J=6.62 Hz, 3H), 2.01 (s, 3H), 4.51 (heptet, J=6.25 Hz, 1H), 4.96-5.13 (m, 1H), 6.79 (d, J=8.46 Hz, 1H), 6.85-6.93 (m, 2H), 6.99-7.08 (m, 2H), 7.65 (dd, J=8.64, 2.39 Hz, 1H), 8.23 (d, J=1.84 Hz, 1H).

### EXAMPLE 33

### 4-({5-[3-(acetylamino)but-1-ynyl]-1,3-thiazol-2-yl}oxy)phenyl cyclohexylcarbamate

Example 33 was prepared according to the procedure described in Example 28 substituting cyclohexyl isocyanate for cyclohexylmethyl isocyanate. The crude product was purified by reverse-phase HPLC on an Atlantis C18 column (1.9×10 cm, 5 μm particle size) using a gradient of 5% to 95% acetonitrile: 0.1% aqueous TFA to provide 24 mg (56%) of Example 33 as an amorphous white solid. 1H NMR (300 MHz, CHLOROFORM-D) δ ppm 1.14-1.43 (m, 6H) 1.45 (d, J=6.99 Hz, 3H) 1.62-1.68 (m, 1H) 1.69-1.82 (m, 2H) 2.00 (s, 3H) 2.01-2.08 (m, 1H) 3.46-3.65 (m, 1H) 4.97-5.10 (m, 1H) 7.12-7.34 (m, 5H); MS (ESI) m/z 427.9 (M+H)⁺.

### EXAMPLE 34

### N-{1-methyl-3-[2-(3-phenoxyphenoxy)-1,3-thiazol-5-yl]prop-2-ynyl}acetamide

The titled compound was prepared according to the procedure described in Example 20, substituting 3-phenoxy-phenol for 4-phenoxy-phenol and 2,5-dibromo-thiazole for 2,5-dibromo-pyridine. ¹H NMR (300 MHz, DMSO-D₆) δ 8.41 (d, J=7.6 Hz, 1H), 7.47-6.91 (m, 10H), 4.83-4.78 (m, 1H), 1.82 (s, 3H), 1.34 (d, J=7.1 Hz, 3H). MS (ESI) positive ion 379 (M+H)⁺; negative ion 377 (M−H)⁻.

### EXAMPLE 35

### N-(1-methyl-3-{6-[3-(tetrahydro-2H-pyran-2-ylmethoxy)phenoxy]pyridin-3-yl}prop-2-ynyl)acetamide

Example 35 was prepared according to the procedures described in Example 3 substituting (Tetrahydro-pyran-2-yl)-methanol for cyclohexylmethanol in Example 3C. The crude product was purified by reverse-phase HPLC on an Atlantis C18 column (1.9×10 cm, 5 μm particle size) using a gradient of 5% to 95% acetonitrile:0.1% aqueous TFA to provide 16 mg (24%) of Example 35 as an amorphous white solid. ¹H NMR (300 MHz, DMSO-D6) δ ppm 1.25-1.33 (m, 1H) 1.36 (d, J=6.99 Hz, 3H) 1.42-1.53 (m, 3H) 1.56-1.68 (m, 1H) 1.75-1.82 (m, 1H) 1.83 (s, 3H) 3.61 (d, 1H) 3.81-3.88 (m, 2H) 3.89 (d, J=4.78 Hz, 2H) 4.71-4.94 (m, 1H) 6.64-6.76 (m, 2H) 6.81 (dd, J=8.09, 1.84 Hz, 1H) 7.00 (d, J=8.46 Hz, 1H) 7.30 (t, J=8.27 Hz, 1H) 7.86 (dd, J=8.46, 2.57 Hz, 1H) 8.21 (d, J=2.57 Hz, 1H) 8.43 (d, J=8.09 Hz, 1H); MS (ESI) m/z 395.1(M+H)⁺.

### EXAMPLE 36

### N-(3-{2-[4-(cyclohexyloxy)phenoxy]-1,3-thiazol-5-yl}-1-methylprop-2-ynyl)acetamide

The titled compound was prepared according to the procedure described in Example 11C substituting cyclohexanol for cyclopentyl-methanol. ¹H NMR (300 MHz, DMSO-D₆) δ 8.37 (d, J=7.8 Hz, 1H), 7.46 (s, 1H), 7.29 (d, J=9.0 Hz, 2H), 7.01 (d, J=9.0 Hz, 2H), 4.85-4.75 (m, 1H), 4.35-4.30 (m, 1H), 1.95-1.25 (m, 10H), 1.81 (s, 3H), 1.32 (d, J=7.2 Hz, 3H). MS (ESI) positive ion 385 (M+H)⁺; negative ion 383 (M−H)⁻.

### EXAMPLE 37

### N-(1-methyl-3-{6-[3-(1-phenylethoxy)phenoxy]pyridin-3-yl}prop-2-ynyl)acetamide

Example 37 was prepared according to the procedures described in Example 3 substituting 1-phenyl-ethanol for cyclohexylmethanol in Example 1c. The crude product was purified by reverse-phase HPLC on an Atlantis C18 column (1.9×10 cm, 5 μm particle size) using a gradient of 5% to 95% acetonitrile: 0.1% aqueous TFA to provide 15 mg (22%) of Example 37 as an amorphous white solid. ¹H NMR (300 MHz, DMSO-D6) δ ppm 1.36 (d, J=6.99 Hz, 3H) 1.53 (d, J=6.25 Hz, 3H) 1.83 (s, 3H) 4.74-4.93 (m, 1H) 5.50 (q, J=6.50 Hz, 1H) 6.56-6.72 (m, 2H) 6.77 (dd, J=7.91, 2.02 Hz, 1H) 6.93 (d, J=8.46 Hz, 1H) 7.13-7.45 (m, 6H) 7.83 (dd, J=8.64, 2.39 Hz, 1H) 8.17 (d, J=2.57 Hz, 1H) 8.43 (d, J=8.09 Hz, 1H); MS (ESI) m/z 401.1 (M+H)⁺.

### EXAMPLE 38

### N-{3-[4-(4-isopropoxybenzyl)phenyl]-1-methylprop-2-ynyl}acetamide

The desired product was prepared by substituting bis(4-hydroxyphenyl)methane for 4,4′-dihydroxybenzophenone in Example 27. MS (DCI): m/z 336 (M+H); ¹H NMR (300 MHz, CHLOROFORM-D) δ ppm 1.32 (d, J=6.25 Hz, 6H), 1.47 (d, J=6.99 Hz, 3H), 2.00 (s, 3H), 3.89 (s, 2H), 4.50 (heptet, J=6.25 Hz, 1H), 4.95-5.09 (m, 1H), 5.68 (br. s, 1H), 6.75-6.85 (m, 2H), 6.99-7.08 (m, 2H), 7.08-7.15 (m, J=8.46 Hz, 2H), 7.28-7.36 (m, J=8.09 Hz, 2H).

### EXAMPLE 39

### N-{3-[2-(3-isopropoxyphenoxy)-1,3-thiazol-5-yl]-1-methylprop-2-ynyl}acetamide

Example 39 was prepared according to the procedures described in Example 1 substituting isopropanol for cyclohexylmethanol in Example 1C. The crude product was purified by reverse-phase HPLC on an Atlantis C18 column (1.9×10 cm, 5 μm particle size) using a gradient of 5% to 95% acetonitrile: 0.1% aqueous TFA to provide 18 mg (39%) of Example 39 as an amorphous white solid. ¹H NMR (300 MHz, methanol) δ ppm 1.31 (d, J=6.25 Hz, 6H) 1.42 (d, J=6.99 Hz, 3H) 1.93 (s, 3H) 4.53-4.68 (m, 1H) 4.86-4.95 (m, 1H) 6.77-6.92 (m, 3H) 7.28-7.39 (m, 2H); MS (ESI) m/z 345.1 (M+H)⁺.

### EXAMPLE 40

### 4-({5-[3-(acetylamino)but-1-ynyl]-1,3-thiazol-2-yl}oxy)phenyl 2-phenylethylcarbamate

Example 40 was prepared according to the procedure described in Example 28 substituting 2-phenethyl isocyanate for cyclohexanemethyl isocyanate. The crude product was purified by reverse-phase HPLC on an Atlantis C18 column (1.9×10 cm, 5 μm particle size) using a gradient of 5% to 95% acetonitrile:0.1% aqueous TFA to provide 27 mg (45%) of Example 40 as an amorphous white solid. ¹H NMR (300 MHz, DMSO-D6) δ ppm 1.33 (d, J=6.99 Hz, 3H) 1.82 (s, 3H) 2.79 (t, J=7.35 Hz, 2H) 3.20-3.37 (m, 2H) 4.73-4.91 (m, 1H) 7.12-7.42 (m, 9H) 7.49 (s, 1H) 7.91 (t, J=5.52 Hz, 1H) 8.42 (d, J=7.72 Hz, 1H); MS (ESI) m/z 450.1 (M+H)⁺.

### EXAMPLE 41

### 4-({5-[3-(acetylamino)but-1-ynyl]-1,3-thiazol-2-yl}oxy)phenyl cyclopentylcarbamate

Example 41 was prepared according to the procedure described in Example 28 substituting cyclopentyl isocyanate for cyclohexanemethyl isocyanate. The crude product was purified by reverse-phase HPLC on an Atlantis C18 column (1.9×10 cm, 5 μm particle size) using a gradient of 5% to 95% acetonitrile:0.1% aqueous TFA to provide 21 mg (38%) of Example 41 as an amorphous white solid. ¹H NMR (300 MHz, DMSO-D6) δ ppm 1.33 (d, J=6.99 Hz, 3H) 1.42-1.57 (m, 4H) 1.60-1.73 (m, 2H) 1.75-1.92 (m, 5H) 3.74-3.91 (m, 1H) 4.74-4.89 (m, 1H) 7.16-7.25 (m, 2H) 7.32-7.41 (m, 2H) 7.49 (s, 1H) 7.84 (d, J=7.35 Hz, 1H) 8.42 (d, J=8.09 Hz, 1H); MS (ESI) m/z 414.1(M+H)⁺.

### EXAMPLE 42

### N-{3-[4-(4-isopropoxyphenoxy)phenyl]prop-2-ynyl}acetamide

The desired product was prepared by substituting N-prop-2-ynyl-acetamide for N-(1-methyl-prop-2-ynyl)-acetamide in Example 2. MS (DCI): m/z 324 (M+H); ¹H NMR (300 MHz, CHLOROFORM-D) δ ppm 1.34 (d, J=5.88 Hz, 6H), 2.03 (s, 3H), 4.26 (d, J=5.15 Hz, 2H), 4.49(heptet, J=5.88 Hz, 1H), 5.66 (s, 1H), 6.79-6.92 (m, 4H), 6.92-7.00 (m, 2H), 7.29-7.39 (m, 2H).

### EXAMPLE 43

### N-{3-[6-(3-isopropoxyphenoxy)pyridin-3-yl]-1-methylprop-2-ynyl}acetamide

The desired product was prepared by substituting 3-isopropoxyphenol for 4-isopropoxyphenol in Example 32. MS (DCI): m/z 339 (M+H); ¹H NMR (300 MHz, CHLOROFORM-D) δ ppm 1.33 (d, J=5.88 Hz, 6H), 1.48 (d, J=6.62 Hz, 3H), 2.01 (s, 3H), 4.51 (heptet, J=5.88 Hz, 1H), 4.93-5.15 (m, 1H), 5.69 (br s, 1H), 6.62-6.70 (m, 2H), 6.74 (dd, J=8.46, 1.84 Hz, 1H), 6.83 (d, J=8.46 Hz, 1H), 7.67 (dd, J=8.46, 2.21 Hz, 1H), 8.25 (d, J=1.84 Hz, 1H).

### EXAMPLE 44

### 3-({5-[3-(acetylamino)but-1-ynyl]-1,3-thiazol-2-yl}oxy)-N-(cyclohexylmethyl)benzamide

### EXAMPLE 44A

### 3-(5-Bromo-thiazol-2-yloxy)-benzoic acid

A solution of Example 58A (2.02 g, 6.18 mmol) in EtOH (30 mL) was treated with 2M LiOH (7 mL). The reaction was stirred at 25° C. for 1.5 h and was concentrated to a volume of ˜7 mL on a rotary evaporator. The aqueous residue was diluted with water (15 mL), acidified to pH 2 with 1N HCl, and extracted with EtOAc (2×60 mL). The organic layers were washed with brine (20 mL), dried (Na₂SO₄) and concentrated to provide 1.70 g (92%) of Example 44A as a white solid. ¹H NMR (300 MHz, DMSO-D6) δ ppm 13.31 (br s, 1H) 7.88 (dd, J=6.07, 2.76 Hz, 1H) 7.84 (s, 1H) 7.57-7.68 (m, 2H) 7.47 (s, 1H); MS (DCI) m/z 300 (M+H)⁺.

### EXAMPLE 44B

### 3-(5-Bromo-thiazol-2-yloxy)-benzoyl chloride

A solution of Example 44A (350 mg, 1.17 mmol) in CHCl₃ (10 mL), cooled to 0° C., was treated with DMF (30 μL, 0.39 mmol). Oxalyl chloride (205.5 μL, 2.34 mmol) was added to the reaction over 2 min and the resulting solution was stirred at 0° C. for 10 min and at 25° C. for 1h. The reaction mixture was concentrated on a rotary evaporator and dried to constant weight to provide 385 mg Example 44B.

### EXAMPLE 44C

To a solution of Example 44B (60 mg, 0.19 mmol), and triethylamine (79 uL, 0.57 mmol) in methylene chloride (1 mL) was added cyclohexanemethylamine (27 uL, 0.21 mmol). The reaction was stirred for 4 h at ambient temperature and diluted with methylene chloride (30 mL). The mixture was washed with 1N HCl (15 mL) and brine (10 mL), dried over sodium sulfate, filtered, and concentrated to provide 72 mg (96%) of Example 44C. 1H NMR (300 MHz, DMSO-D6) δ ppm 0.82-1.03 (m, 2H) 1.07-1.29 (m, 3H) 1.46-1.78 (m, 6H) 3.10 (t, J=6.25 Hz, 2H) 7.47 (s, 1H) 7.49-7.65 (m, 2H) 7.74-7.88 (m, 2H) 8.54 (t, J=5.70 Hz, 1H); MS (ESI) m/z 396.9 (M+H)⁺.

### EXAMPLE 44D

A solution of Example 44C (62 mg, 0.16 mmol), Example 5B (25 mg, 0.22 mmol), and Et₃N (68 μL, 0.66 mmol) in THF (15 mL) was degassed and treated with Pd(PPh₃)₂Cl₂ (6 mg, 0.008 mmol) and CuI (0.8 mg, 0.004 mmol). The reaction was heated under nitrogen at 80° C. for 2.5 h. The solvent was evaporated and the crude concentrate was purified by reverse-phase HPLC on an Atlantis C18 column (1.9×10 cm, 5 μm particle size) using a gradient of 5% to 95% acetonitrile:0.1% aqueous TFA-to provide 27 mg (40%) of Example 44D as an off-white solid. ¹H NMR (300 MHz, DMSO-D6) δ ppm 0.81-1.02 (m, 2H) 1.10-1.26 (m, 3H) 1.34 (d, J=6.99 Hz,3H) 1.47-1.78 (m, 6H) 1.82 (s, 3H) 3.10 (t, J=6.43 Hz, 2H) 4.75-4.91 (m, 1H) 7.47-7.64 (m, 3H) 7.78-7.87 (m, 2H) 8.43 (d, J=7.72 Hz, 1H) 8.53 (t, J=5.52 Hz, 1H); MS (ESI) m/z 456.0 (M+H)⁺.

### EXAMPLE 45

### 4-({5-[3-(acetylamino)but-1-ynyl]-1,3-thiazol-2-yl}oxy)phenyl butylcarbamate

Example 45 was prepared according to the procedure described in Example 28 substituting n-butyl isocyanate for cyclohexylmethyl isocyanate. The crude product was purified by reverse-phase HPLC on an Atlantis C18 column (1.9×10 cm, 5 μm particle size) using a gradient of 5% to 95% acetonitrile:0.1% aqueous TFA to provide 18 mg (33%) of Example 45 as an amorphous white solid. ¹H NMR (300 MHz, DMSO-D6) δ ppm 0.89 (t, J=7.17 Hz, 3H) 1.24-1.38 (m, 5H) 1.38-1.51 (m, 2H) 1.81 (s, 3H) 3.00-3.12 (m, 2H) 4.71-4.91 (m, 1H) 7.14-7.25 (m, 2H) 7.31-7.42 (m, 2H) 7.49 (s, 1H) 7.80 (t, J=5.88 Hz, 1 H) 8.42 (d, J=8.09 Hz, 1H); MS (ESI) m/z 402.1 (M+H)⁺.

### EXAMPLE 46

### N-{1-methyl-3-[2-(4-propoxyphenoxy)-1,3-thiazol-5-yl]prop-2-ynyl}acetamide

The titled compound was prepared according to the procedure described in Example 11C substituting propan-1-ol for cyclopentyl-methanol. ¹H NMR (300 MHz, DMSO-D₆) δ 8.40 (d, J=8.1 Hz, 1H), 7.47 (s, 1H), 7.30 (d, J=9.2 Hz, 2H), 7.01 (d, J=9.2 Hz, 2H), 4.85-4.75 (m, 1H), 3.94 (t, J=6.6 Hz, 2H), 1.81 (s, 3H), 1.83-1.68 (m, 2H), 1.32 (d, J=7.1 Hz, 3H), 0.98 (t, J=7.5 Hz, 3H). MS (ESI) positive ion 345 (M+H)⁺; negative ion 343 (M−H)⁻.

### EXAMPLE 47

### N-(3-{2-[4-(anilinomethyl)phenoxy]-1,3-thiazol-5-yl}-1-methylprop-2-ynyl)acetamide

Example 16B (152 mg, 0.48 mmol) was combined with aniline (46 μl, 0.50 mmol) in 4 ml of buffer solution (composed of 6 g NaOAc, 8.5 ml HOAc and 250 ml MeOH. This solution was prepared and stored for use as solvent for reductive amination reactions). To the reaction solution was added sodium triacetoxyborohydride (305 mg, 1.44 mmol) and the reaction was stirred for 16 h. At that time, the reaction was still incomplete so an additional 0.50 mmol aniline and 1.44 mmol sodium triacetoxyborohydride were added and the reaction was stirred an additional 4 h, quenched with water and overwhelmed with EtOAc. The layers were separated, the aqueous portion was extracted with EtOAc and the combined organics were dried (Na₂SO₄), filtered and concentrated by rotary evaporation. The residue was purified by RP-HPLC to provide the title compound. MS (ESI) m/z 392.0 (M+H⁺); ¹H NMR (300 MHz, DMSO-d₆) δ ppm 1.32 (d, J=6.99 Hz, 3H) 1.81 (s, 3H) 4.29 (s, 2H) 4.75-4.87 (m, 1H) 6.50-6.63 (m, 2H) 7.05 (t, J=7.91 Hz, 1H) 7.32 (d, J=8.82 Hz, 2H) 7.40-7.50 (m, 4H) 7.52-7.67 (m, 1H) 7.99-8.06 (m, 1H) 8.41 (d, J=7.35 Hz, 1H).

### EXAMPLE 48

A solution of Example 62 (35 mg, 0.09 mmol) in EtOH (1.5 mL) and CH₂Cl₂ (0.5 mL) was treated with NaBH₄ (10.2 mg, 0.27 mmol) portionwise over 2 min and the reaction was stirred at 25° C. for 0.5 h. Water (5 mL) was added and the resulting mixture was extracted with EtOAc (25 mL). The organic phase was washed with brine (5 mL), dried over Na₂SO₄, and concentrated. The crude product was purified by reverse-phase HPLC on an Atlantis C18 column (1.9×10 cm, 5 μm particle size) using a gradient of 5% to 100% acetonitrile:0.1% aqueous TFA to provide 9 mg (30%) of Example 48 as a white solid. ¹H NMR (300 MHz, DMSO-D6) δ ppm 8.40 (d, J=7.72 Hz, 1H) 7.46 (s, 1H) 7.46 (d, J=8.82 Hz, 2H) 7.39 (d, J=6.99 Hz, 2H) 7.31 (t, J=7.35 Hz, 2H) 7.29 (d, J=8.82 Hz, 2H) 7.21 (none, 1H) 7.21 (t, J=7.17 Hz, 1H) 5.74 (s, 1H) 4.77 (none, 1H) 4.58-5.00 (m, 1H) 1.81 (s, 3H) 1.32 (d, J=6.99 Hz, 3H); MS (ESI) m/z 393.1 (M+H)⁺.

### EXAMPLE 49

### 4-[2-(4-anilinophenoxy)-1,3-thiazol-5-yl]but-3-yn-2-ol

### EXAMPLE 49A

A 5 mL microwave reaction tube was charged with 2,5-dibromothiazole (61 mg, 0.25 mmol), 4-phenylaminophenol (46 mg, 0.25 mmol), potassium carbonate (103 mg, 0.75 mmol) and dmf (1 mL). The tube was sealed and microwaved at 180° C. for 5 min. LC/MS of an aliquot in methanol showed a complete reaction. The reaction mixture was poured into water and extracted with ethyl acetate. The combined organic layers were back-extracted with brine and dried over sodium sulfate, filtered and concentrated. The crude material was purified on a 5000 mg pre-packed silica gel column (7.5% EtOAc/Hexane) to provide 55 mg (63%) of Example 49A. Mass spec (ESD) 348.7 (M+H), 344.8 (M−H). ¹H NMR (δ, DMSO):8.32 (s, 1H), 7.42 (s, 1H), 7.28-7.23 (m, 4H), 7.14-7.07 (m, 4H), 6.86 (t, 1H).

### EXAMPLE 49B

A 5 mL microwave reaction tube was charged with Example 49A (50 mg, 0.143 mmol), 3-butyn-2-ol (16.8 μL, 0.172 mmol), diisopropylamine (24.4 μL, 0.172 mmol), copper (I) iodide (0.3 mg, 0.0014 mmol), triphenylphosphine (0.4 mg, 0.0016 mmol), bis(acetonitrile)dichloropalladium (II) (0.2 mg, 0.0007 mmol) and dmf (0.5 mL). The tube was sealed and microwaved at 120° C. for 20 min. LC/MS of an aliquot in methanol showed a complete reaction. The reaction mixture was filtered through a small plug of silica gel using acetonitrile and concentrated. The crude material was purified on a 5000 mg pre-packed silica gel column (20% EtOAc/Hexane) to provide 15 mg (33%) of Example 49B. Mass spec (ESI): 336.9 (M+H), 334.9 (M−H). ¹H NMR (δ, DMSO):8.31 (s, 1H), 7.48 (s, 1H), 7.28-7.23 (m, 4H), 7.13-7.07 (m, 4H), 6.86 (t, 1H), 5.51 (d, 1H), 4.8 (m, 1H), 1.35 (d, 3H).

### EXAMPLE 50

### N-[3-(2-{4-[(E)-(hydroxyimino)(phenyl)methyl]phenoxy}-1,3-thiazol-5-yl)-1-methylprop-2-ynyl]acetamide

A solution of Example 62 (40 mg, 0.103 mmol) in MeOH (2 mL) was treated with pyridine (16.6 μL, 0.206 mmol) and hydroxylamine hydrochloride (10.7 mg, 0.154 mmol). The resulting mixture was heated at 60° C. for 15 h and was concentrated by rotary evaporation. The concentrate was purified by reverse-phase HPLC on an Atlantis C18 column (1.9×10 cm, 5 μm particle size) using a gradient of 5% to 100% acetonitrile:0.1% aqueous TFA to provide 16 mg (38%) of Example 50 as a white solid as a 1:1 mixture of E/Z isomers. ¹H NMR (300 MHz, DMSO-D6) δ ppm 11.47, 11.43 (s, 1H) 8.44, 8.41 (d, J=2.94 Hz, d, J=3.31 Hz, 1H) 7.27-7.55 (m, 10H) 4.66-4.94 (m, 1H) 1.82, 1.81 (s, 1H) 1.35, 1.33 (d, J=2.94 Hz, d, J=2.94 Hz, 3H); MS (ESI) m/z 406.1 (M+H)⁺.

### EXAMPLE 51

### N-(3-{4-[(2-isopropoxypyrimidin-4-yl)oxy]phenyl}-1-methylprop-2-ynyl)acetamide

### EXAMPLE 51A

### 4-(4-Bromo-phenoxy)-2-chloro-pyrimidine

A mixture of 4-bromophenol (1.161 g, 6.712 mmol), NaOH (1.0 N, 6.7 mL, 6.70 mmol) in acetone/water (15 mL/15 mL) was stirred at room temperature for 20 min. Then 2,4-dichloropyrimidine (1.00 g, 6.712 mmol) was added. The reaction mixture was stirred at room temperature for 3 hours. Acetone was removed under vacuum. The residue was diluted with water, the solid was filtered, washed with water and dried under vacuum at ˜50° C. to give the desired product as an off-white solid (1.6976 g, 88.6%).

### EXAMPLE 51B

### 4-(4-Bromo-phenoxy)-2-isopropoxy-pyrimidine

To a solution of NaH (60%, 28 mg, 0.688 mmol) in DMF (1.5 mL) at ° C. was added isopropanol (43 μL, 0.555 mmol). After 30 mim, Example 51A (143 mg 0.500 mmol) was added. The mixture was stirred at room temperature overnight. The mixture was poured into water, extracted with ether (1×). The ether layer was washed with brine (1×), dried over MgSO₄ and concentrated. The residue was purified on silica gel eluting with ethyl acetate and hexane to give the desired product as a white solid (114.9 mg, 74.4%).

### EXAMPLE 51C

### N-(3-{4-[(2-isopropoxypyrimidin-4-yl)oxy]phenyl}-1-methylprop-2-ynyl)acetamide

The desired product was prepared by substituting Example 51B for Example 2B in Example 2C. MS (DCI): m/z 340 (N+H); ¹H NMR (300 MHz, CHLOROFORM-D) δ ppm 1.31 (d, J=5.88 Hz, 6H), 1.49 (d, J=6.99 Hz, 3H), 2.02 (s, 3H), 4.97-5.12 (m, 1H), 5.23 (heptet, J=5.88 Hz, 1H), 5.69 (s, 1H), 6.39 (d, J=5.88 Hz, 1H), 7.14 (d, J=8.82 Hz, 2H), 7.45 (d, J=8.46 Hz, 2H), 8.17 (d, J=5.88 Hz, 1H).

### EXAMPLE 52

### N-(3-{2-[4-(isobutylamino)phenoxy]-1,3-thiazol-5-yl}-1-methylprop-2-ynyl)acetamide

The titled compound was prepared according to the procedure described in Example 34, substituting 4-isobutylamino-phenol for 3-phenoxy-phenol. ¹H NMR (300 MHz, DMSO-D₆) δ 8.39 (d, J=7.8 Hz, 1H), 7.45 (s, 1H), 7.07 (d, J=9.2 Hz, 2H), 6.60 (d, J=9.2 Hz, 2H), 5.88 (t, J=6.1 Hz, 1H), 4.85-4.75 (m, 1H), 2.82 (t, J=6.1 Hz, 2H), 1.90-1.78 (m, 1H), 1.80 (s, 3H), 1.31 (d, J=7.1 Hz, 3H), 0.94 (d, J=6.5 Hz, 6H), MS (ESI) positive ion 358 (M+H)⁺; negative ion 356(M−H)⁻.

### EXAMPLE 53

### 3-({5-[3-(acetylamino)but-1-ynyl]pyridin-2-yl}oxy)phenyl cyclohexylcarbamate

Example 53 was prepared according to the procedure described in Example 12 substituting cyclohexyl isocyanate for cyclohexanemethyl isocyanate. The crude product was purified by reverse-phase HPLC on an Atlantis C18 column (1.9×10 cm, 5 μm particle size) using a gradient of 5% to 95% acetonitrile:0.1% aqueous TFA to provide 22 mg (52%) of Example 53 as a white solid. ¹H NMR (300 MHz, DMSO-D6) δ ppm 1.08-1.31 (m, 5H) 1.36 (d, J=6.99 Hz, 3H) 1.48-1.60 (m, 1H) 1.64-1.75 (m, 2H) 1.74-1.93 (m, 5H) 3.24-3.33 (m, 1H) 4.75-4.93 (m, 1H) 6.89-7.02 (m, 2H) 7.05 (d, J=8.46 Hz, 1H) 7.40 (t, J=8.09 Hz, 1H) 7.74 (d, J=7.72 Hz, 1H) 7.88 (dd, J=8.46, 2.21 Hz, 1H) 8.21 (d, J=2.57 Hz, 1H) 8.43 (d, J=7.72 Hz, 1H); MS (ESI) m/z 422.1 (M+H)⁺.

### EXAMPLE 54

### tert-butyl 4-({5-[3-(acetylamino)but-1-ynyl]-1,3-thiazol-2-yl}oxy)phenylcarbamate

### EXAMPLE 54A

### [4-(5-Bromo-thiazol-2-yloxy)-phenyl]-carbamic acid tert-butyl ester

Example 54A was prepared in the exact same manner as Example 14A substituting (4-Hydroxy-phenyl)-carbamic acid tert-butyl ester for 4-Pentyl-phenol. MS (ESI) m/z 371.0 (M+H+); ¹H NMR (300 MHz, DMSO-d₆) δ ppm 1.48 (s, 9H) 7.28 (d, J=9.19 Hz, 2H) 7.42 (s, 1H) 7.54 (d, J=8.82 Hz, 2H) 9.52 (s, 1H).

### EXAMPLE 54

### tert-butyl 4-({5-[3-(acetylamino)but-1-yl]-1,3-thiazol-2-yl}oxy)phenylcarbamate

Example 54 was prepared in the exact same manner as 14, substituting Example 54A for 14A. MS (ESI) m/z 402.0 (M+H⁺); ¹H NMR (300 MHz, DMSO-d₆) δ ppm 1.32 (d, J=6.99 Hz, 3H) 1.44-1.52 (m, 9H) 1.81 (s, 3H) 4.74-4.87 (m, 1H) 7.27 (d, J=9.0 Hz, 2H) 7.48 (s, 1H) 7.53 (d, J=9.0 Hz, 2H) 8.41 (d, J=7.72 Hz, 1H) 9.52 (s, 1H).

### EXAMPLE 55

### N-(3-{2-[4-(benzylamino)phenoxy]-1,3-thiazol-5-yl}-1-methylprop-2-ynyl)acetamide

### EXAMPLE 55A

### Benzyl-[4-(5-bromo-thiazol-2-yloxy)-phenyl]-amine

Example 55A was prepared in the exact same manner as Example 14A, substituting 4-Benzylamino-phenol for 4-Pentyl-phenol. The crude material was used in the next step without further purification.

### EXAMPLE 55

### N-(3-{2-[4-(benzylamino)phenoxy]-1,3-thiazol-5-yl}-1-methylprop-2-ynyl)acetamide

Example 55 was prepared in the exact same manner as Example 14, substituting Example 55A for Example 14A. MS (ESI) m/z 390.2 (M+H⁺); ¹H NMR (300 MHz, DMSO-d₆) δ ppm 1.27-1.37 (m, 3H) 1.76-1.84 (m, 3H) 4.27 (d, J=5.88 Hz, 2H) 4.74-4.88 (m, 1H) 6.50 (t, J=5.88 Hz, 1H) 6.58-6.65 (m, 1H) 7.02-7.09 (m, 1H) 7.28-7.47 (m, 3H) 7.50-7.58 (m, 3H) 7.95 (dd, J=7.17, 2.39 Hz, 1H) 8.36-8.45 (m, 1H) 8.66 (s, 1H).

### EXAMPLE 56

### N-{3-[6-(4-isopropylphenoxy)pyridin-3-yl]-1-methylprop-2-ynyl}acetamide

### EXAMPLE 56A

### 5-Bromo-2-(4-isopropyl-phenoxy)-pyridine

Example 56A was prepared in the exact same manner as Example 7A, substituting 4-Isopropyl-phenol for 4-Pentyl-phenol to provide the title compound. MS (ESI) m/z 294.5 (M+H⁺); ¹H NMR (300 MHz, DMSO-d₆) δ ppm 1.22 (d, J=6.99 Hz, 6H) 2.83-2.98 (m, 1H) 7.01 (d, J=8.82 Hz, 1H) 7.06 (d, J=9.0 Hz, 2H) 7.27 (d, J=9.0 Hz, 2H) 8.02 (d, J=2.57 Hz, 1H) 8.05 (d, J=2.57 Hz, 1H) 8.26 (d, J=1.84 Hz, 1H).

### EXAMPLE 56

### N-{3-[6-(4-isopropylphenoxy)pyridin-3-yl]-1-methylprop-2-ynyl}acetamide

Example 56 was prepared in the exact same manner as Example 7, substituting Example 56A for 7A. MS (ESI) m/z 323.7 (M+H⁺); ¹H NMR (300 MHz, DMSO-d₆) δ ppm 1.22 (d, J=6.99 Hz, 6H) 1.36 (d, J=6.99 Hz, 3H) 1.83 (s, 3H) 2.83-2.97 (m, 1H) 4.78-4.89 (m, 1H) 7.05 (d, J=8.46 Hz, 2H) 7.29 (d, J=8.46 Hz, 2H) 7.83 (d, J=2.21 Hz, 1H) 7.86 (d, J=2.57 Hz, 1H) 8.18 (d, J=1.84 Hz, 1H) 8.43 (d, J=8.09 Hz, 1H).

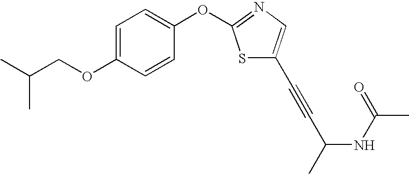

### EXAMPLE 57

### N-{3-[2-(4-isobutoxyphenoxy)-1,3-thiazol-5-yl]-1-methylprop-2-ynyl}acetamide

The titled compound was prepared according to the procedure described in Example 11C substituting 2-methyl-propan-1-ol for cyclopentyl-methanol. ¹H NMR (300 MHz, DMSO-D₆) δ 8.40 (d, J=7.6 Hz, 1H), 7.47 (s, 1H), 7.30 (d, J=9.1 Hz, 2H), 7.01 (d, J=9.1 Hz, 2H), 4.85-4.75 (m, 1H), 3.76 (d, J=6.4 Hz, 2H), 2.06-1.97 (m, 1H), 1.81 (s, 3H), 1.32 (d, J=7.0 Hz, 3H), 0.99 (d, J=6.7 Hz, 6H), MS (ESI) positive ion 359 (M+H)⁺; negative ion 357(M−H)⁻.

### EXAMPLE 58

### ethyl 3-({5-[3-(acetylamino)but-1-ynyl]-1,3-thiazol-2-yl}oxy)benzoate

### EXAMPLE 58A

### 3-(5-Bromo-thiazol-2-yloxy)-benzoic acid ethyl ester

Example 58A was prepared according to the procedure described in Example 16A substituting 3-hydroxyethylbenzoate for 4-hydroxybenzaldehyde. The crude product was purified by flash chromatography on silica gel eluting with a solvent gradient from 1% to 10% EtOAc in hexanes to provide Example 58A as a colorless oil (62%).

1H NMR (300 MHz, DMSO-D6) δ ppm 1.32 (t, J=6.99 Hz, 3H) 4.33 (q, J=6.99 Hz, 2H) 7.47 (s, 1H) 7.61-7.71 (m, 2H) 7.84-7.96 (m, 2H); MS (ESI) m/z 329.7 (M+H)⁺.

### EXAMPLE 58B

Example 58B was prepared according to the procedure described in Example 16B substituting Example 58A for Example 16A. The crude product was purified by flash chromatography on silica gel eluting with a solvent gradient from 10% to 50% EtOAc in hexanes to provide Example 58b (49%). ¹H NMR (300 MHz, DMSO-D6) δ ppm 1.28-1.37 (m, 6H) 1.82 (s, 3H) 4.33 (q, J=7.23 Hz, 2H) 4.67-4.96 (m, 1H) 7.52 (s, 1H) 7.59-7.75 (m, 2H) 7.82-8.00 (m, 2H) 8.43 (d, J=7.72 Hz, 1H); MS (ESI) m/z 359.0 (M+H)⁺.

### EXAMPLE 59

### N-{3-[2-(3-anilinophenoxy)-1,3-thiazol-5-yl]-1-methylprop-2-ynyl}acetamide

The titled compound was prepared according to the procedure described in Example 34, substituting 3-phenylamino-phenol for 3-phenoxy-phenol. ¹H NMR (300 MHz, DMSO-D₆) δ 8.44 (s, 1H), 8.40 (d, J=8.1 Hz, 1H), 7.51 (s, 1H), 7.35-6.72 (m, 9H), 4.85-4.78 (m, 1H), 1.81 (s, 3H), 1.33 (d, J=7.1 Hz, 3H). MS (ESI) positive ion 378 (M+H)⁺; negative ion 376 (M−H)⁻.

### EXAMPLE 60

### N-{3-[2-(4-cyclopentylphenoxy)-1,3-thiazol-5-yl]-1-methylprop-2-ynyl}acetamide

### EXAMPLE 60A

### 5-Bromo-2-(4-cyclopentyl-phenoxy)-thiazole

Example 60A was prepared in the exact same manner as 14A, substituting 4-Cyclopentyl-phenol for 4-Pentyl-phenol to give Example 60A which was used in the next step without further purification. MS (ESI) m/z 324.1 (M+H⁺); ¹H NMR (300 MHz, methanol) δ ppm 1.42-2.15 (m, 8H) 2.96-3.11 (m, 1H) 7.16-7.20 (m, 2H) 7.23 (s, 1H) 7.32-7.36 (m, 2H).

### EXAMPLE 60

### N-{3-[2-(4-cyclopentylphenoxy)-1,3-thiazol-5-yl]-1-methylprop-2-ynyl}acetamide

Example 60 was prepared in the exact same manner as Example 14, substituting Example 60A for Example 14A. MS (ESI) m/z 315.1 (M+H⁺);

### EXAMPLE 61

### N-{3-[2-(4-{[(4-tert-butylphenyl)amino]methyl}phenoxy)-1,3-thiazol-5-yl]-1-methylprop-2-ynyl}acetamide

Example 61 was prepared in the exact same manner as Example 47, substituting 4-tert-Butyl-phenylamine for aniline. MS (ESI) m/z 448.2 (M+H⁺); ¹H NMR (300 MHz, methanol) δ ppm 1.29-1.33 (m, 9H) 1.42 (d, J=9.0 Hz, 3H) 1.94 (s, 3H) 4.53 (s, 2H) 4.83-4.93 (m, 1H) 5.49 (s, 1H) 7.12 (d, J=8.82 Hz, 2H) 7.30-7.37 (m, 2H) 7.48 (t, J=9.38 Hz, 4H).

### EXAMPLE 62

### N-{3-[2-(4-benzoylphenoxy)-1,3-thiazol-5-yl]-1-methylprop-2-ynyl}acetamide

### EXAMPLE 62A

### [4-(5-Bromo-thiazol-2-yloxy)-phenyl]-phenyl-methanone

Example 62A was prepared according to the procedure described in Example 16A substituting 4-hydroxybenzophenone for 4-hydroxybenzaldehyde. The crude product was purified by flash chromatography on silica gel eluting with 5% ethyl acetate/hexanes to provide Example 62a as a white solid (68%). ¹H NMR (300 MHz, DMSO-D6) δ ppm 7.51-7.59 (m, 4H) 7.61 (s, 1H) 7.70 (t, J=7.35 Hz, 1H) 7.73-7.80 (m, 2H) 7.86 (d, J=8.82 Hz, 2H); MS (ESI) m/z 361.7.0 (M+H)⁺.

### EXAMPLE 62B

Example 62B was prepared according to the procedure described in Example 16B substituting Example 62aA for Example 16A. The crude product was purified by reverse-phase HPLC on an Atlantis C18 column (1.9×10 cm, 5 μm particle size) using a gradient of 5% to 100% acetonitrile:0.1% aqueous TFA to provide Example 62B (37%). ¹H NMR (300 MHz, DMSO-D6) δ ppm 1.35 (d, J=6.99 Hz, 3H) 1.82 (s, 3H) 4.77-4.91 (m, 1H) 7.55 (d, J=8.82 Hz, 2H) 7.59 (d, J=7.72 Hz, 2H) 7.65-7.73 (m, 1H) 7.72 (s, 1H) 7.76 (d, J=6.62 Hz, 2H) 7.86 (d, J=8.82 Hz, 2H) 8.44 (d, J=8.09 Hz, 1H); HRMS (M+H)⁺, m/z, (C₂₂H₁₉N₂O₃S): calcd: 391.1111; found:391.1114.

### EXAMPLE 63

### N-{3-[6-(4-benzylphenoxy)pyridin-3-yl]-1-methylprop-2-ynyl}urea

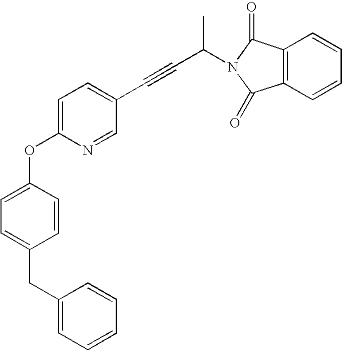

### EXAMPLE 63A

### 2-{3-[6-(4-Benzyl-phenoxy)-pyridin-3-yl]-1-methyl-prop-2-ynyl}-isoindole-1,3-dione

Example 63A was prepared in the exact same manner as Example 10, substituting 2-(1-Methyl-prop-2-ynyl)-isoindole-1,3-dione (CAS [14396-89-5]) for Example 5B. MS (ESI) m/z 460.3 (M+H⁺); ¹H NMR (300 MHz, DMSO-d₆) δ ppm 1.64-1.76 (m, 3H) 3.94 (s, 2H) 5.37 (q, J=6.99 Hz, 1H) 6.95-7.08 (m, 4H) 7.16-7.34 (m, 5H) 7.81-7.94 (m, 6H) 8.18 (d, J=1.84 Hz, 1H).

### EXAMPLE 63b

### 3-[6-(4-Benzyl-phenoxy)-pyridin-3-yl]-1-methyl-prop-2-ynylamine

Example 63A (144 mg, 0.31 mmol) was dissolved in ethanol (5 ml) and hydrazine monohydrate (84.0 μl, 1.7 mmol) was added. The resulting solution was refluxed for 1 h cooled to rt and filtered. The filtrate was concentrated by rotary evaporation and the residue was taken up in CH₂Cl₂ and filtered again to give Example 63B as a solid which was used in the next step without further purification. MS (ESI) m/z 329.1 (M+H⁺).

### EXAMPLE 63

### N-{3-[6-(4-benzylphenoxy)pyridin-3-yl]-1-methylprop-2-ynyl}urea

Example 63B (112 mg, 0.34 mmol) was dissolved in CH₂Cl₂ (2 ml) and cooled to 0° C. Trichloroacetyl isocyanate (56 μl, 0.47 mmol) was added and the solution was stirred for 5 min. The solvent was removed by evaporation and the residue was placed under high vacuum for 1 h. The residue was taken up in MeOH (1 ml), Na₂CO₃ (cat) was added and the reaction mixture was refluxed for 2 h. The solvent was removed by evaporation and the residue was purified by flash column chromatography to provide the title compound, Example 63. MS (ESI) m/z 371.9 (M+H⁺); ¹H NMR (300 MHz, DMSO— d₆) δ ppm 1.34 (d, J=6.99 Hz, 3H) 3.95 (s, 2H) 4.58-4.74 (m, 1H) 5.44-5.62 (m, 1H) 5.76 (s, 2H) 6.45 (d, J=8.46 Hz, 1H) 6.99 (d, J=8.46 Hz, 1H) 7.05 (d, J=8.46 Hz, 2H) 7.24-7.34 (m, 6H) 7.82 (d, J=2.57 Hz, 1H) 7.85 (d, J=2.57 Hz, 1H) 8.16 (d, J=1.84 Hz, 1H).

### EXAMPLE 64

### N-{3-[2-(4-anilinophenoxy)-1,3-thiazol-5-yl]-1-methylprop-2-ynyl}acetamide

The titled compound was prepared according to the procedure described in Example 34, substituting 4-phenylamino-phenol for 3-phenoxy-phenol. ¹H NMR (300 MHz, DMSO-D₆) δ 8.40 (d, J=7.8 Hz, 1H), 8.32 (s, 1H), 7.48 (s, 1H), 7.29-6.83 (m, 9H), 4.85-4.78 (m, 1H), 1.81 (s, 3H), 1.32 (d, J=6.8 Hz, 3H). MS (ESI) positive ion 378 (M+H)⁺; negative ion 376 (M−H)⁻.

### EXAMPLE 65

### N-{1-methyl-3-[6-(3-propylphenoxy)pyridin-3-yl]prop-2-ynyl}acetamide

### EXAMPLE 65A

### 5-Bromo-2-(3-propyl-phenoxy)-pyridine

Example 65A was prepared in the exact same manner as Example 7A, substituting 3-Propyl-phenol for 4-Pentyl-phenol to provide the title compound. MS (ESI) m/z 292.1 (M+H⁺); ¹H NMR (300 MHz, DMSO-d₆) δ ppm 0.89 (t, J=7.35 Hz, 3H) 1.49-1.67 (m, 2H) 2.52-2.60 (m, 2H) 6.90-7.09 (m, 3H) 7.32 (app t, J=7.91 Hz, 1H) 8.03 (d, J=2.94 Hz, 1H) 8.06 (d, J=2.57 Hz, 1H) 8.28 (d, J=2.21 Hz, 1H).

### EXAMPLE 65

### N-{1-methyl-3-[6-(3-propylphenoxy)pyridin-3-yl]prop-2-ynyl}acetamide

Example 65 was prepared in the exact same manner as Example 7, substituting Example 65A for Example 7A. MS (ESI) m/z 323.0 (M+H⁺); ¹H NMR (300 MHz, DMSO-d₆) δ ppm 0.89 (t, J=7.35 Hz, 3H) 1.35 (d, J=6.0 Hz, 3H) 1.51-1.66 (m, 2H) 1.83 (s, 3H) 2.52-2.60 (m, 2H) 4.76-4.90 (m, 1H) 6.91-7.10 (m, 3H) 7.32 (t, J=8.09 Hz, 1H) 7.84 (d, J=2.21 Hz, 1H) 7.87 (d, J=2.57 Hz, 1H) 8.20 (d, J=1.47 Hz, 1H) 8.43 (d, J=7.72 Hz, 1H).

### EXAMPLE 66

### N-[1-methyl-3-(2-{4-[(E)-(phenoxyimino)methyl]phenoxy}-1,3-thiazol-5-yl)prop-2-ynyl]acetamide

Example 66 was prepared according to the procedure described in Example 18 substituting O-phenylhydroxylamine hydrochloride for O-benzylhydroxylamine hydrochloride. The crude product was purified by reverse-phase HPLC on an Atlantis C18 column (1.9×10 cm, 5 μm particle size) using a gradient of 5% to 100% acetonitrile:0.1% aqueous TFA to provide Example 66 (33%) as a purple solid. ¹H NMR (300 MHz, DMSO-D6) δ ppm 1.34 (d, J=6.99 Hz, 3H) 1.82 (s, 3H) 4.77-4.91 (m, 1H) 7.07 (t, J=7.35 Hz, 1H) 7.26 (d, J=7.35 Hz, 2H) 7.37 (t, J=7.91 Hz, 2H) 7.51 (d, J=8.82 Hz, 2H) 7.54 (s, 1H) 7.90 (d, J=8.82 Hz, 2H) 8.43 (d, J=8.09 Hz, 1H) 8.75 (s, 1H); MS (ESI) m/z 406.0 (M+H)⁺.

### EXAMPLE 67

### N-(1-methyl -3-{2-[4-(tetrahydro-2H-pyran-2-ylmethoxy)phenoxy]-1,3-thiazol-5-yl}prop-2-ynyl)acetamide

The titled compound was prepared according to the procedure described in Example 11C, substituting (tetrahydro-pyran-2-yl)-methanol for cyclopentyl-methanol. ¹H NMR (300 MHz, DMSO-D₆) δ 8.40 (d, J=8.1 Hz, 1H), 7.47 (s, 1H), 7.29 (d, J=9.2 Hz, 2H), 7.01 (d, J=9.2 Hz, 2H), 4.85-4.75 (m, 1H), 4.27-3.20 (m, 5H), 1.81 (s, 3H), 1.75-1.09 (m, 6H), 1.32 (d, J=7.1 Hz, 3H). MS (ESI) positive ion 401 (M+H)⁺; negative ion 399 (M−H)⁻.

### EXAMPLE 68

### N-(3-{2-[4-(isopropylthio)phenoxy]-1,3-thiazol-5-yl}-1-methylprop-2-ynyl)acetamide

### EXAMPLE 68A

### 4-Isopropylsulfanyl-phenol

A solution of 4-mercaptophenol (250 mg, 1.98 mmol) in acetone (2.5 mL) was treated with K₂CO₃ (290 mg, 2.1 mmol) and 2-iodopropane (316.9 μL, 3.17 mmol). The reaction was stirred at 25° C. for 18 h and filtered. The filtrate was diluted with diethyl ether (60 mL) and washed with 1N NaOH (2×20 mL). The combined aqueous layers were acidified to pH 1 with 6N HCl and the resulting mixture was extracted with ethyl ether (40 mL). The organic layer was washed with brine (15 mL), dried over Na₂SO₄, and concentrated. The crude product was purified by flash chromatography on silica gel eluting with 10% ethyl acetateihexanes to provide 299 mg (90%) Example 68a as a colorless oil. ¹H NMR (300 MHz, CHLOROFORM-D) δ ppm 1.24 (d, J=6.62 Hz, 6H) 3.03-3.30 (m, 1H) 4.83 (s, 1H) 6.78 (d, J=8.82 Hz, 2H) 7.35 (d, J=8.82 Hz, 2H); MS (DCI) m/z 169.0 (M+H)⁺.

### EXAMPLE 68B

### 5-Bromo-2-(4-isopropylsulfanyl-phenoxy)-thiazole

Example 68B was prepared according to the procedure described in Example 16A substituting Example 68A for 4-hydroxybenzaldehyde. The crude product was purified by flash chromatography on silica gel eluting with 1% ethyl acetate/hexanes to provide Example 68B as a colorless oil (76%). ¹H NMR (300 MHz, DMSO-D6) δ ppm 1.24 (d, J=6.62 Hz, 6H) 3.37-3.65 (m, 1H) 7.35 (d, J=8.82 Hz, 2H) 7.47 (d, J=8.82 Hz, 2H) 7.46 (s, 1H); MS (DCI) m/z 331.9 (M+H)⁺.

### EXAMPLE 68C

Example 68C was prepared according to the procedure described in Example 16B substituting Example 68B for Example 16A. The crude product was purified by flash chromatography on silica gel eluting with 1% MeOH/CHCl₃ to provide Example 68b (55%). ¹H NMR (300 MHz, DMSO-D6) δ ppm 1.24 (d, J=6.62 Hz, 6H) 1.33 (d, J=6.99 Hz, 3H) 1.81 (s, 3H) 3.42-3.57 (m, 1H) 4.71-4.91 (m, 1H) 7.35 (d, J=8.82 Hz, 2H) 7.47 (d, J=8.82 Hz, 2H) 7.50 (s, 1H) 8.42 (d, J=7.72 Hz, 1H); MS (ESI) m/z 361.1 (M+H)⁺.

### EXAMPLE 69

### N-{3-[2-(4-{[isopropyl(methyl)amino]sulfonyl}phenoxy)-1,3-thiazol-5-yl]-1-methylprop-2-ynyl}acetamide

### EXAMPLE 69A

### 4-(5-bromo-thiazol-2-yloxy)-benzenesulfonic acid

4-Hydroxybenzenesulfonic acid (0.543 g, 2.03 mmoL, 65% w/w in H₂O), 2,5-dibromothiazole (0.443 g, 1.82 mmoL), and potassium carbonate (0.730 g, 5.29 mmoL) were dissolved in DMF (2 mL). The mixture was then heated in the microwave at 160° C. for 10 min. The reaction was cooled and the reaction filtered. The filtrate was acidified with 4N HCl in dioxane (2 mL) and filtered again. The filtrate was concentrated in vacuo to provide the title compound (0.453 g, 74%). MS (ESI) m/e 335.7 (M+H); ¹H NMR (300 MHz, DMSO-d₆) δ ppm 7.45 (s, 1H) 7.40 (d, J=8.46 Hz, 2H) 6.66 (d, J=8.46 Hz, 2H).

### EXAMPLE 69B

### 4-(5-bromo-thiazol-2-yloxy)-N-isopropyl-N-methyl-benzenesulfonamide

4-(5-bromo-thiazol-2-yloxy)-benzenesulfonic acid (0.770 g, 2.29 mmoL) and phosphorus pentachloride (0.700 g, 3.37 mmoL) were added to CH₂Cl₂ (10 mL). The mixture was then heated to reflux for 2 hr. Next pyridine (1 mL) was added and heating was continued for 20 hr. More PCl₅ (0.180 g, 0.865 mmoL) and heating was continued for 5 hr. Excess N-methylisopropylamine and excess triethylamine were then added and stirred together at room temperature for 3 hr. The reaction was then acidified with 1N HCl (pH=1). The reaction was extracted with EtOAc (3×25 mL). The combined extracts were washed with H₂O (1×25 mL) followed by brine (1×25 mL). The extracts were dried (Na₂SO₄), filtered, and the solvent removed in vacuo. The crude product was purified by flash chromatography on SiO₂ eluting with a solvent gradient from 10% to 30% EtOAc in hexanes to afford the title compound (0.120 g, 13%). MS (CI) m/e 392.9 (M+H); ¹H NMR (300 MHz, CDCl₃) δ ppm 7.85 (d, 2H) 7.41 (d, 2H) 7.22 (s, 1H) 4.17-4.32 (m, 1H) 2.73 (s, 3H) 1.02 (d, J=6.62 Hz, 6H).

### EXAMPLE 69

### N-{3-[2-(4-{[isopropyl(methyl)amino]sulfonyl}phenoxy)-1,3-thiazol-5-yl]-1-methylprop-2ynyl}acetamide

4-(5-bromo-thiazol-2-yloxy)-N-isopropyl-N-methyl-benzenesulfonamide (0.105 g, 0.269 mmoL), N-(1-methyl-prop-2-ynyl)acetamide (0.040 g, 0.360 mmoL), and triethylamine (2.5 mL) were dissolved in anhydrous THF (5 mL). The solution was degassed then it was treated with dichlorobis(triphenylphosphine)palladium (II) (0.22 g, 0.031 mmoL) followed by copper iodide (0.003 g, 0.016 mmoL). It was then heated to 80° C. for 2 hr. More N-(1-methyl-prop-2-ynyl)acetamide (0.030 g, 0.270 mmoL) and dichlorobis(triphenylphosphine)palladium (II) (0.005 g, 0.007 mmoL) were added and heating was continued for an additional 1 hr. The solvent was removed in vacuo. The crude product was purified by flash chromatography on SiO₂ eluting with a solvent gradient from 30% to 80% EtOAc in hexanes to afford the title compound (0.035 g, 31%). MS (CI) m/e 422.1 (M+H); ¹H NMR (300 MHz, CDCl₃) δ ppm 7.86 (d, 2H) 7.42 (d, 2H) 7.33 (s, 1H) 5.65 (d, 1H) 4.98-5.15 (m, 1H) 4.17-4.32(m, 1H) 2.68-2.77 (m, 3H) 1.99-2.04 (m, 3H) 1.48 (d, J=6.62 Hz, 3H) 1.02 (d, J=6.62 Hz, 6H).

### EXAMPLE 70

### N-{1-methyl-3-[2-(4-phenoxy)phenoxy)pyrimidin-5-yl]prop-2-ynyl}acetamide

The desired product was prepared by substituting 5-bromo-2-chloropyrimidine for 5-bromo-2-fluoropyridine, and 4-phenoxyphenol for Example 2A in Example 32. MS (DCI): m/z 374 (M+H); ¹H NMR (300 MHz, CHLOROFORM-D) δ ppm 1.50 (d, J=6.99 Hz, 3H), 2.02 (s, 3H), 5.00-5.15 (m, 1H), 5.62-5.77 (m, 1H), 7.00-7.09 (m, 4H), 7.10-7.21 (m, 3H), 7.29-7.41 (m, 2H), 8.57 (s, 2H).

### EXAMPLE 71

### N-(3-{2-[4-(1-hydroxy-2-methylpropyl)phenoxy]-1,3-thiazol-5-yl}-1-methylprop-2-ynyl)acetamide

Example 71 was prepared according to the procedures described in Example 16 substituting isopropylmagnesium bromide for isobutylmagnesium bromide in Example 16c. The crude product was purified by reverse-phase HPLC on an Atlantis C18 column (1.9×10 cm, 5 μm particle size) using a gradient of 5% to 100% acetonitrile:0.1% aqueous TFA to provide Example 71 (18%) as an amorphous white solid. ¹H NMR (500 MHz, DMSO-D6) δ ppm 0.76 (d, J=6.71 Hz, 3H) 0.85 (d, J=6.71 Hz, 3H) 1.33 (d, J=7.32 Hz, 3H) 1.71-1.85 (m, 1H) 1.81 (s, 3H) 4.29 (d, J=6.10 Hz, 1H) 4.67-4.92 (m, 1H) 7.30 (d, J=8.54 Hz, 2H) 7.38 (d, J=8.54 Hz, 2H) 7.48 (s, 1H) 8.38 (d, J=7.93 Hz, 1H); MS (ESI) m/z 359.0 (M+H)⁺.

### EXAMPLE 72

### N-{1-methyl-3-[2-(4-phenoxyphenoxy)-1,3-thiazol-5-yl]prop-2-ynyl}acetamide

The titled compound was prepared according to the procedure described in Example 34, substituting 4-phenoxy-phenol for 3-phenoxy-phenol. ¹H NMR (300 MHz, DMSO-D₆) δ 8.41 (d, J=7.8 Hz, 1H), 7.50 (s, 1H), 7.45-7.04 (m, 9H), 4.87-4.78 (m, 1H), 1.82 (s, 3H), 1.33 (d, J=6.8 Hz, 3H). MS (ESI) positive ion 379 (M+H)⁺; negative ion 377 (M−H)⁻.

### EXAMPLE 73

### N-{3-[2-(4-benzylphenoxy)-1,3-thiazol-5-yl]-1-methylprop-2-ynyl}acetamide

### EXAMPLE 73A

### 2-(4-Benzyl-phenoxy)-5-bromo-thiazole

Example 73A was prepared in the exactly same manner as Example 14A, substituting 4-Benzyl-phenol for 4-Pentyl-phenol to provide the title compound. MS (ESI) m/z 346.4 (M+H⁺); ¹H NMR (300 MHz, DMSO-d₆) δ ppm 3.97 (s, 2H) 7.19-7.36 (m, 9H) 7.42 (s, 1H).

### EXAMPLE 73

### N-{3-[2-(4-benzylphenoxy)-1,3-thiazol-5-yl]-1-methylprop-2-ynyl}acetamide

Example 73 was prepared in the exact same manner as Example 73, substituting Example 73A for Example 14A to provide the title compound. MS (ESI) m/z 377.1 (M+H⁺); ¹H NMR (500 MHz, DMSO-d₆) δ ppm 1.32 (d, J=7.32 Hz, 3H) 1.80 (s, 3H) 3.97 (s, 2H) 4.73-4.88 (m, 1H) 7.14-7.38 (m, 9H) 7.45 (s, 1H) 8.37 (d, J=7.32 Hz, 1H)

### EXAMPLE 74

### 3-({5-[3-(acetylamino)but-1-ynyl]-1,3-thiazol-2-yl}oxy)-N-cyclohexylbenzamide

Example 74 was prepared according to the procedures described in Example 44 substituting cyclohexylamine for cyclohexanemethylamine in Example 44c. The crude product was purified by reverse-phase HPLC on an Atlantis C18 column (1.9×10 cm, 5 μm particle size) using a gradient of 5% to 95% acetonitrile:0.1% aqueous TFA to provide 25 mg (38%) of Example 74 as awhite solid. ¹H NMR (300 MHz, DMSO-D6) δ ppm 1.03-1.32 (m, 7H) 1.34 (d, J=6.99 Hz, 3H) 1.55-1.65 (m, 1H) 1.67-1.78 (m, 2H) 1.82 (s, 3H) 3.68-3.84 (m, 1H) 4.74-4.90 (m, 1H) 7.46-7.64 (m, 3H) 7.78-7.88 (m, 2H) 8.30 (d, J=8.09 Hz, 1H) 8.43 (d, J=8.09 Hz, 1H); MS (ESI) m/z 412.0 (M+H)⁺.

### EXAMPLE 75

### N-{1-methyl-3-[2-(4-phenoxyphenoxy)-1,3-thiazol-5-yl]prop-2-ynyl}urea

### EXAMPLE 75A

### 4-[2-(4-phenoxy)phenoxy)-1,3-thiazol-5-yl]but-3-yn-2-ol

The titled compound was prepared according to the procedure described in Example 72, substituting but-3-yn-2-ol for N-(1-methyl-prop-2-ynyl)-acetamide.

### EXAMPLE 75B

### 2-{1-methyl-3-[2-(4-phenoxyphenoxy)-1,3-thiazol-5-yl]prop-2-ynyl}-1H-isoindole-1,3(2H)-dione

To a mixture of Example 75A (2.01 g, 5.96 mmol), phthalimide (1.14 g, 7.75 mmol), and Ph₃P (2.19 g, 8.34 mmol) in THF (16mL) was added DEAD (1.31 mL, 8.34 mmol). The reaction mixture was stirred at room temperature overnight. It was concentrated and purified by flash chromatography on silica gel to get 1.5 g titled compound (yield 54%).

### EXAMPLE 75C

### 1-methyl-3-[2-(4-phenoxyphenoxy)-1,3-thiazol-5-yl]prop-2-ynylamine

The mixture of Example 75B(1.5 g, 3.2 mmol), and hydrazine monohydrate (625 μL, 12.9 mmol) in THF (10 mL) was heated to reflux for 2 hours. It was cooled down to room temperature and filtered off the solid. The filtrate was concentrated and purified by flash chromatography on silica gel with ethyl acetate/methanol (10/1) to get 0.55 g titled compound (yield 51%).

### EXAMPLE 75D

### N-{1-methyl-3-[2-(4-phenoxyphenoxy)-1,3-thiazol-5-yl]prop-2-ynyl}urea

To a mixture of Example 75C (17 mg, 0.05 mmol) in acetic acid (250 μL) was added KOCN (10 mg, 0.11 mmol) in water (250 μL). The reaction mixture was heated at 60° C. overnight. It was partitioned between ethyl acetate and saturated NaHCO₃ (30 mL, 1:1). The organic phase was washed with brine, dried (MgSO₄), filtered and concentrated under reduced pressure. The residue was purified by HPLC to provide 10 mg titled compound (yield 53%). ¹H NMR (300 MHz, DMSO-D₆) δ 7.52-7.03 (m, 10H), 6.45 (d, J=8.5 Hz, 1H), 5.53 (s, 2H), 4.70-4.58 (m, 1H), 1.32 (d, J=7.1 Hz, 3H). MS (ESI) positive ion 380 (M+H)⁺; negative ion 378 (M−H)⁻.

### EXAMPLE 76

### N-{3-[2-(4-cyclohexylphenoxy)-1,3-thiazol-5-yl]-1-methylprop-2-ynyl}acetamide

### EXAMPLE 76A

### 5-Bromo-2-(4-cyclohexyl-phenoxy)-thiazole

Example 76A was prepared in the exact same manner as Example 14A, substituting 4-Cyclohexyl-phenol for 4-Pentyl-phenol to provide the title compound which was used without further purification. MS (ESI) m/z 338.5 (M+H⁺).

### EXAMPLE 76

### N-{3-[2-(4-cyclohexylphenoxy)-1,3-thiazol-5-yl]-1-methylprop-2-ynyl}acetamide

Example 76 was prepared in the exact same manner as Example 14, substituting Example 76a for Example 14a to provide the title compound. MS (ESI) m/z 369.6

(M+H⁺); ¹H NMR (300 MHz, DMSO-d₆) δ ppm 1.21-1.50 (m, 8H) 1.63-1.88 (m, 8H) 2.51-2.61 (m, 1H) 4.69-4.91 (m, 1H) 7.20-7.39 (m, 4H) 7.42-7.53 (m, 1H) 8.41 (d, J=8.09 Hz, 1H).

### EXAMPLE 78

### N-[1-methyl-3-(2-{4-[(E)-(phenylhydrazono)methyl]phenoxy}-1,3-thiazol-5-yl)prop-2-ynyl]acetamide

Example 78 was prepared according to the procedure described in Example 18 substituting phenylhydrazine for O-benzylhydroxylamine hydrochloride. The crude product was purified by reverse-phase HPLC on an Atlantis C18 column (1.9×10 cm, 5 μm particle size) using a gradient of 5% to 100% acetonitrile:0.1% aqueous TFA to provide Example 78 (49%) as a tan solid. ¹H NMR (300 MHz, DMSO-D6) δ ppm 1.33 (d, J=6.99 Hz, 3H) 1.82 (s, 3H) 4.69-4.99 (m, 1H) 6.76 (t, J=7.17 Hz, 1H) 7.07 (d, J=7.35 Hz, 2H) 7.22 (t, J=7.91 Hz, 2H) 7.39 (d, J=8.82 Hz, 2H) 7.52 (s, 1H) 7.74 (d, J=8.82 Hz, 2H) 7.88 (s, 1H) 8.42 (d, J=7.72 Hz, 1H) 10.42 (s, 1H); MS (ESI) m/z 405.0 (M+H)⁺.

### EXAMPLE 79

### N-[3-(2-{4-[(E)-(benzylhydrazono)methyl]phenoxy}-1,3-thiazol-5-yl)-1-methylprop-2-ynyl]acetamide

Example 79 was prepared according to the procedure described in Example 18 substituting benzylhydrazine dihydrochloride for O-benzylhydroxylamine hydrochloride. The crude product was purified by reverse-phase HPLC on an Atlantis C18 column (1.9×10 cm, 5 μm particle size) using a gradient of 5% to 100% acetonitrile:0.1% aqueous TFA to provide Example 79 (26%) as a tan solid. ¹H NMR (300 MHz, DMSO-D6) δ ppm 1.33 (d, J=6.99 Hz, 3 H) 1.81 (s, 3H) 4.35 (s, 2H) 4.71-4.93 (m, 1H) 7.19-7.40 (m, 6H) 7.50 (s, 1H) 7.55 (d, J=8.46 Hz, 2H) 7.61 (s, 1H) 8.41 (d, J=8.09 Hz, 1H); MS (ESI) m/z 419.0 (M+H)⁺.

### EXAMPLE 80

### 3-({5-[3-(acetylamino)but-1-ynyl]-1,3-thiazol-2-yl}oxy)-N-phenylbenzamide

Example 80 was prepared according to the procedures described in Example 44 substituting aniline for cyclohexanemethylamine in Example 44c. The crude product was purified by reverse-phase HPLC on an Atlantis C18 column (1.9×10 cm, 5 μm particle size) using a gradient of 5% to 95% acetonitrile:0.1% aqueous TFA to provide 21 mg (32%) of Example 80 as a white solid. ¹H NMR (300 MHz, DMSO-D6) δ ppm 1.34 (d, J=6.99 Hz, 3H) 1.82 (s, 3H) 4.70-4.96 (m, 1H) 7.12 (t, J=7.35 Hz, 1H) 7.36 (t, J=7.91 Hz, 2H) 7.53 (s, 1H) 7.57-7.71 (m, 2H) 7.76 (d, J=7.72 Hz, 2H) 7.87-8.04 (m, 2H) 8.43 (d, J=8.09 Hz, 1H) 10.32 (s, 1H); MS (ESI) m/z 406.1 (M+H)⁺.

### EXAMPLE 81

### N-(3-{2-[4-(allyloxy)phenoxy]-1,3-thiazol-5-yl}-1-methylprop-2-ynyl)acetamide

### EXAMPLE 81A

### 4-(5-Bromo-thiazol-2-yloxy)-phenol

A solution of 2,5-dibromothiazole (3 g, 12.35 mmol), and hydroquinone (2.04 g, 18.53 mmol) in DMF (30mL) was treated with potassium carbonate (1.71 g, 12.35 mmol) and the mixture was heated at 130° C. for 3 h. The reaction was cooled to 25° C., poured into water (200 mL) and extracted with diethyl ether (3×150 mL). The combined organic layers were washed with water (3×150 mL) and brine (150 mL), dried over sodium sulfate and concentrated on a rotary evaporator. The crude concentrate (3.2 g) was purified by flash chromatography on silica gel eluting with a solvent gradient from 1% to 5% methanol in chloroform to provide 2.1 g (63%) of Example 81 a as a tan solid. ¹H NMR (300 MHz, DMSO-D6) δ ppm 6.82 (d, J=8.82 Hz, 2H) 7.19 (d, J=8.82 Hz, 2H) 7.40 (s, 1H) 9.70 (s, 1H); MS (ESI) m/z 273.7 (M+H)⁺.

### EXAMPLE 81B

### N-{3-[2-(4-Hydroxy-phenoxy)-thiazol-5-yl]-1-methyl-prop-2-ynyl}-acetamide

A solution of Example 81a (201 mg, 0.74 mmol), Example 5b (97 mg, 0.88 mmol) and Et₃N (358 μL, 2.57 mmol) in THF (3.5 mL) was degassed and treated with Pd(PPh₃)₂Cl₂ (20.6 mg, 0.03 mmol) and CuI (2.8 mg, 0.015 mmol). The reaction was heated under nitrogen at 75° C. for 2.5 h. The solvent was evaporated and the crude concentrate was purified by flash chromatography on silica gel eluting with a solvent gradient from 1% to 5% methanol in chloroform to provide 200 mg (90%) of Example 81b as a tan solid. ¹H NMR (300 MHz, DMSO-D6) δ ppm 1.31 (d, J=6.99 Hz, 3H) 1.81 (s, 3H) 4.66-4.95 (m, 1H) 6.82 (d, J=8.82 Hz, 2H) 7.19 (d, J=8.82 Hz, 2H) 7.47 (s, 1H) 8.40 (d, J=7.72 Hz, 1H) 9.69 (s, 1H).

### EXAMPLE 81C

### N-(3-{2-[4-(allyloxy)phenoxy]-1,3-thiazol-5-yl}-1-methylprop-2-ynyl)acetamide

A 0° C. mixture of Example 81B (30 mg, 0.099 mmol) and potassium carbonate (13.7 mg, 0.099 mmol) in DMF (0.5 mL) was treated with allyl bromide (34.6 μL, 0.4 mmol). The resulting mixture was stirred for 10 min at 0° and for 5 h at 25° C. The reaction was filtered and the filtrate concentrated on a rotary evaporator. The crude concentrate was purified by reverse-phase HPLC on an Atlantis C18 column (1.9×10 cm, 5 μm particle size) using a gradient of 5% to 100% acetonitrile:0.1% aqueous TFA to provide 14 mg (41%) of Example 81C. ¹H NMR (300 MHz, DMSO-D6) δ ppm 1.32 (d, J=6.99 Hz, 3H) 1.81 (s, 3H) 4.59 (d, J=5.15 Hz, 2H) 4.72-4.91 (m, 1H) 5.28 (dd, J=10.48, 1.65 Hz, 1H) 5.41 (dd, J=17.28, 1.84 Hz, 1 H) 5.87-6.19 (m, 1H) 7.04 (d, J=9.19 Hz, 2H) 7.32 (d, J=8.82 Hz, 2H) 7.48 (s, 1 H) 8.40 (d, J=7.72 Hz, 1H); HRMS (M+H)⁺, m/z, (C₁₈H₁₉N₂O₃S): calcd:343.1111; found:343.1110.

### EXAMPLE 82

### N-(3-{2-[4-(benzyloxy)phenoxy]-1,3-thiazol-5-yl}-1-methylprop-2-ynyl)acetamide

A Smith Process vial (0.5-2 ml) was charged with a stir bar. To the vessel were added dibromothiazole(36 mg, 0.15 mmol) and benzyl alcohol (0.16 mmol). MP-Carbonate (0.45 mmol, 3 mmol/g loading) was added to above solution. The reaction vessel was sealed and heated in microwave to 150° C. for 1200 s. After cooling, the reaction vessel was uncapped. Without further work-up, triethylamine (63 μl, 0.45 mmol ) and Example 5B (18 mg, 0.16 mmol) were added to the resulting mixture followed by PdCl₂(PPh₃)₂ (5.3 mg, 0.0075 mmol) and CuI (1 mg, 0.0045 mmol). The resulting reaction mixture was capped and heated in microwave to 150° C. for another 1200 s. After cooling, the vessel was again uncapped and filtered through a small plug of silica gel. The filtrate was collected and dried. HPLC purification gave the desired products. ¹H NMR δ, 1.32 (d, 3H, J=6.8 Hz), 1.81 (s, 3H), 4.81 (m, 1H), 5.12 (s, 2H), 7.10 (d, 2H, J=9.4 Hz), 7.32 (d, 2H, J=9.4 Hz), 7.39-7.46 (m, 6H), 8.37 (d, 1H, 7.8 Hz); MS (ESI) m/z 393.2 (M+H).

### EXAMPLE 83

### 3-({5-[3-(acetylamino)but-1-ynyl]-1,3-thiazol-2-yl}oxy)-N-isobutylbenzamide

Example 83 was prepared according to the procedures described in Example 44 substituting iosbutylamine for cyclohexanemethylamine in Example 44c. The crude product was purified by reverse-phase HPLC on an Atlantis C18 column (1.9×10 cm, 5 μm particle size) using a gradient of 5% to 95% acetonitrile:0.1% aqueous TFA to provide 20 mg (32%) of Example 83 as a white solid. ¹H NMR (300 MHz, DMSO-D6) δ ppm 0.88 (d, J=6.62 Hz, 6H) 1.34 (d, J=6.99 Hz, 3H) 1.74-1.93 (m, 4H) 3.00-3.15 (m, 2H) 4.74-4.91 (m, 1H) 7.46-7.66 (m, 3H) 7.75-7.88 (m, 2H) 8.43 (d, J=7.72 Hz, 1H) 8.56 (t, J=5.70 Hz, 1H); MS (ESI) m/z 386.0 (M+H)⁺.

### EXAMPLE 84

### N-{3-[6-(4-{[isopropyl(methyl)amino]sulfonyl}phenoxy)pyridin-3-yl]-1-methylprop-2-ynyl}acetamide

### EXAMPLE 84A

### 4-(5-bromo-pyridin-2-yloxy)-benzenesulfonic acid

Example 84A was prepared according to the procedure described in Example 69A substituting 5-Bromo-2-fluoropyridine for 2,5-dibromothiazole. The title compound (1.91 g) was carried on to the next step without purification.

### EXAMPLE 84B

### 4-(5-bromo-pyridin-2-yloxy)-N-isopropyl-N-methyl-benzenesulfonamide

Example 84b was prepared according to the procedure described in Example 69b substituting 4-(5-bromo-pyridin-2-yloxy)-benzenesulfonic acid for 4-(5-bromo-thiazol-2-yloxy)-benzenesulfonic acid. The crude product was purified by flash chromatography on SiO₂ eluting with a solvent gradient from 30% to 80% EtOAc in hexanes to afford the title compound (0.073 g, 69%). MS (CI) m/e 386.9 (M+H); ¹H NMR (300 MHz, CDCl₃) δ ppm 8.25 (d, J=2.57 Hz, 1H) 7.79-7.92 (m, 2H) 7.19-7.29 (m, 2H) 6.88-6.95 (m, J=8.09 Hz, 1H) 2.70-2.78 (m, 3H) 1.03 (d, J=6.62 Hz, 6H).

### EXAMPLE 84

### N-{3-[6-(4-{[isopropyl(methyl)amino]sulfonyl}phenoxy)pyridin-3-yl]-1-methylprop-2-ynyl}acetamide

Example 84B was prepared according to the procedure described in Example 69 substituting 4-(5-bromo-pyridin-2-yloxy)-N-isopropyl-N-methyl-benzenesulfonamide for 4-(5-bromo-thiazol-2-yloxy)-N-isopropyl-N-methyl-benzenesulfonamide. The crude product was purified by flash chromatography on SiO₂ eluting with a solvent gradient from 30% to 80% EtOAc in hexanes to afford the title compound (0.035 g, 31%). MS (CI) m/e 416.1 (M+H); ¹H NMR (300 MHz, CDCl₃) δ ppm 8.24 (d, J=1.84 Hz, 1H) 7.83 (d, J=8.82 Hz, 2H) 7.75 (dd, J=8.46, 2.21 Hz, 1H) 7.24 (d, J=8.82 Hz, 2H) 6.92 (d, J=8.46 Hz, 1H) 5.69 (d, J=7.35 Hz, 1H) 4.99-5.14 (m, 1H) 4.19-4.33 (m, 1H) 2.73 (s, 3H) 2.02 (s, 3H) 1.50 (d, J=6.99 Hz, 3H) 1.03 (d, J=6.62 Hz, 6H).

### EXAMPLE 85

### N-(3-{2-[4-(isobutylamino)-3-methylphenoxy]-1,3-thiazol-5-yl}-1-methylprop-2-ynyl)acetamide

The titled compound was prepared according to the procedure described in Example 34, substituting 4-isobutylamino-3-methyl-phenol for 3-phenoxy-phenol. ¹H NMR (300 MHz, DMSO-D₆) δ 8.38 (d, J=8.1 Hz, 1H), 7.45 (s, 1H), 7.03-6.97 (m, 2H), 6.55-6.50 (m, 1H), 5.00 (t, J=6.1 Hz, 1H), 4.85-4.75 (m, 1H), 2.90 (d, J=6.1 Hz, 2H), 2.10 (s, 3H), 1.97-1.85 (m, 1H), 1.80 (s, 3H), 1.31 (d, J=7.1 Hz, 3H), 0.93 (d, J=6.4 Hz, 6H), MS (ESI) positive ion 372 (M+H)⁺; negative ion 370(M−H)⁻.

### EXAMPLE 86

### N-{3-[2-(4-isopropoxyphenoxy)pyrimidin-5-yl]-1-methylprop-2-ynyl}acetamide

The desired product was prepared by substituting 5-bromo-2-chloropyrimidine for 5-bromo-2-fluoropyridine in Example 32. MS (DCI): m/z 340 (M+H); ¹H NMR (300 MHz, CHLOROFORM-D) δ ppm 1.35 (d, J=6.25 Hz, 6H), 1.50 (d, J=6.62 Hz, 3H), 2.02 (s, 3 H), 4.39-4.62 (m, 1H), 5.66 (s, 1H), 6.84-6.99 (m, 2H), 7.02-7.16 (m, 2H), 8.55 (s, 2H).

### EXAMPLE 87

### N-(3-{4-[(4-isopropoxyphenyl)amino]phenyl}-1-methylprop-2-ynyl)acetamide

### EXAMPLE 87A

### (4-Bromo-phenyl)-(4-isopropoxy-phenyl)-amine

A mixture of 3-isopropoxyaniline (900 mg, 5.95 mmol), 4-bromophenyl iodide (1.423 mg, 5.03 mmol), K₃PO₄ (2.120 mg, 9.99 mmol), ethylene glycol (560 μL, 10.04 mmol), and isoproanol (5 mL) was heated to 120° C. under MW for 30 min. The mixture was poured into water, extracted with ether (1×). The ether layer was washed with brine (1×), dried over MgSO₄ and concentrated. The residue was purified on silica gel eluting with ethyl acetate and hexane to give the desired product as a yellow solid (304.5 mg, 16.7%).

### EXAMPLE 87B

### N-(3-{4-[(4-isopropoxyphenyl)amino]phenyl}-1-methylprop-2-ynyl)acetamide

The desired product was prepared by substituting Example 87A for Example 2B in Example 2C. MS (DCI): m/z 337 (M+H); ¹H NMR (300 MHz, CHLOROFORM-D) δ ppm 1.34 (d, J=5.88 Hz, 6H), 1.47 (d, J=6.62 Hz, 3H), 2.00 (s, 3H), 4.40-4.58 (m, 1H), 4.94-5.09 (m, 1H), 5.54-5.63 (br. s, 1H), 5.66-5.75 (m, 1H), 6.72-6.82 (m, 2H), 6.84-6.87 (m, 2H), 6.97-7.11 (m, 2H), 7.19-7.28 (m, 2H).

### EXAMPLE 88

### N-[1-methyl-3-(2-{3-[4-(trifluoromethyl)phenoxy]phenoxy}-1,3-thiazol-5-yl)prop-2-ynyl]acetamid

### EXAMPLE 88A

### 5-bromo-2-{4-[4-(trifluoromethyl)phenoxy]phenoxy}-1,3-thiazole

A solution of 2,5-dibromothiazole (243 mg, 1 mmol), and 4-(4-trifluoromethyl-phenoxy)-phenol (255 mg, 1 mmol) in DMF (10 mL) was treated with potassium carbonate (139 mg, 1 mmol) and the mixture was heated at 130° C. for 2 h. The reaction was cooled to 25° C., poured into water (40 mL) and extracted with diethyl ether (3×45 mL). The combined organic layers were washed with water (3×30 mL) and brine (30 mL), dried over sodium sulfate and concentrated on a rotary evaporator. The crude concentrate (3.2 g) was purified by flash chromatography on silica gel eluting with a solvent gradient from 10% to 25% ethyl acetate in hexanes to provide 400 mg (96%) of Example 88A.

### EXAMPLE 88B

### N-[1-methyl-3-(2-{4-[4-(trifluoromethyl)phenoxy]phenoxy}-1,3-thiazol-5-yl)prop-2-ynyl]acetamide

A solution of Example 88A (124 mg, 0.3 mmol), Example 5B (47 mg, 0.42 mmol), and Et₃N (137 μL, 0.9 mmol) in THF (3 mL) was degassed and treated with Pd(PPh₃)₂Cl₂ (1 mg, 0.15 mmol) and CuI (1.5 mg, 0.0075 mmol). The reaction was heated under nitrogen at 80° C. for 2.5 h. The solvent was evaporated and the crude concentrate was purified by flash chromatography on silica gel eluting with a solvent gradient from 65% to 95% ethyl acetate in hexanes to provide 80 mg (60%) of Example 88B as a light yellow solid. ¹H NMR (300 MHz, DMSO-D6) δ ppm 1.34 (d, J=6.99 Hz, 3H) 1.82 (s, 3H) 4.73-4.91 (m, 1H) 7.18 (d, J=8.46 Hz, 2H) 7.22-7.29 (m, 2H) 7.45-7.50 (m, 2H) 7.51 (s, 1H) 7.77 (d, J=8.46 Hz, 2H) 8.42 (d, J=7.72 Hz, 1H); MS (ESI) m/z 447.1 (M+H)⁺.

### EXAMPLE 89

### N-{1-methyl-3-[2-(4-phenoxy)phenoxy)-1,3-thiazol-5-yl]prop-2-ynyl}isoxazol-3-amine

### EXAMPLE 89A

### N-Isoxazol-3-yl-4-nitro-benzenesulfonamide

To a solution of 3-aminoisoxazole (1.7 g, 0.02 mol) in pyridine at 0° C. was added p-nitrobenzenesulfonyl chloride (4.9 g, 0.022 mol) in portions. The reaction mixture was stirred overnight while warming up to room temperature. Water was added and extracted with ethyl acetate, organic layer was washed with 1N citric acid solution, brine, dried over magnesium sulfate and filtered. The filtrate was concentrated to afford 6.58 g of crude as a light yellow solid. This crude was purified by recrystallization from EA/hexane to give 4.9 g of product as a slightly yellow crystal (91% yield). 1H NMR (300 MHz, DMSO-D6) δ ppm 6.47 (d, J=1.84 Hz, 1H), 8.00-8.27 (m, 2H), 8.30-8.55 (m, 2H), 8.78 (d, J=1.84 Hz, 1H), 11.98 (s, 1H). MS (ESI), M/Z:267.8 (M−H)⁺.

### EXAMPLE 89B

### N-Isoxazol-3-yl-N-{1-methyl-3-[2-(4-phenoxy-phenoxy)-thiazol-5-yl]-prop-2-ynyl}-4-nitro-benzenesulfonamide

To a solution of N-isoxazol-3-yl-4-nitro-benzenesulfonamide (Example 89A, 0.05 g, 0.18 mmol), triphenylphosphine (0.073 g, 0.28 mmol), and 4-[2-(4-phenoxy-phenoxy)-thiazol-5-yl]-but-3-yn-2-ol (??, 0.06 g, 0.18 mmol) in dry THF at room temperature was added a solution of DEAD (0.05 g, 0.28 mmol) in THF dropwise. The reaction mixture was stirred overnight. After the removal of solvent, the crude was purified by chromatography on silica gel (EtOAc/hex, 10-40%) to give 0.083 g ofthe product as a yellow oil (78% yield). 1H NMR (300 MHz, CHLOROFORM-D) δ ppm 1.55 (d, J=6.99 Hz, 3H) 5.46 (q, J=6.99 Hz, 1H) 6.62 (d, J=1.84 Hz, 1H) 6.92-7.53 (m, 10H) 7.94-8.13 (m, 2H) 8.20-8.36 (m, 2H) 8.45 (d, J=1.84 Hz, 1H). MS (ESI), M/Z:588.9 (M+H)⁺.

### EXAMPLE 89

### N-{1-methyl-3-[2-(4-phenoxy)phenoxy)-1,3-thiazol-5-yl]prop-2-ynyl}isoxazol-3-amine

To a solution of N-isoxazol-3-yl-N-{1-methyl-3-[2-(4-phenoxy-phenoxy)-thiazol-5-yl]-prop-2-ynyl}-4-nitro-benzenesulfonamide (Example 89B, 0.34 g, 0.58 mmol) in DMF was added potassium carbonate (0.32 g, 2.3 mmol) and thiophenol (0.077 g, 0.7 mmol) at room temperature. The mixture was stirred for 2 h and TLC indicated the completion. The reaction mixture was treated with 1N NaOH solution, extracted with ethyl acetate. Organic layer was washed with brine, dried over magnesium sulfate and filtered. The filtrate was concentrated and purified on silica gel (EA/hex, 10˜40%) to give 0.16 g of the desired product as a light yellow oil (69% yield). MS (ESI), M/Z:499.9 (M+H)⁺.

### EXAMPLE 90

### 4-[2-(3-phenoxyphenoxy)-1,3-thiazol-5-yl]but-3-yn-2-ol

The titled compound was prepared according to the procedure described in Example 34, substituting but-3-yn-2-ol for N-(1-methyl-prop-2-ynyl)-acetamide. ¹H NMR (300 MHz, DMSO-D₆) δ 7.52-6.93 (m, 10H), 5.55 (d, J=5.4 Hz, 1H), 4.62-4.56 (m, 1H), 1.35 (d, J=6.4 Hz, 3H). MS (ESI) 338 (M+H)⁺.

### EXAMPLE 91

### N-(3-{2-[3-(cyclohexylmethoxy)-5-methylphenoxy]-1,3-thiazol-5-yl}-1-methylprop-2-ynyl)acetamide

### EXAMPLE 91A

### 3-[(5-bromo-1,3-thiazol-2-yl)oxy]-5-methylphenol

A solution of 2,5-dibromothiazole (200 mg, 0.82 mmol), and 5-methyl-benzene-1,3-diol (204 mg, 1.64 mmol) in DMF (8 mL) was treated with potassium carbonate (115 g, 0.82 mmol) and the mixture was heated at 130° C. for 2 h. The reaction was cooled to 25° C., poured into water (30 mL) and extracted with diethyl ether (3×35mL). The combined organic layers were washed with water (3×25 mL) and brine (25 mL), dried over sodium sulfate and concentrated on a rotary evaporator. The crude concentrate (350 mg) was purified by flash chromatography on silica gel eluting with a solvent gradient from 10% to 25% ethyl acetate in hexanes to provide 191 mg (81%) of Example 91a as a white solid. ¹H NMR (300 MHz, DMSO-D6) δ ppm 2.24 (s, 3H) 6.01 (s, 1H) 6.41-6.64 (m, 2H) 7.44 (s, 1H) 9.01 (s, 1H); MS (ESI) m/z 283.8 (M+H)⁺.

### EXAMPLE 91B

### N-{3-[2-(3-hydroxy-5-methylphenoxy)-1,3-thiazol-5-yl]-1-methylprop-2-ynyl}acetamide

A solution of Example 91A (129 mg, 0.45 mmol), Example 5B (60 mg, 0.54 mmol), and Et₃N (188 μL, 1.35 mmol) in THF (3 mL) was degassed and treated with Pd(PPh₃)₂Cl₂ (16 mg, 0.023 mmol) and CuI (2.2 mg, 0.0113 mmol). The reaction was heated under nitrogen at 80° C. for 2.5 h. The solvent was evaporated and the crude concentrate was purified by flash chromatography on silica gel eluting with a solvent gradient from 65% to 95% ethyl acetate in hexanes to provide 107 mg (75%) of Example 91b as a light yellow solid. ¹H NMR (300 MHz, methanol) δ ppm 1.41 (d, J=6.99 Hz, 3H) 1.93 (s, 3H) 2.29 (s, 3H) 4.87-4.96 (m, 1H) 6.44-6.52 (m, 1H) 6.52-6.63 (m, J=7.72 Hz, 3H) 7.32 (s, 1H); MS (ESI) m/z 317.0(M+H)⁺.

### EXAMPLE 91C

### N-(3-{2-[3-(cyclohexylmethoxy)-5-methylphenoxy]-1,3-thiazol-5-yl}-1-methylprop-2-ynyl)acetamide

To a solution of Example 91B (87 mg, 0.27 mmol), cyclohexylmethanol (54 uL, 0.44 mmol), and triphenylphosphine (115 mg, 0.44 mmol) in THF (2 mL) was added diethyl azodicarboxylate (70 uL, 0.44 mmol) dropwise over 2 min at ambient temperature under nitrogen. The reaction mixture was stirred for 6 hours and concentrated in vacuo with a rotary evaporator. The crude concentrate was purified by reverse-phase HPLC on an Atlantis C18 column (1.9×10 cm, 5 μm particle size) using a gradient of 5% to 95% acetonitrile:0.1% aqueous TFA to provide 42 mg (37%) of Example 91C. ¹H NMR (300 MHz, DMSO-D6) δ ppm 0.96-1.12 (m, 2H) 1.15-1.29 (m, 3H) 1.33 (d, J=6.99 Hz, 3H) 1.61-1.78 (m, 6H) 1.81 (s, 3H) 2.28 (s, 3H) 3.77 (d, J=5.88 Hz, 2H) 4.71-4.91 (m, 1H) 6.71-6.83 (m, 3H) 7.50 (s, 1H) 8.41 (d, J=7.72 Hz, 1H); MS (ESI) m/z 413.1 (M+H)⁺.

### EXAMPLE 92

### N-(3-{2-[3-(cyclohexylmethoxy)-5-methylphenoxy]-1,3-thiazol-5-yl}-1-methylprop-2-ynyl)acetamide

### EXAMPLE 92A

### 4-Bromo-2-(4-isopropoxy-phenoxy)-thiazole

To a solution of 4-Isopropoxy-phenol (CAS# 7495-774, 1.62 g, 10.64 mmole) in DMSO (25 mL) was added in turn, potassium carbonate (3 g, 21.7 mmole) and 2,5-dibromothiazole (2.64 g, 11.7 mmole). The reaction mixture was heated at 95° C. overnight. The reaction mixture was cooled down, added water and extracted with ethyl acetate (2×40 mL). The reaction mixture was diluted with ethyl acetate (100 mL), added water (100 mL) and separated. The organic phase was washed with water (100 mL×2) and brine, dried over magnesium sulfate and evaporated in vacuum. The product was purified on silica column using a gradient of 3-10% ethyl acetate in hexane and yielded 3.11 g (99%) of white solid. 1H NMR (300 MHz, CHLOROFORM-D) δ ppm 7.07-7.23 (m, 3H) 6.81-6.96 (m, 2H) 4.40-4.62 (heptet, J=6.25 Hz, 1H) 1.34 (d, J=6.25 Hz, 4H), MS (ESI) M/Z 315 (M+1)⁺.

### EXAMPLE 92B

### 2-{3-[2-(4-Isopropoxy-phenoxy)-thiazol-5-yl]-1-methyl-prop-2-ynyl}-isoindole-1,3-dione

To a degassed solution of 5-Bromo-2-(4-isopropoxy-phenoxy)-thiazole (Example 92A, 1.06 g, 3.4 mmole) and 2-(1-Methyl-prop-2-ynyl)-isoindole-1,3-dione (CAS# 14396-89-5, 806 mg, 4.0 mmole) in tetrahydrofuran (40.0 mL) was added dichlorobis(triphenylphosphine)palladium(II) (118 mg, 0.17 mmole) and copper(I) iodide (16 mg, 0.08 mmole) and heated at reflux for 3.5 hours. The solvent was removed in vacuum and the product was isolated via silica column using a gradient of 5 to 25% ethyl acetate in hexane. Yield of the light-cream solid, 1.24 g. 1H NMR (300 MHz, CHLOROFORM-D) δ 7.80-7.93 (m, 2H) 7.66-7.79 (m, 3H) 7.07-7.21 (m, 2H) 6.81-6.97 (m, 2H) 5.40 (q, J=6.25 Hz, 1 H) 4.41-4.60 (heptet, J=6.25 Hz, 1H) 1.75 (d, J=7.35 Hz, 3H) 1.34 (d, J=6.25 Hz, 6H), MS (ESI) M/Z 433 (M+1)⁺.

### EXAMPLE 92C

### 3-[2-(4-Isopropoxy-phenoxy)-thiazol-5-yl]-1-methyl-prop-2-ynylamine

To a solution of 2-{3-[2-(4-Isopropoxy-phenoxy)-thiazol-5-yl]-1-methyl-prop-2-ynyl}-isoindole-1,3-dione (Example 92B, 736 mg, 1.7 mmole) in methylene chloride (10 mL), at room temperature was added hydrazine hydrate (825 uL, 17 mmole). The reaction mixture was added ethanol (5 mL) and heated at reflux. After 15 minutes the solvent was evaporated to dryness; the resulting slurry was treated with methylene chloride and the solid was removed by filtration. The product was purified on silica column, using first methylene chloride followed by ethyl acetate as eluent. Yield if the yellow oil, 382 mg. 1H NMR (300 MHz, CHLOROFORM-D) δ ppm 7.22-7.30 (m, 1H) 7.11-7.22 (m, 2H) 6.84-6.98 (m, 2H) 4.42-4.60 (heptet, J=6.25 Hz, 1H) 3.90 (q, J=6.99 Hz, 1H) 1.41 (d, J=6.99 Hz, 3H) 1.34 (d, J=6.25 Hz, 6H), MS (ESI) M/Z 303 (M+1)⁺.

### EXAMPLE 92

### N-(3-{2-[3-(cyclohexylmethoxy)-5-methylphenoxy]-1,3-thiazol-5-yl}-1-methylprop-2-ynyl)acetamide

To a solution of 3-[2-(4-Isopropoxy-phenoxy)-thiazol-5-yl]-1-methyl-prop-2-ynylamine (107 mg, 0.35 mmole) in methylene chloride (20 mL) was added TEA (500 uL) followed by the methylchloroformate (55 uL, 0.7 mmole). After an hour the reaction mixture was added methanol (1 mL) and stirred for an hour. The solvent was removed in vacuum and the product was purified via silica column, using a gradient of 5-25% ethyl acetate in hexane as eluent. Yield of the white solid 100.0 mg. 1H NMR (300 MHz, CHLOROFORM-D) δ ppm 7.28 (s, 1H) 7.11-7.21 (m, 2H) 6.84-6.96 (m, 2H) 4.65-5.00 (m, J=6.99 Hz, 2H) 4.41-4.61 (m, 1H) 3.69 (s, 3H) 1.46 (d, J=6.62 Hz, 3H) 1.34 (d, J=5.88 Hz, 6H), MS (ESI) M/Z 361 (M+1)⁺.

### EXAMPLE 93

### N-{1-methyl-3-[2-(4-phenoxyphenoxy)-1,3-thiazol-5-yl]prop-2-ynyl}guanidine-2-ynyl)acetamide

To a solution of Example 75C (150 mg, 0.45 mmol) and 2-ethyl-isothiourea HBr salt (500 mg, 2.7 mmol) in methanol (5 mL) was added Et₃N (627 uL, 4.5 mmol). The reaction mixture was heated at 65° C. for 36 h and concentrated in vacuo with a rotary evaporator. The crude product was purified by reverse-phase HPLC on an Atlantis C18 column (1.9×10 cm, 5 μm particle size) using a gradient of 5% to 95% acetonitrile:0.1% aqueous TFA to provide 10 mg (6%) of Example 93. ¹H NMR (300 MHz, DMSO-D6) δ ppm 1.32 (d, J=6.99 Hz, 3H) 4.56-4.72 (m, 1H) 5.54 (s, 2H) 6.45 (d, J=8.46 Hz, 1H) 7.02-7.13 (m, 4H) 7.17 (t, J=7.35 Hz, 1 H) 7.37-7.46 (m, 4H) 7.45 (s, 1H), 7.48 (s, 1H); MS (ESI) m/z 379.9(M+H)⁺.

### EXAMPLE 94

### N-(3-{6-[(4-isopropoxyphenyl)amino]pyridin-3-yl}-1-methylprop-2-ynyl)acetamide

### EXAMPLE 94A

### (5-Bromo-pyridin-2-yl)-(4-isopropoxy-phenyl)-amine

The mixture of 4-isopropoxyaniline (1.277 g, 8.445 mmol) and 2,5-dibromopyridine (1.000 g, 4.221 mmol) heated to 180° C. for 3 h. The mixture was diluted with ether and washed with water (1×), dried over MgSO₄ and concentrated. The residue was purified on silica gel eluting with ethyl acetate gradient to give the desired product as an off white solid (1.1268 g, 86.9%).

### EXAMPLE 94B

### N-(3-{6-[(4-isopropoxyphenyl)amino]pyridin-3-yl}-1-methylprop-2-ynyl)acetamide

The desired product was prepared by substituting Example 94A for Example 2B in Example 2C. MS (DCI): m/z 338 (M+H); ¹H NMR (300 MHz, CHLOROFORM-D) δ ppm 1.34 (d, J=5.88 Hz, 6H), 1.48 (d, J=6.62 Hz, 3H), 2.01 (s, 3H), 4.51 (heptet, J=5.88 Hz, 1H), 4.95-5.13 (m, 1H), 5.69 (s, 1H), 6.49 (s, 1H), 6.60 (d, J=8.46 Hz, 1H), 6.80-6.95 (m, 2H), 7.13-7.24 (m, 2H), 7.44 (dd, J=8.64, 2.02 Hz, 1H).

### EXAMPLE 95

### N-hydroxy-N′-{1-methyl-3-[2-(4-phenoxy)phenoxy)-1,3-thiazol-5-yl]prop-2-ynyl}urea

### EXAMPLE 95A

### 2-(4-Phenoxy-phenoxy)-thiazole

To a solution of 4-phenoxy-phenol (11.11 g) in DMSO (60 mL) was added, in turn, 2-bromo-thiazole (9.8 g, 0.06 mol) and potassium carbonate (8.25 g, 0.06 mol) and heated at 100° C. overnight. The reaction mixture was exrtracted with methylene chloride (180 mL), washed with water (3×100 mL) and brine, dried over magnesium sulfate filtered and evaporated. The product was purified over silica column using a gradient of 3 to 10% ethyl acetate in hexane and yielded 8.9 g of light yellow oil. 1H NMR (300 MHz, CHLOROFORM-D) δ ppm 7.30-7.40 (m, 2H) 7.19-7.30 (m, 3H) 7.08-7.16 (m, 1H) 6.99-7.08 (m, 4H) 6.81 (d, J=4.04 Hz, 1H); MS (ESI) m/z 270 (M+H)⁺.

### EXAMPLE 95B

### 5-Iodo-2-(4-phenoxy-phenoxy)-thiazole

To a solution of 2-(4-phenoxy-phenoxy)-thiazole (2.34 g, 0.087 mol) in tetrahydrofuran (75 mL) at −78° C., was added a solution of 2.5M BuLi in hexane (3.74 mL, 0.087 mol), stirred for two hour and added iodine (2.2 g, 0.087 mol). The reaction mixture was allowed to and warm up to room temperature. Upon reaching room temperature, the reaction mixture was added ethyl acetate (100 mL) and washed with an aqueous solution of 10% NaHSO3. The ethyl acetate solution was washed with water (2×50 mL) and brine, dried over magnesium sulfate filtered and evaporated. The product was purified over silica column using a gradient of 2 to 10% ethyl acetate in hexane. Yield of the light-orange oil, 3.1 g. 1H NMR (300 MHz, CHLOROFORM-D) δ ppm 7.31-7.40 (m, 2H) 7.30 (s, 1H) 7.18-7.25 (m, 2H) 7.09-7.17 (m, 1H) 6.98-7.08 (m, 4H); MS (ESI ) m/z 396 (M+H)⁺.

### EXAMPLE 95C

### 5-[3-(tert-Butyl-dimethyl-silanyloxy)-but-1-ylyl]-2-(4-phenoxy-phenoxy)-thiazole

To a degassed solution of 5-iodo-2-(4-phenoxy-phenoxy)-thiazole (Example 95B, 1.15 g, 0.0029 mol), tert-butyl-dimethyl-(1-methyl-prop-2-ynyloxy)-silane (CAS 125494-93-1, 1.07 g, 0.0058 mol) and triethyl amine (2.05 mL, 0.015 mol) in acetonitrile (35 mL) at room temperature, was added dichlorobis(triphenylphosphine)palladium(II) (102 mg, 0.00015 mol) and copper(I) iodide (14 mg, 0.000075 mol). After 15 minutes the solvent was removed in vacuum and the reaction mixture was separated over silica column using a gradient of 3 to 10% ethyl acetate in hexane to give 1.23 g of the light reddish solid. 1H NMR (300 MHz, CHLOROFORM-D) δ ppm 7.31-7.41 (m, 2H) 7.19-7.31 (m, 4H) 7.09-7.19 (m, 1H) 6.99-7.09 (m, 3H) 4.71 (q, J=6.62 Hz, 1H) 1.47 (d, J=6.62 Hz, 3H) 0.91 (s, 9H) 0.14 (s, 3H) 0.13 (s, 3H); MS (ESI) m/z 452(M+H)⁺.

### EXAMPLE 95D

### 4-[2-(4-Phenoxy-phenoxy)-thiazol-5-yl]-but-3-yn-2-ol

To a solution of 5-[3-(tert-butyl-dimethyl-silanyloxy)-but-1-ynyl]-2-(4-phenoxy-phenoxy)-thiazole (Example 95C, 1.2 g, 2.65 mmole) in tetrahydrofuran (5 mL), at room temperature, was added a solution of 1N tetrabutylamonnium fluoride (4 mL, 4.0 mmole) in tetrahydrofuran (4 mL) and stirred for 10 minutes. The reaction mixture was poured over silica and washed with ethyl acetate to yield the product as white solid in quantitative yield (896 mg). 1H NMR (300 MHz, CHLOROFORM-D) δ ppm 7.32-7.40 (m, 2H) 7.31 (s, 1H) 7.20-7.26 (m, 2H) 7.09-7.18 (m, J=7.35, 7.35 Hz, 1H) 6.98-7.09 (m, 4H) 4.65-4.81 (m, 1H) 1.86 (d, J=5.52 Hz, 1H) 1.53 (d, J=6.62 Hz, 3H); MS (ESI) m/z 338 (M+H)⁺.

### EXAMPLE 95E

### 2-{1-Methyl-3-[2-(4-phenoxy-phenoxy)-thiazol-5-yl]-prop-2-ynyl}-isoindole-1,3-dione

To a solution of 4-[2-(4-phenoxy-phenoxy)-thiazol-5-yl]-but-3-yn-2-ol (Example 95D, 3.4 g, 0.01 mol), phthalimide (1.5 g, 0.01 mol) and triphenylphosphine (3.95 g, 0.015 mol) in THF was added DEAD (2.7 g, 0.015 mol) dropwise at RT. The reaction mixture was concentrated and the crude was filtered through a pad of silica gel eluting with methylenechloride to give 2.57 g of the product as a yellow oil (54% yield). 1H NMR (300 MHz, CHLOROFORM-D) δ 1.76 (d, J=6.99 Hz, 3H) 5.41 (q, J=7.35 Hz, 1H) 6.95-7.45 (m, 10H) 7.67-7.79 (m, 2H) 7.80-8.03 (m, 2H). MS (ESI), M/Z:497.2 (M+MeOH—H)⁺.

### EXAMPLE 95F

### 1-Methyl-3-[2-(4-phenoxy-phenoxy)-thiazol-5-yl]-prop-2-ynylamine

To a solution of 2-{1-methyl-3-[2-(4-phenoxy-phenoxy)-thiazol-5-yl]-prop-2-ynyl}-isoindole-1,3-dione (Example 95E, 2.4 g, 0.0053 mol) in a mixture of methylene chloride and methanol was added hydrazine monohydrate (2.6 g, 0.052 mol) and the reaction mixture was reflux under nitrogen for 2 h. The suspension was diluted with methylene chloride and washed with 10% K₂CO₃, brine and dried over magnesium sulfate. The filtrate was concentrated to give 2.02 g of the crude as a yellow oil, which solidified upon standing. And used without further purification. 1H NMR (300 MHz, CHLOROFORM-D) δ ppm 1.41 (d, J=6.99 Hz, 3H) 3.90 (q, J=6.62 Hz, 1H) 6.98-7.44 (m, 10H). MS (ESI), M/Z:320.0 (M—NH₃+H)⁺.

### EXAMPLE 95G

### {1-Methyl-3-[2-(4-phenoxy-phenoxy)-thiazol-5-yl]-prop-2-ynyl}-carbamic acid 4-nitro-phenyl ester

To a solution of 1-methyl-3-[2-(4-phenoxy-phenoxy)-thiazol-5-yl]-prop-2-ynylamine (Example 95F, 0.68 g, 2 mmol) in methylene chloride (20 mL) was added pyridine (0.8 g, 10 mmol), followed by 4-nitrophenylchloroformate (0.45 g, 2.2 mmol) and the reaction mixture was stirred at RT for 2 h. LC-MS indicated the completion. Diluted with methylene chloride and washed with 0.2 N HCl solution, brine and dried over magnesium sulfate. The filtrate was concentrated and purified on silica gel (EA/hex, 5˜35%) to give 0.65 g of product as a pale white solid (65% yield). 1H NMR (300 MHz, CHLOROFORM-D) δ ppm 1.58 (d, J=6.99 Hz, 3H) 4.71-4.96 (m, 1H) 5.38 (d, J=8.46 Hz, 1H) 6.96-7.53 (m, 12H) 8.21-8.34 (m, 2H). MS (ESI), M/Z:380.1 (M-p-nitrophenol+NH₃)⁺.

### EXAMPLE 95

### N-Hydroxy-N′-{1-methyl-3-[2-(4-phenoxyphenoxy)-1,3-thiazol-5-yl]prop-2-ynyl}urea

To a suspension of {1-methyl-3-[2-(4-phenoxy-phenoxy)-thiazol-5-yl]-prop-2-ynyl}-carbamic acid 4-nitro-phenyl ester (Example 95F, 0.15 g, 0.3 mmol) in acetonitrile was added tert-butyldimethylsilyl hydroxyamine (0.07 g, 0.45 mmol) and the reaction mixture was heated reflux for 4 h. Solvent was evaporated and the residue was purified on silica gel (EA/hex, 50-90%) to give 0.15 g ofthe following product as awhite solid (100% yield). 1H NMR (300 MHz, DMSO-D6) δ 1.39 (d, J=6.99 Hz, 3H) 3.17 (d, J=5.15 Hz, 1H) 4.78 (dd, J=8.64, 7.17 Hz, 1H) 6.90-7.66 (m, 10H) 8.51 (s, 1H) 8.61 (s, 1H). MS (ESI), M/Z:396.1 (M+H)⁺.

### EXAMPLE 96

### N-{1-methyl-3-[2-(4-phenoxyphenoxy)-1,3-thiazol-5-yl]prop-2-ynyl}propanamide

The mixture of Example 75C (17 mg, 0.05 mmol), propionic acid (7 μL, 0.1 mmol), TBTU (21 mg, 0.065 mmol), and diisopropylethylamine (26 μL, 0.15 mmol) in DMF was stirred at room temperature for 2 hour and purified by HPLC to get 12 mg titled compound (yield 65%). ¹H NMR (300 MHz, DMSO-D₆) δ 8.32 (d, J=7.8 Hz, 1H), 7.45-7.38 (m, 4H), 7.20-7.03 (m, 6H), 4.87-4.78 (m, 1H), 2.08 (q, J=7.8 Hz, 2H), 1.33 (d, J=7.1 Hz, 3H), 0.99 (t, J=7.5 Hz, 3H). MS (ESI) positive ion 393(M+H)⁺; negative ion 391 (M−H)⁻.

### EXAMPLE 97

### N-methyl-N′-{1-methyl-3-[2-(4-phenoxy)phenoxy)-1,3-thiazol-5-yl]prop-2-ynyl}urea

Methyl isocyanate (4 mg, 0.075 mmol) was added to a solution Example 75C (17 mg, 0.01 mmol) in THF (500 μL). The reaction mixture was stirred at room temperature for 10 min. It was diluted with ethyl acetate and purified by flash chromatography on silica gel column with ethyl acetate to provide 18mg titled compound (yield 92%). ¹H NMR (300 MHz, DMSO-D₆) δ 7.50-7.03 (m. 10H), 6.41 (d, J=8.1 Hz, 1H), 5.73 (q, J=4.7 Hz, 1H), 4.72-4.61 (m, 1H), 2.55 (d, J=4.4 Hz, 1H), 1.32 (d, J=7.1 Hz, 3H). MS (ESI) positive ion 394 (M+H)⁺; negative ion 392 (M−H)⁻.

### EXAMPLE 98

### N-(3-{2-[4-(2-methoxyethoxy)phenoxy]-1,3-thiazol-5-yl}-1-methylprop-2-ynyl)acetamide

The titled compound was prepared according to the procedure described in Example 11C, substituting 2-methoxy-ethanol for cyclopentyl-methanol. ¹H NMR (300 MHz, DMSO-D₆) δ 8.38 (d, J=7.7 Hz, 1H), 7.47 (s, 1H), 7.31 (d, J=9.2 Hz, 2H), 7.03 (d, J=9.2 Hz, 2H), 4.85-4.75 (m, 1H), 4.13-4.10 (m, 2H), 3.68-3.64 (m, 2H), 3.31(s, 3H), 1.81 (s, 3H), 1.32 (d, J=7.1 Hz, 3H). MS (ESI) positive ion 361 (M+H)⁺; negative ion 359 (M−H)⁻.

### EXAMPLE 99

### 3,4-dimethyl-N-{1-methyl-3-[2-(4-phenoxyphenoxy)-1,3-thiazol-5-yl]prop-2-ynyl}isoxazol-5-amine

### EXAMPLE 99A

### N-(3,4-Dimethyl-isoxazol-5-yl)-4-nitro-benzenesulfonamide

The title compound was obtained by using the same procedure as described for Example 89A in 88% yield. 1H NMR (300 MHz, DMSO-D6) δ ppm 1.68 (s, 3H) 2.10 (s, 3H) 7.85-8.18 (m, 2H) 8.35-8.72 (m, 2H). MS (ESI), M/Z:295.8 (M−H)⁺.

### EXAMPLE 99B

### N-(3,4-Dimethyl-isoxazol-5-yl)-N-{1-methyl-3-[2-(4-phenoxy-phenoxy)-thiazol-5-yl]-prop-2-ynyl}-4-nitro-benzenesulfonamide

The title compound was synthesized by using the same procedure as described for Example 89B in 33% yield. 1H NMR (300 MHz, CHLOROFORM-D) δ ppm 1.37 (d, J=6.99 Hz, 3H) 2.00 (s, 3H) 2.29 (s, 3H) 5.25 (d, J=6.99 Hz, 1H) 6.94-7.49 (m, 10H) 7.95-8.11 (m, 2H) 8.24-8.38 (m, 2H). MS (ESI), M/Z:616.8 (M+H)⁺.

### EXAMPLE 99

### 3,4-dimethyl-N-{1-methyl-3-[2-(4-phenoxyphenoxy)-1,3-thiazol-5-yl]prop-2-ynyl}isoxazol-5-amine

The title compound was synthesized by using the same procedure as described for Example 89 in 74% yield. 1H NMR (300 MHz, DMSO-D6) δ ppm 1.47 (d, J=6.99 Hz, 3H) 1.73 (s, 3H) 1.99 (s, 3H) 4.62 (dd, J=8.46, 6.99 Hz, 1H) 6.94-7.55 (m, 10H). MS (ESI), M/Z:431.9 (M+H)⁺.

### EXAMPLE 100

### N-(3-{2-[(7-methoxy-2-naphthyl)oxy]-1,3-thiazol-5-yl}-1-methylprop-2-ynyl)acetamide

### EXAMPLE 100A

### 5-Bromo-2-(7-methoxy-naphthalene-2-yloxy)-thiazole

2,5-Dibromothiazole (0.610 g, 2.51 mmoL) was dissolved in anhydrous DMF (2.0 mL). This was treated with 7-methoxy-2-naphthol (0.536 g, 3.08 mmoL) followed by potassium carbonate (0.265 g, 1.92 mmoL). The solution was then heated in the microwave at 160° C. for 15 min. The contents of the reaction were then filtered and the DMF removed under high vacuum. The residue was triturated with MeOH to afford the title compound (0.560 g, 66%). MS (CI) m/e 337.9 (M+H); ¹H NMR (300 MHz, CDCl₃) δ ppm 3.92 (s, 3H) 7.05-7.31 (m, 5 H) 7.60 (d, J=2.21 Hz, 1H) 7.78 (dd, J=20.59, 8.82 Hz, 1H).

### EXAMPLE 100

### N-(3-{2-[(7-methoxy-2-naphthyl)oxy]-1,3-thiazol-5-yl}-1-methylprop-2-ynyl)acetamide

5-Bromo-2-(7-methoxy-naphthalene-2-yloxy)-thiazole (0.219 g, 0.650 mmoL), N-(1-methyl-prop-2-ynyl)acetamide (0.095 g, 0.860 mmoL), and triethylamine (2.5 mL) were dissolved in anhydrous THF (5 mL). The solution was degassed then it was treated with dichlorobis(triphenylphosphine)palladium (II) (0.22 g, 0.031 mmoL) followed by copper iodide (0.003 g, 0.016 mmoL). It was then heated to 80° C. for 2 hr. More N-(1-methyl-prop-2-ynyl)acetamide (0.030 g, 0.270 mmoL) and dichlorobis(triphenylphosphine)palladium (II) (0.005 g, 0.007 mmoL) were added and heating was continued for an additional 1 hr. The solvent was removed in vacuo. The crude product was purified by flash chromatography on SiO₂ eluting with a solvent gradient from 30% to 80% EtOAc in hexanes to afford the title compound (0.033 g, 14%). MS (CD) m/e 367.1(M+H); ¹H NMR (300 MHz, CDCl₃) δ ppm 7.78 (dd, J=20.22, 8.82 Hz, 1H) 7.62 (d, J=2.21 Hz, 1H) 7.07-7.37 (m, 5H) 5.64 (d, 1H) 4.95-5.13 (m, 1H) 3.92 (s, 3H) 1.55 (s, 3H) 1.46 (d, J=6.99 Hz, 3H).

### EXAMPLE 101

### N-methoxy-N′-{1-methyl-3-[2-(4-phenoxyphenoxy)-1,3-thiazol-5-yl]prop-2-ynyl}urea

To a suspension of {1-methyl-3-[2-(4-phenoxy-phenoxy)-thiazol-5-yl]-prop-2-ynyl}-carbamic acid 4-nitro-phenyl ester (Example 95C, 0.05 g, 0.1 mmol), methoxyamine hydrochloride (0.02 g, 0.2 mmol) in acetonitrile was added triethylamine (500 uL) and the mixture was stirred at room temperature for 4 hour. Solvent was removed and the crude was purified on silica gel (EtOAc/Hex, 30˜75%) to give 0.028 g of product as a white solid (68% yield). 1H NMR (300 MHz, DMSO-D6) δ ppm 1.39 (d, J=6.99 Hz, 3H) 3.51 (s, 3H) 4.38-5.09 (m, 1H) 6.97-7.46 (m, 10H) 7.49 (s, 1H) 9.23 (s, 1H). MS (ESI), M/Z:410.0 (M+H)⁺.

### EXAMPLE 102

### N-[3-(2-{4-[(5-bromo-1,3-thiazol-2-yl)oxy]phenoxy}-1,3-thiazol-5-yl)-1-methylprop-2-ynyl]acetamide

### EXAMPLE 102A

A solution of 2,5-dibromothiazole (3 g, 12.35 mmol), and hydroquinone (2.04 g, 18.53 mmol) in DMF (30mL) was treated with potassium carbonate (1.71 g, 12.35 mmol) and the mixture was heated at 130° C. for 3 hour. The reaction was cooled to 25° C., poured into water (200 mL) and extracted with diethyl ether (3×150 mL). The combined organic layers were washed with water (3×150 mL) and brine (150 mL), dried over sodium sulfate and concentrated on a rotary evaporator. The crude concentrate (3.2 g) was purified by flash chromatography on silica gel eluting with a solvent gradient from 1% to 5% methanol in chloroform to provide 200 mg (5%) of Example 102A. ¹H NMR (300 MHz, DMSO-D6) δ ppm 7.46 (s, 2H) 7.50 (s, 4H)

### EXAMPLE 102B

### N-[3-(2-{4-[(5-bromo-1,3-thiazol-2-yl)oxy]phenoxy}-1,3-thiazol-5-yl)-1-methylprop-2-ynyl]acetamide

A solution of Example 102A (60 mg, 0.139 mmol), Example 5B (15.4 mg, 0.139 mmol), Et₃N (67.5 μL, 0.485 mmol) and THF (2 mL) was degassed and treated with Pd(PPh₃)₂Cl₂ (4.2 mg, 0.006 mmol) and CuI (0.57 mg, 0.003 mmol). The reaction was heated under nitrogen at 80° C. for 2 hour. The solvent was evaporated and the crude concentrate was purified by reverse-phase HPLC on an Atlantis C18 column (1.9×10 cm, 5 μm particle size) using a gradient of 5% to 100% acetonitrile:0.1% aqueous TFA to provide 15 mg (23%) of Example 102B.

1H NMR (300 MHz, DMSO-D6) δ ppm 1.34 (d, J=6.99 Hz, 3H) 1.82 (s, 3H) 4.66-4.96 (m, 1 H) 7.46 (s, 1H) 7.50 (s, 5H) 8.42 (d, J=8.09 Hz, 1H); HRMS (M+H)⁺, m/z, (C₁₈H₁₅BrN₃O₃S₂): calcd:463.9733; found:463.9744.

### EXAMPLE 103

### N-(3-{2-[(4-isopropoxyphenyl)thio]-1,3-thiazol-5-yl}-1-methylprop-2-ynyl)acetamide

### EXAMPLE 103A

### 4-(5-Bromo-thiazol-2-ylsulfanyl)-phenol

A solution of 4-mercaptophenol (300 mg, 2.38 mmol) and 2,5-dibromothiazole (635 mg, 2.61 mmol) in DMF (5 mL) was treated with K₂CO₃ (290 mg, 2.1 mmol) and the resulting mixture was heated at 90° C. for 20 min. The reaction was cooled to 25° C., diluted with water (20 mL) and extracted with 1:1 EtOAc/Et₂O (100 mL). The organic layer was washed with water (3×25 mL), and brine (25 mL), dried (Na₂SO₄), filtered, and evaporated. The crude product was purified by flash chromatography on silica gel eluting with a solvent gradient from 10% to 25% EtOAc in hexanes to provide 586 mg (85%) of Example 103A as a white solid.

1H NMR (300 MHz, DMSO-D6) δ ppm 6.91 (d, J=8.82 Hz, 2H) 7.54 (d, J=8.82 Hz, 2H) 7.75 (s, 1H) 10.23 (br.s, 1H); MS (ESI) m/z 289.8 (M+H)⁺.

### EXAMPLE 103B

### N-{3-[2-(4-Hydroxy-phenylsulfanyl)-thiazol-5-yl]-1-methyl-prop-2-ynyl}-acetamide

Example 103B was prepared according to the procedure described in Example 81B substituting Example 103A for Example 81A. The crude product was purified by flash chromatography on silica gel eluting with a solvent gradient from 1% to 5% MeOH in CHCl₃ to provide Example 103B (63%) as a white solid. ¹H NMR (300 MHz, DMSO-D6) δ ppm 1.30 (d, J=6.99 Hz, 3H) 1.79 (s, 3H) 4.57-4.91 (m, 1H) 6.91 (d, J=8.46 Hz, 2H) 7.54 (d, J=8.46 Hz, 2H) 7.80 (s, 1H) 8.37 (d, J=7.72 Hz, 1H) 10.23 (br.s, 1H); MS (ESI) m/z 319.0 (M+H)⁺.

### EXAMPLE 103C

### N-(3-{2-[(4-isopropoxyphenyl)thio]-1,3-thiazol-5-yl}-1-methylprop-2-ynyl)acetamide

A solution of Example 103b (50 mg, 0.157 mmol), Ph₃P (57.5 mg, 0.22 mmol), and 2-propanol (17.25 μL, 0.252 mmol) in THF (0.5 mL) was treated with diethyl azodicarboxylate (40 μL, 0.22 mmol) dropwise over 2 min. The reaction was stirred at 25° C. for 12 h and concentrated. The crude product was purified by reverse-phase HPLC on an Atlantis C18 column (1.9×10 cm, 5 μm particle size) using a gradient of 5% to 100% acetonitrile:0.1% aqueous TFA to provide 10 mg (18%) of Example 103c as a pale yellow oil. ¹H NMR (300 MHz, DMSO-D6) δ ppm 1.30 (d, J=6.25 Hz, 9H) 1.79 (s, 3H) 4.64-4.76 (m, 1H) 4.74-4.92 (m, 1H) 7.07 (d, J=8.82 Hz, 2H) 7.63 (d, J=8.82 Hz, 2H) 7.82 (s, 1H) 8.37 (d, J=7.72 Hz, 1 H); MS (ESI) m/z 361.0 (M+H)⁺.

### EXAMPLE 104

### 4-({5-[3-(acetylamino)but-1-ynyl]-1,3-thiazol-2-yl}oxy)phenyl pyrrolidine-1-carboxylate

A solution of Example 81B (35 mg, 0.116 mmol) in THF (1 mL) was treated with Et₃N (40.3 μL, 0.29 mmol), and 4-(dimethylamino)-pyridine (1.4 mg, 0.012 mmol). 1-pyrrolidinecarbonyl chloride (25.6 μL, 0.232 mmol) was added and the resulting mixture was heated at 55° C. for 8 h. The reaction was concentrated and the crude product was purified by reverse-phase HPLC on an Atlantis C18 column (1.9×10 cm, 5 μm particle size) using a gradient of 5% to 100% acetonitrile:0.1% aqueous TFA to provide 10 mg (22%) of Example 104 as a yellow oil. ¹H NMR (300 MHz, DMSO-D6) δ ppm 1.33 (d, J=6.99 Hz, 3H) 1.81 (s, 3H) 1.83-1.98 (m, 4H) 3.34 (t, J=6.62 Hz, 2H) 3.50 (t, J=6.62 Hz, 2H) 4.66-4.93 (m, 1H) 7.24 (d, J=9.19 Hz, 2H) 7.39 (d, J=9.19 Hz, 2H) 7.49 (s, 1H) 8.42 (d, J=7.72 Hz, 1H); MS (ESI) m/z 400.1 (M+H)⁺.

### EXAMPLE 105

### 4-[2-(4-phenoxy)phenoxy)-1,3-thiazol-5-yl]but-3-yn-1-ol

The titled compound was prepared according to the procedure described in Example 34, substituting 4-phenoxy-phenol for 3-phenoxy-phenol and but-3-yn-1-ol for but-3-yn-2-ol. ¹H NMR (300 MHz, DMSO-D₆) δ 7.46-7.01 (m, 10H), 4.91 (t, J=5.6 Hz, 1H), 3.55 (q, J=5.6 Hz, 2H), 2.57 (t, J=5.6 Hz, 2H). MS (ESI) positive ion 338 (M+H)⁺; negative ion 336 (M−H)⁻.

### EXAMPLE 106

### methyl 1-methyl-3-[2-(4-phenoxyphenoxy)-1,3-thiazol-5-yl]prop-2-ynylcarbamate

To a solution of Example 75C (7 mg, 0.02 mmol), diisopropylethylamine (10 μL, 0.06 mmol) in dichloromethane (500 μL) was added methyl chloroformate (2.3 uL, 0.03 mmol). The reaction mixture was stirred at room temperature for 10 min. It was diluted with ethyl acetate and purified by flash chromatography on silica gel column to get 8 mg titled compound (yield 100%). ¹H NMR (300 MHz, DMSO-D₆) δ 7.80-7.73 (m, 1H), 7.50 (s, 1H), 7.43-7.03 (m. 9H), 4.64-4.53 (m, 1H), 3.55 (s, 3H), 1.35 (d, J=7.1 Hz, 3H). MS (ESI) 395 (M+H)⁺.

### EXAMPLE 107

### 2-cyano-N-{1-methyl-3-[2-(4-phenoxyphenoxy)-1,3-thiazol-5-yl]prop-2-ynyl}acetamide

The titled compound was prepared according to the procedure described in Example 96, substituting cyano-acetic acid for propionic acid. ¹H NMR (300 MHz, DMSO-D₆) δ 8.85 (d, J=7.8 Hz, 1H), 7.52 (s, 1H), 7.45-7.02 (m, 9H), 4.87-4.78 (m, 1H), 3.66 (d, J=4.1 Hz, 2H), 1.37 (d, J=6.8 Hz, 3H). MS (ESI) positive ion 404 (M+H)⁺; negative ion 402 (M−H)⁻.

### EXAMPLE 108

### N-{1-methyl-3-[2-(4-phenoxy)phenoxy)-1,3-thiazol-5-yl]prop-2-ynyl}-1,3-thiazol-2-amine

### EXAMPLE 108A

### Thiazol-2-yl-carbamic acid tert-butyl ester

A mixture of 2-aminothiazole (5.0 g, 0.05 mol), Boc anhydride (11.35 g, 0.052 mol) and DMAP (10 mg) was stirred at RT overnight. The precipitate was collected by filtration, washed with hexane, air-dried to give 4.8 g of product as a white solid. The filtrate was concentrated and partitioned between ethyl acetate and water. Organic layer was washed with brine, dried over magnesium sulfate and concentrated. The crude was triturated with ethyl acetate/hexane to give 3.8 g of more product (total 8.6 g, 86% yield). 1H NMR (300 MHz, DMSO-D6) δ ppm 1.48 (s, 9H) 3.33 (s, 1H) 7.14 (d, J=3.68 Hz, 1H) 7.36 (d, J=3.68 Hz, 1H) 11.41 (s, 1H). MS (ESI), M/Z 201. (+H)⁺.

### EXAMPLE 108B

### (1-Methyl-prop-2-ynyl)-thiazol-2-yl-carbamic acid tert-butyl ester

A solution of thiazol-2-yl-carbamic acid tert-butyl ester (Example 108A, 0.5 g, 0.0025 mol), 3-butyn-2-ol (0.18 mL, 0.0025 mol) and triphenylphosphine (0.98 g, 0.0038 mol) in dry THF was added a solution of DEAD (0.67 g, 0.0038 mol) was added and the reaction mixture was stirred at RT for 2 h. Solvent was removed and the residue was purified by chromatography on silica gel to give 0.44 g of product as a white crystal (70% yield). 1H NMR (300 MHz, CHLOROFORM-D) δ ppm 1.61 (s, 9H) 1.67 (d, J=6.99 Hz, 3H) 2.30 (d, J=2.57 Hz, 1 H) 6.10 (ddd, J=13.97, 6.99, 2.57 Hz, 1H) 6.95 (d, J=3.31 Hz, 1H) 7.43 (d, J=3.68 Hz, 1H). MS (ESI), M/Z:144.9 (M-^{t}BuOH+H₂O)⁺.

### EXAMPLE 108C

### {1-Methyl-3-[2-(4-phenoxy-phenoxy)-thiazol-5-yl]-prop-2-ynyl}-thiazol-2-yl-carbamic acid tert-butyl ester

A solution of 5-iodo-2-(4-phenoxy-phenoxy)-thiazole (0.36 g, 0.0009 mol), (1-methyl-prop-2-ynyl)-thiazol-2-yl-carbamic acid tert-butyl ester (Example 108B, 0.25 g, 0.001 mol), copper iodide (0.02 g,0.1 mmol) and triethylamine (0.46 g, 0.0045 mol) in dry DMF was degassed for 5 min. After purging with nitrogen, tetrakis(triphenylphosphine)-palladium (58 mg, 0.05 mmol) was added and the reaction mixture was heated at 65° C. over the weekend. Water was added and extracted with ethyl acetate. Organic phase was washed with brine, dried over magnesium sulfate and filtered. The filtrate was concentrated and purified by chromatography on silica gel to give 0.12 g of product as an oil (23% yield). 1H NMR (300 MHz, CHLOROFORM-D) δ ppm 1.61 (s, 9H) 1.72 (d, J=6.99 Hz, 3H) 6.34 (q, J=6.99 Hz, 1H) 6.97 (d, J=3.68 Hz, 1H) 6.99-7.39 (m, 10H) 7.43 (d, J=3.68 Hz, 1H). MS (ESI), M/Z:464.0 (M-^{t}BuOH+H₂O)⁺.

### EXAMPLE 108

### {1-Methyl-3-[2-(4-phenoxy-phenoxy)-thiazol-5-yl]-prop-2-ynyl}-thiazol-2-yl-amine

To a solution of {1-methyl-3-[2-(4-phenoxy-phenoxy)-thiazol-5-yl]-prop-2-ynyl}-thiazol-2-yl-carbamic acid tert-butyl ester (0.1 g, 0.19 mmol) in methylene chloride was added TFA dropwise. The reaction mixture was stirred at room temperature overnight. Solvent was removed and the residue was triturated with ether to give 89 mg of product as a white solid (88% yield). 1H NMR (300 MHz, DMSO-D6) δ ppm 1.51 (d, J=6.99 Hz, 3H) 4.69-5.08 (m, 1H) 6.76 (d, J=3.68 Hz, 1H) 6.90-7.59 (m, 11H) 8.57 (s, 1H). Calcd. Anal. for C₂₄H₁₈F₃N₃O₄S₂: C, 54.03;H, 3.40; N, 7.88; F, 10.68. Found: C, 53.88;H, 3.45; N, 7.75; F, 10.51.

### EXAMPLE 109

### N-{3-[2-(4-isopropoxyphenoxy)-1,3-thiazol-5-yl]-1-methylprop-2-ynyl}acetamide

The titled compound was prepared according to the procedure described in Example 11C, substituting propan-2-ol for cyclopentyl-methanol. ¹H NMR (300 MHz, DMSO-D₆) δ 8.40 (d, J=7.8 Hz, 1H), 7.47 (s, 1H), 7.29 (d, J=9.2 Hz, 2H), 7.01 (d, J=9.2 Hz, 2H), 4.85-4.75 (m, 1H), 4.66-4.55 (m, 1H), 1.81 (s, 3H), 1.32 (d, J=7.1 Hz, 3H), 1.27 (d, J=6.1 Hz, 6H). MS (ESI) positive ion 345 (M+H)⁺; negative ion 343 (M−H)⁻.

### EXAMPLE 110

### 5-methyl-N-{1-methyl-3-[2-(4-phenoxyphenoxy)-1,3-thiazol-5-yl]prop-2-ynyl}isoxazol-3-amine

### EXAMPLE 110A

### N-(5-Methyl-isoxazol-3-yl)-4-nitro-benzenesulfonamide

To a solution of 3-amino-5-methylisoxazole (2.0 g, 0.02 mol) in pyridine (10 mL) at 0° C. was added p-nitrobenzenesulfonyl chloride (4.9 g, 0.022 mol) in portions. The reaction mixture was stirred overnight while warming up to room temperature. Water was added and stirring was continued for 20 min. Yellow solid precipitated out and was filtered, ari-dired to give 6.7 g crude product, which was recrystallized from ethyl acetate and hexane to 4.22 g of product as a brown crystal (74% yield). 1H NMR (300 MHz, DMSO-D6) δ ppm 2.30 (s, 3H) 6.16 (s, 1H) 7.97-8.21 (m, 2H) 8.28-8.60 (m, 2H) 11.85 (s, 1H). MS (ESI), M/Z:281.8 (M−H)⁺.

### EXAMPLE 110B

### N-(5-Methyl-isoxazol-3-yl)-N-{1-methyl-3-[2-(4-phenoxy-phenoxy)-thiazol-5-yl]-prop-2-ynyl}-4-nitro-benzenesulfonamide

To a suspension of N-(5-methyl-isoxazol-3-yl)-4-nitro-benzenesulfonamide (Example 110A, 0.57 g, 2 mmol), polymer-bound TPP (0.79 g, 3 mmol) and 4-[2-(4-phenoxy-phenoxy)-thiazol-5-yl]-but-3-yn-2-ol (0.67 g, 2 mmol) in dry THF was added a solution of DEAD (0.53 g, 3 mmol) dropwise at room temperature. The mixture was stirred for 4 hours. Then additional DEAD (0.1 mL) was added and the color changed to very dark brown. After 16 hours, the mixture was filtered through Celite and the filtrate was concentrated and purified on silica gel (ethyl acetate/hexane, 5˜35%) to give 0.76 g of the desired as a semisolid (63% yield). 1H NMR (300 MHz, CHLOROFORM-D) δ ppm 1.53 (d, J=6.99 Hz, 3H) 2.46 (s, 3H) 5.40 (t, J=6.99 Hz, 1H) 6.22 (s, 1H) 6.91-7.50 (m, 10H) 7.97-8.14 (m, 2H) 8.20-8.42 (m, 2H). MS (ESI), M/Z:602.8 (M+H)⁺.

### EXAMPLE 110

### 5-methyl-N-{1-methyl-3-[2-(4-phenoxyphenoxy)-1,3-thiazol-5-yl]prop-2-ynyl}isoxazol-3-amine

The title compound was synthesized by using the same procedure as described for Example 2. 1H NMR (300 MHz, DMSO-D6) δ ppm 1.44 (d, J=6.99 Hz, 3H) 2.23 (s, 3H) 4.33-4.49 (m, 1H) 5.67 (s, 1H) 6.52 (d, J=8.46 Hz, 1H) 6.89-7.62 (m, 10H) 6.94-7.56 (m, 6H). MS (ESI), M/Z:417.8 (M+H)⁺.

### EXAMPLE 111

### 2-methyl-N-{1-methyl-3-[2-(4-phenoxyphenoxy)-1,3-thiazol-5-yl]prop-2-ynyl}propanamide

The titled compound was prepared according to the procedure described in Example 96, substituting isobutyric acid for propionic acid. ¹H NMR (300 MHz, DMSO-D₆) δ 8.28 (d, J=7.8 Hz, 1H), 7.49(s, 1H), 7.45-7.38 (m, 4H), 7.20-7.03 (m, 5H), 4.87-4.78 (m, 1H), 2.40-2.30 (m, 1H), 1.33 (d, J=6.8 Hz, 3H), 0.99 (d, J=6.8 Hz, 6H). MS (ESI) positive ion 407 (M+H)⁺; negative ion 405 (M−H)⁻.

### EXAMPLE 112

### N-{3-[2-(4-isopropoxyphenoxy)-1,3-thiazol-5-yl]-1-methylprop-2-ynyl}urea

To a solution of 3-[2-(4-Isopropoxy-phenoxy)-thiazol-5-yl]-1-methyl-prop-2-ynylamine (Example 92C, 82 mg, 0.27 mmole) in methylene chloride (4 mL) at 0° C., was added trichloromethyl isocyanate (40 uL, 0.4 mmole). After 15 minutes, the reaction mixture was diluted with methylene chloride (35 mL) and washed with 10% aqueous potassium carbonate followed by brine. The reaction mixture was dried over magnesium sulfate filtered and evaporated. The resulting mixture was added methanol and heated at reflux for 4.5 hours. Solvent was evaporated and upon addition of ether solid precipitated. The solid was filtered and washed in turn, with ether and hexane to yield 38.0 mg of the product as off-white solid. 1H NMR (300 MHz, CHLOROFORM-D) δ ppm 7.29 (s, 1H) 7.10-7.21 (m, 2H) 6.83-6.96 (m, 2H) 4.80 (q, J=6.99 Hz, 1H) 4.42-4.61 (m, 1H) 1.47 (d, J=6.62 Hz, 3H) 1.34 (d, J=6.25 Hz, 6H), MS (ESI) M/Z 346 (M+1)⁺.

### EXAMPLE 113

### N-{3-[2-(3,4-dimethylphenoxy)-1,3-thiazol-5-yl]-1-methylprop-2-ynyl}acetamide

A Smith Process vial (0.5-2 ml) was charged with a stir bar. To the vessel were added dibromothiazole(36 mg, 0.15 mmol) and 3,4-dimethylphenol (0.16 mmol). MP-Carbonate (0.45 mmol, 3 mmol/g loading) was added to above solution. The reaction vessel was sealed and heated in microwave to 150° C. for 1200 s. After cooling, the reaction vessel was uncapped. Without further work-up, triethylamine (63 μl, 0.45 mmol) and alkyne (18 mg, 0.16 mmol) were added to the resulting mixture followed by PdCl₂(PPh₃)₂ (5.3 mg, 0.0075 mmol) and CuI (1 mg, 0.0045 mmol). The resulting reaction mixture was capped and heated in microwave to 150° C. for another 1200 s. After cooling, the vessel was again uncapped and filtered through a small plug of silica gel. The filtrate was collected and dried. HPLC purification gave the desired products. ¹H NMR δ; 1.32 (d, 3H, J=7.1 Hz), 1.81 (s, 3H), 2.23 (s, 3H), 2.24 (s, 3H), 4.81 (m, 1H),7.08 (dd, 1H, J=8.2, 2.5 Hz) 7.16 (d, 1H, J=2.5 Hz), 7.32 (d, 1H, J=8.2 Hz), 7.47 (s, 1H), 8.37 (d, 1H, 7.8 Hz); MS (ESI) m/z 314.9 (M+H)

### EXAMPLE 114

### N-(3-{2-[4-(isopropylamino)phenoxy]-1,3-thiazol-5-yl}-1-methylprop-2-ynyl)acetamide

Example 54 (2.85 g, 7.0 mmol) was dissolved in CH₂Cl₂ (35 ml) and TFA (8.75 ml, 25% v/v) was added via syringe. After 4 h of stirring at rt, the reaction solution was concentrated by rotary evaporation and then placed on high vacuum for 2 h to give a light yellow solid which was used in the next step without further purification. The crude product from above (500 mg, 1.7 mmol) was dissolved in a buffer solution prepared from NaOAc/HOAc in MeOH (6 ml, see Example 47). To this solution was added acetone (0.374 ml, 5.1 mmol) followed by NaCNBH₃ (320 mg, 5.1 mmol). The reaction was heated to 70° C. in a water bath for 1 h. The reaction solution was cooled and the MeOH was evaporated. The residue was taken up in EtOAc and quenched with water. The layers were separated and the aqueous layer was extracted once with EtOAc. The combined organics were dried (Na₂SO₄), filtered and concentrated by rotary evaporation. The residue was purified by flash column chromatography to give Example 114 as a hydroscopic white solid. MS (ESI) m/z 344.2 (M+H); ¹H NMR (300 MHz, DMSO-d₆) δ ppm 1.06-1.20 (d, J=6.0 Hz, 6H) 1.31 (d, J=6.99 Hz, 3H) 1.80 (s, 3H) 3.52 (m, 1H) 4.70-4.88 (m, 1H) 5.64 (d, J=7.72 Hz, 1H) 6.52-6.65 (d, J=9.0 Hz, 2H) 7.00-7.13 (d, J=9.0 Hz, 2H) 7.46 (s, 1H) 8.39 (d, J=7.72 Hz, 1H).

### EXAMPLE 115

### N-hydroxy-N′-{3-[2-(4-isopropoxyphenoxy)-1,3-thiazol-5-yl]-1-methylprop-2-ynyl}urea

The tilted compound was obtained from the reaction mixture of Example 125 by reverse phase HPLC as a white solid. 1H NMR (300 MHz, CHLOROFORM-D) δ 1.34 (d, J=5.88 Hz, 6H) 1.52 (d, J=6.99 Hz, 3H) 4.45-4.59 (m, 1H) 4.78-4.96 (m, 1H) 6.71 (d, J=8.09 Hz, 1H) 6.85-6.95 (m, 2H) 7.11-7.21 (m, 2H) 7.31 (s, 1H). MS (ESI), M/Z:362.0 (M+H)⁺.

### EXAMPLE 116

### N-{3-[2-(4-ethoxyphenoxy)-1,3-thiazol-5-yl]-1-methylprop-2-ynyl}acetamide

A solution of Example 81B (40 mg, 0.132 mmol), Ph₃P (45 mg, 0.172 mmol), and EtOH (13.4 μL, 0.238 mmol) in THF (0.3 mL) was treated with diethyl azodicarboxylate (36 μL, 0.197 mmol) dropwise over 2 min. The reaction was stirred at 25° C. for 1 h and was concentrated. The crude product was purified by reverse-phase HPLC on an Atlantis C18 column (1.9×10 cm, 5 μm particle size) using a gradient of 5% to 100% acetonitrile:0.1% aqueous TFA to provide 11 mg (25%) of Example 116 as a yellow solid. ¹H NMR (300 MHz, DMSO-D6) δ ppm 8.40 (d, J=7.72 Hz, 1H) 7.48 (s, 1H) 7.31 (d, J=9.19 Hz, 2H) 7.01 (d, J=9.19 Hz, 2H) 4.62-4.93 (m, 1H) 4.04 (q, J=6.99 Hz, 2H) 1.81 (s, 3H) 1.32 (d, J=6.99 Hz, 3H) 1.33 (t, J=6.99 Hz, 3H); HRMS (M+H)⁺, m/z, (C₁₇H₁₉N₂O₃S): calcd:331.1111; found:331.1116.

### EXAMPLE 117

### 2-methoxy-N-{1-methyl-3-[2-(4-phenoxyphenoxy)-1,3-thiazol-5-yl]prop-2-ynyl}acetamide

The titled compound was prepared according to the procedure described in Example 96, substituting methoxyacetic acid for propionic acid. ¹H NMR (300 MHz, DMSO-D₆) δ 8.34 (d, J=7.8 Hz, 1H), 7.50(s, 1H), 7.45-7.38 (m, 4H), 7.20-7.03 (m, 5H), 4.97-4.87 (m, 1H), 3.82(s, 2H), 3.30(s, 3H), 1.38 (d, J=6.8 Hz, 3H). MS (ESI) positive ion 409 (M+H)⁺; negative ion 407 (M−H)⁻.

### EXAMPLE 118

### N-(3-{2-[(7-methoxy-2-naphthyl)oxy]-1,3-thiazol-5-yl}-1-methylprop-2-ynyl)acetamide

### EXAMPLE 118A

### 5 -Bromo-2-(6-methoxy-naphthalene-2-yloxy)-thiazole

Example 118A was prepared according to the procedure described in Example 100A substituting 6-methoxy-2-naphthol for 7-methoxy-2-naphthol. The crude product was purified by trituration in MeOH to provide the title compound (0.292 g, 45%). MS (CI) m/e 337.9 (M+H); ¹H NMR (300 MHz, CDCl₃) δ ppm 7.75 (dd, J=23.16, 8.82 Hz, 1H) 7.65 (d, J=2.57 Hz, 1H) 7.11-7.41 (m, 5H) 3.93 (s, 3H).

### EXAMPLE 118

### N-(3-{2-[(7-methoxy-2-naphthyl)oxy]-1,3-thiazol-5-yl}-1-methylprop-2-ynyl) acetamide

Example 118 was prepared according to the procedure described in Example 100 substituting 5-Bromo-2-(6-methoxy-naphthalene-2-yloxy)-thiazole for 5-Bromo-2-(7-methoxy-naphthalene-2-yloxy)-thiazole. The crude product was purified by flash chromatography on SiO₂ eluting with a solvent gradient from 30% to 80% EtOAc in hexanes to afford the title compound (0.042 g, 28%). MS (CI) m/e 367.0 (M+H); ¹H NMR (300 MHz, DMSO-d₆) δ ppm 8.42 (d, J=7.72 Hz, 1H) 7.81-8.01 (m, 3H) 7.38-7.55 (m, 3H) 7.24 (dd, J=9.19, 2.57 Hz, 1H) 4.73-4.91 (m, 1H) 3.89 (s, 3H) 1.81 (s, 3H) 1.32 (d, J=6.99 Hz, 3H).

### EXAMPLE 119

### N-{3-[2-(2,5-dimethylphenoxy)-1,3-thiazol-5-yl]-1-methylprop-2-ynyl}acetamide

A Smith Process vial (0.5-2 ml) was charged with a stir bar. To the vessel were added dibromothiazole(36 mg, 0.15 mmol) and 2,5-dimethylphenol (0.16 mmol). MP-Carbonate (0.45 mmol, 3 mmol/g loading) was added to above solution. The reaction vessel was sealed and heated in microwave to 150° C. for 1200 s. After cooling, the reaction vessel was uncapped. Without further work-up, triethylamine (63 μl, 0.45 mmol ) and Example 5B (18 mg, 0.16 mmol) were added to the resulting mixture followed by PdCl₂(PPh₃)₂ (5.3 mg, 0.0075 mmol) and CuI (1 mg, 0.0045 mmol). The resulting reaction mixture was capped and heated in microwave to 150° C. for another 1200 s. After cooling, the vessel was again uncapped and filtered through a small plug of silica gel. The filtrate was collected and dried. HPLC purification gave the desired products. ¹H NMR δ; 1.32 (d, 3H, J=7.2 Hz), 1.81 (s, 3H), 2.13 (s, 3H), 2.29 (s, 3H), 4.81 (m, 1H),7.08 (d, 1H, J=7.5 Hz) 7.12 (s, 1H), 7.24 (d, 1H, J=7.5 Hz), 7.45 (s, 1H), 8.37 (d, 1H, 7.8 Hz); MS (ESI) m/z 314.9 (M+H).

### EXAMPLE 120

### N-{3-[2-(5-isopropyl-2-methylphenoxy)-1,3-thiazol-5-yl]-1-methylprop-2-ynyl}acetamide

A Smith Process vial (0.5-2 ml) was charged with a stir bar. To the vessel were added dibromothiazole(36 mg, 0.15 mmol) and 3-(2-methyl-ethyl)-6-methylphenol (0.16 mmol). MP-Carbonate (0.45 mmol, 3 mmol/g loading) was added to above solution. The reaction vessel was sealed and heated in microwave to 150° C. for 1200 s. After cooling, the reaction vessel was uncapped. Without further work-up, triethylamine (63 μl, 0.45 mmol) and Example 5B (18 mg, 0.16 mmol) were added to the resulting mixture followed by PdCl₂(PPh₃)₂ (5.3 mg, 0.0075 mmol) and CuI (1 mg, 0.0045 mmol). The resulting reaction mixture was capped and heated in microwave to 150° C. for another 1200 s. After cooling, the vessel was again uncapped and filtered through a small plug of silica gel. The filtrate was collected and dried. HPLC purification gave the desired products. ¹H NMR δ; 1.18 (d, 6H, J=6.0 Hz), 1.32 (d, 3H, J=7.2 Hz), 1.81 (s, 3H), 2.13 (s, 3H), 4.81 (m, 1H), 7.16 (d, 1H, J=7.5 Hz) 7.16 (s, 1H), 7.28 (d, 1H, J=7.5 Hz), 7.45 (s, 1H), 8.37 (d, 1H, 7.8 Hz); MS (ESI) m/z 343.0 (M+H)

### EXAMPLE 121

### N-{1-methyl-3-[2-(2-naphthyloxy)-1,3-thiazol-5-yl]prop-2-ynyl}acetamide

### EXAMPLE 121A

### 5-Bromo-2-(naphthalene-2-yloxy)-thiazole

2,5-Dibromothiazole (0.250 g, 1.03 mmoL) was dissolved in anhydrous DMSO (4.0 mL). This was treated with 2-naphthol (0.156 g, 1.08 mmoL) followed by potassium carbonate (0.144 g, 1.03 mmoL). The solution was then heated in the microwave at 130° C. for 30 minutes. The contents of the reaction were then poured into brine (25 mL) and extracted with CH₂Cl₂ (3×25 mL). The combined extracts were washed with brine (5×25 mL) then dried (Na₂SO₄), filtered, and concentrated in vacuo. The crude product was purified by flash chromatography (SiO₂, 1 EtOAc/19 hexanes) to afford the title compound (0.246 g, 79%). MS (CI) m/e 305.9(M+H); ¹H NMR (300 MHz, CDCl₃) δ ppm 7.19 (s, 1H) 7.37 (dd, J=8.82, 2.21 Hz, 1H) 7.42-7.54 (m, 2H) 7.71 (d, J=2.21 Hz, 1H) 7.75-8.02 (m, 3H)

### EXAMPLE 121

### N-{1-methyl-3-[2-(2-naphthyloxy)-1,3-thiazol-5-yl]prop-2-ynyl}acetamide

5-Bromo-2-(naphthalene-2-yloxy)-thiazole (0.225 g, 0.734 mmoL), N-(1-methyl-prop-2-ynyl)acetamide (0.098 g, 0.880 mmoL), and triethylamine (0.511 mL, 3.67 mmoL) were dissolved in anhydrous CH₃CN (3.8 mL). The solution was degassed then it was treated with dichlorobis(triphenylphosphine)palladium (II) (0.26 g, 0.37 mmoL) followed by copper iodide (0.0035 g, 0.018 mmoL). It was then heated in the microwave at 100° C. for 25 min. and then concentrated in vacuo. The crude product was purified by flash chromatography (SiO₂, 100 EtOAc) to afford the title compound (0.035 g, 14%). MS (CI) m/e 337.1(M+H); ¹H NMR (300 MHz, CDCl₃) δ ppm 1.46 (d, J=6.99 Hz, 3H) 2.00 (s, 3H) 4.97-5.09 (m, 1H) 5.66 (d, J=7.35 Hz, 1H) 7.33 (s, 1H) 7.39 (dd, J=9.01, 2.39 Hz, 1H) 7.46-7.55 (m, 2H) 7.73 (d, J=2.21 Hz, 1H) 7.79-7.93 (m, 3H).

### EXAMPLE 122

### 2-{[({1-methyl-3-[2-(4-phenoxyphenoxy)-1,3-thiazol-5-yl]prop-2-ynyl}amino)carbonyl]amino}acetamide

To a mixture of {1-methyl-3-[2-(4-phenoxy-phenoxy)-thiazol-5-yl]-prop-2-ynyl}-carbamic acid 4-nitro-phenyl ester (Example 95C, 0.05 g, 0.1 mmol) and glycinamide hydrochloride (0.015 g, 0.2 mmol) was added triethylamine (500 uL) and stirred at RT overnight. Solvent was removed and the crude was purified by HPLC to give 0.027 g of product as a white solid (64% yield). 1H NMR (300 MHz, DMSO-D6) δ ppm 1.33 (d, J=6.99 Hz, 3H) 3.60 (d, J=5.15 Hz, 2H) 4.44-4.95 (m, 1H) 6.02 (t, J=5.33 Hz, 1H) 6.69 (d, J=8.09 Hz, 1H) 6.09 (s, 1H) 7.01-7.24 (m, 5H) 7.30 (s, 1H) 7.35-7.47 (m, 4H) 7.49 (s, 1H). MS (ESI), M/Z: 437.0 (M+H)⁺.

### EXAMPLE 123

### tert-butyl 2-{[({1-methyl-3-[2-(4-phenoxyphenoxy)-1,3-thiazol-5-yl]prop-2-ynyl}amino)carbonyl]amino}ethylcarbamate

To a mixture of {1-methyl-3-[2-(4-phenoxy-phenoxy)-thiazol-5-yl]-prop-2-ynyl}-carbamic acid 4-nitro-phenyl ester (Example 95C, 0.1 g, 0.2 mmol) and N-BOC-ethylenediamine (0.07 g, 0.4 mmol) was added triethylamine (500 uL) and stirred at RT overnight. The resulting suspension was filtered and washed with ethyl acetate to give 0.1 g of product as a white solid (95% yield). 1H NMR (300 MHz, CHLOROFORM-D) δ ppm 1.42-1.52 (m, 12H) 3.81-4.00 (m, 2H) 4.67 (d, J=7.72 Hz, 1H) 4.77-4.94 (m, 2H) 6.96-7.49 (m, 10H). MS (ESI), M/Z: 494.0 (M+H)⁺.

### EXAMPLE 124

### 1-methyl-3-[2-(4-phenoxyphenoxy)-1,3-thiazol-5-yl]prop-2-ynyl carbamate

The solution of Example 75C (28 mg, 0.083 mmol) in dicholormethane (500 μL) under nitrogen atmosphere was cooled down to −78° C. Chlorosulfonylisocyanate (22 μL) was added dropwise. The reaction mixture was stirred at −78° C. for 30 min. It was concentrated, dissolved in water (3 mL), and heated to 70° C. with stirring for 45 min. The white solid was filtered and washed with water, dried to get 22 mg titled compound (yield 71%). ¹H NMR (300 MHz, DMSO-D₆) δ 7.57 (s, 1H), 7.47-7.38 (m, 4H), 7.21-7.03 (m, 5H), 6.68 (bs, 2H), 5.49-5.41 (m, 1H), 1.45 (d, J=6.8 Hz, 3H). MS (ESI) positive ion 381 (M+H)⁺; negative ion 379 (M−H)⁻.

### EXAMPLE 125

### N-[(aminooxy)carbonyl]-N-{3-[2-(4-isopropoxyphenoxy)-1,3-thiazol-5-yl]-1-methylprop-2-ynyl}amine

### EXAMPLE 125A

### {3-[2-(4-Isopropoxy-phenoxy-thiazol-5-yl]-1-methyl-prop-2-ynyl}-carbamic acid 4-nitro-phenyl ester

To a suspension of 3-[2-(4-isopropoxy-phenoxy)-thiazol-5-yl]-1-methyl-prop-2-ynylamine (Example 92C, 0.4 g, 0.0013 mol) and pyridine (1.0 g, 0.013 mol) in methylene chloride was added p-nitrophenyl chloroformate (0.28 g, 0.0014 mol) and the mixture was stirred at room temperature for 2 h. Diluted with more CH₂Cl₂ and washed with 0.5 N HCl, brine and dried over magnesium sulfate. After filtration, the filtrate was concentrated to give 0.55 g of product as a light yellow solid (90% yield). 1H NMR (300 MHz, DMSO-D6) δ 6 ppm 1.27 (d, J=5.88 Hz, 6H) 1.45 (d, J=6.99 Hz, 3H) 4.56-4.74 (m, 2H) 6.96-7.03 (m, 2H) 7.26-7.33 (m, 2H) 7.40-7.47 (m, 2H) 7.52 (s, 1H) 8.27 (d, J=9.19 Hz, 2H) 8.70 (d, J=7.72 Hz, 1H). MS (ESI), M/Z:346.1 (M-p-nitropheno+NH₃)⁺.

### EXAMPLE 125

### N-[(Aminooxy)carbonyl]-N-{3-[2-(4-isopropoxyphenoxy)-1,3-thiazol-5-yl]-1-methylprop-2-ynyl}amine

To a mixture of {3-[2-(4-Isopropoxy-phenoxy)-thiazol-5-yl]-1-methyl-prop-2-ynyl}-carbamic acid 4-nitro-phenyl ester (Example 125C, 0.05 g, 0.1 mmol) and hydroxylamine hydrochloride (0.04 g, 0.5 mmol) in acetonitrile was added triethylamine (500 uL) and stirred at RT overnight. Aqueous work-up and reverse phase HPLC purification afforded 0.017 g of product as a white solid. 1H NMR (500 MHz, DMSO-D) δ ppm 1.25 (d, J=5.88 Hz, 6H) 1.38 (d, J=6.99 Hz, 3H) 4.55-4.57 (m, 2H) 6.38 (s, 2H) 6.93-6.97 (m, 2H) 7.23-7.27 (m, 2H) 7.38 (d, J=8.09 Hz, 1H). 7.45 (s, 1H). MS (ESI), M/Z:362.1 (M+H)⁺.

### EXAMPLE 126

### N-{1-methyl-3-[2-(4-phenoxyphenoxy)-1,3-thiazol-5-yl]prop-2-ynyl}-2-furamide

The titled compound was prepared according to the procedure described in Example 96, substituting furan-2-carboxylic acid for propionic acid. ¹H NMR (300 MHz, DMSO-D₆) δ 8.87 (d, J=8.1 Hz, 1H), 7.84-7.82 (m, 1H), 7.51 (s, 1H), 7.45-7.38 (m, 4H), 7.20-7.03 (m, 6H), 6.64-6.62 (m, 1H), 5.12-4.99 (m, 1H), 1.45 (d, J=7.1 Hz, 3H). MS (ESI) 431 (M+H)⁺.

What is claimed is:

1. A compounds of formula (I); 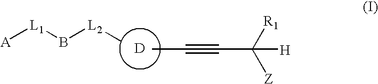
wherein
A is selected from the group consisting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl;
B is selected from the group consisting of an aryl ring and a heteroaryl ring;
D is selected from the group consisting of an aryl ring and a heteroaryl ring;
L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₆ haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}—N═;
R_{c} is selected from the group consisting of hydrogen, alkyl, aryl, haloalkyl, and heteroaryl;
R_{d} and Rₑ are each independently selected from the group consisting of hydrogen, alkyl, haloalkyl, hydroxy and halo, or R_{d} and Rₐ taken together with the atom to which they are attached form oxo;
R_{f} is selected from the group consisting of alkoxy, aryloxy, heteroaryloxy and hydroxy;
R_{g} is H₂N—C(O)— or C₁₋₆ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

2. The compound according to claim 1 wherein
A is selected from the group consisting of alkenyl, alkoxyalkyl and alkyl;
B is selected from the group consisting of an aryl ring and a heteroaryl ring;
D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)2—, —S(O)₂NH—;
L₂ is selected from the group consisting of —C(R_{d}R_{b})—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₆ haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
R_{b} is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₆ alkylHN-C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

3. The compound according to claim 1 wherein
A is selected from the group consisting of alkenyl, alkoxyalkyl and alkyl;
B is selected from the group consisting of an aryl ring and a heteroaryl ring;
D is selected from the group consisting of an aryl ring and a heteroaryl ring; L₁ is absent;
L₂ is selected from the group consisting of —C(R_{d}Rₑ)—, —(CH₂)ₙ—, —NH—, —O—, and —S—;
n is 1, 2 or 3;
Z is a member selected from the group consisting of hydroxy, hydroxyalkyl, R_{g}—O— and Rⱼ—NH—;
R₁ is hydrogen, C₁₋₃ haloalkyl or C₁₋₃ alkyl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

4. The compound according to claim 1 wherein
A is selected from the group consisting of alkenyl, alkoxyalkyl and alkyl;
B is selected from the group consisting of an aryl ring and a heteroaryl ring;
D is selected from the group consisting of an aryl ring and a heteroaryl ring; L₁ is selected from the group consisting of —NR_{c}—, —NR_{c}CH₂—, —O—, and —S—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
R_{c} is selected from the group consisting of hydrogen, alkyl, haloalkyl, hydroxy and halo, or R_{d} and R_{c} taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

5. The compound according to claim 1 wherein
A is selected from the group consisting of alkenyl, alkoxyalkyl and alkyl;
B is selected from the group consisting of an aryl ring and a heteroaryl ring;
D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

6. The compound according to claim 1 wherein
A is selected from the group consisting of alkenyl, alkoxyalkyl and alkyl;
B is selected from the group consisting of an aryl ring and a heteroaryl ring;
D is selected from the group consisting of an aryl ring and a heteroaryl ring; L₁ is —C(RₐR_{b})—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
Rₐ and R_{b} are each individually selected from the group consisting of hydrogen, alkyl, haloalkyl and hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

7. A compound according to claim 1 wherein
A is selected from the group consisting of aryl and arylalkyl,
B is selected from the group consisting of an aryl ring and a heteroaryl ring;
D is selected from the group consisting of an aryl ring and a heteroaryl ring; L₁ is absent;
L₂ is selected from the group consisting of —C(R_{d}Rₑ)—, —(CH₂)ₙ—, —NH—, —O—, and —S—;
n is 1, 2 or 3;
Z is a member selected from the group consisting of hydroxy, hydroxyalkyl, R_{g}—O— and Rⱼ—NH—;
R₁ is hydrogen, C₁₋₃ haloalkyl or C₁₋₃ alkyl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{g} is H₂N—C(O)—or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

8. A compound according to claim 1 wherein
A is selected from the group consisting of aryl, arylalkyl, heteroaryl and heteroarylalkyl;
B is selected from the group consisting of an aryl ring and a heteroaryl ring;
D is selected from the group consisting of an aryl ring and a heteroaryl ring; L₁ is selected from the group consisting of —NR_{c}—, —NR_{c}CH₂—, —O—, and —S—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl—NH—C(O)—, alkoxyalkyl, alkoxyalkylcarbonyl, alkoxycarbonyl, alkoxycarbonyl—NH—alkyl—NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

9. A compound according to claim 1 wherein
A is selected from the group consisting of aryl, arylalkyl, heteroaryl and heteroarylalkyl;
B is selected from the group consisting of an aryl ring and a heteroaryl ring;
D is selected from the group consisting of an aryl ring and a heteroaryl ring;
L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)—or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

10. A compound according to claim 1 wherein
A is selected from the group consisting of aryl, arylalkyl, heteroaryl and heteroarylalkyl;
B is selected from the group consisting of an aryl ring and a heteroaryl ring;
D is selected from the group consisting of an aryl ring and a heteroaryl ring; L₁ is —C(RₐR_{b})—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
Rₐ and R_{b} are each individually selected from the group consisting of hydrogen, alkyl, haloalkyl and hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)—or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH—alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

11. A compound according to claim 1 wherein
A is selected from the group consisting of cycloalkyl, cycloalkylalkyl, heterocycle and heterocyclealkyl;
B is selected from the group consisting of an aryl ring and a heteroaryl ring;
D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl; Rₐ and R_{b} are each individually selected from the group consisting of hydrogen, alkyl, haloalkyl, hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

12. A compound according to claim 1 wherein
A is selected from the group consisting of cycloalkyl, cycloalkylalkyl, heterocycle and heterocyclealkyl;
B is selected from the group consisting of an aryl ring and a heteroaryl ring;
D is selected from the group consisting of an aryl ring and a heteroaryl ring; L₁ is absent;
L₂ is selected from the group consisting of —C(R_{d}Rₑ)—, —(CH₂)ₙ—, —NH—, —O—, and —S—;
n is 1, 2 or 3;
Z is a member selected from the group consisting of hydroxy, hydroxyalkyl, R_{g}—O— and Rⱼ—NH—;
R₁ is hydrogen, C₁₋₃ haloalkyl or C₁₋₃ alkyl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

13. A compound according to claim 1 wherein
A is selected from the group consisting of cycloalkyl, cycloalkylalkyl, heterocycle and heterocyclealkyl;
B is selected from the group consisting of an aryl ring and a heteroaryl ring;
D is selected from the group consisting of an aryl ring and a heteroaryl ring; L₁ is selected from the group consisting of —NR_{c}—, —NR_{c}CH₂—, —O—, and —S—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

14. A compound according to claim 1 wherein
A is selected from the group consisting of cycloalkyl, cycloalkylalkyl, heterocycle and heterocyclealkyl;
B is selected from the group consisting of an aryl ring and a heteroaryl ring;
D is selected from the group consisting of an aryl ring and a heteroaryl ring; L₁ is selected from the group consisting of —C(O)—, —C(O)O—, —C(O)NH—, —NR_{c}(O)—, —NR_{c}C(O)—O—, —NR_{c}S(O)₂—,—OC(O)NH—, —OC(O)—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
Rₑ is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH—alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

15. A compound according to claim 1 wherein
A is selected from the group consisting of cycloalkyl, cycloalkylalkyl, heterocycle and heterocyclealkyl;
B is selected from the group consisting of an aryl ring and a heteroaryl ring;
D is selected from the group consisting of an aryl ring and a heteroaryl ring; L₁ is —C(RₐR_{b})—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
Rₐ and R_{b} are each individually selected from the group consisting of hydrogen, alkyl, haloalkyl, hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N-O—C(O)—, heteroaryl, heteroarylcarbonyl, heterocycle, and heterocyclecarbonyl.

16. A compound according to claim 1 wherein
A is selected from the group consisting of heteroaryl and heteroarylalkyl,
B is selected from the group consisting of an aryl ring and a heteroaryl ring;
D is selected from the group consisting of an aryl ring and a heteroaryl ring; L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—, —NR_{c}C(O)—O—, —NH—N═CH—, —NR_{c}S(O)₂—, —O—, —OC(O)NH—, —OC(O)—, —O—N═CH—, —S—, —S(O)₂—, —S(O)₂NH—;
L₂ is selected from the group consisting of —C(R_{d}Rₑ)—, —(CH₂)ₙ', —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl; Rₐ and R_{b} are each individually selected from the group consisting of hydrogen, alkyl, haloalkyl and hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

17. A compound according to claim 1 wherein
A is selected from the group consisting of heteroaryl and heteroarylalkyl,
B is selected from the group consisting of an aryl ring and a heteroaryl ring;
D is selected from the group consisting of an aryl ring and a heteroaryl ring; L₁ is absent;
L₂ is selected from the group consisting of —C(R_{d}R_{c})—, —(CH₂)ₙ—, —NH—, —O—, and —S—;
n is 1, 2 or 3;
Z is a member selected from the group consisting of hydroxy, hydroxyalkyl, R_{g}—O— and Rⱼ—NH—;
R₁ is hydrogen, C₁₋₃ haloalkyl or C₁₋₃ alkyl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

18. A compound according to claim 1 wherein
A is selected from the group consisting of heteroaryl and heteroarylalkyl,
B is selected from the group consisting of an aryl ring and a heteroaryl ring;
D is selected from the group consisting of an aryl ring and a heteroaryl ring; L₁ is selected from the group consisting of —NR_{c}—, —NR_{c}CH₂—, —O—, and —S—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

19. A compound according to claim 1 wherein
A is selected from the group consisting of heteroaryl and heteroarylalkyl,
B is selected from the group consisting of an aryl ring and a heteroaryl ring;
D is selected from the group consisting of an aryl ring and a heteroaryl ring; L₁ is selected from the group consisting of —C(O)—, —C(O)O—, —C(O)NH—, —NR_{c}C(O)—, —NR_{c}C(O)—O—, —NR_{c}S(O)₂—, —OC(O)NH—, —OC(O)—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—— and Rⱼ—NH—;
R₁ is hydrogen, C₁₋₃ haloalkyl or C₁₋₃ alkyl;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

20. A compound according to claim 1 wherein
A is selected from the group consisting of heteroaryl and heteroarylalkyl,
B is selected from the group consisting of an aryl ring and a heteroaryl ring;
D is selected from the group consisting of an aryl ring and a heteroaryl ring; L₁ is —C(RₐR_{b})—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₃ haloalkyl or C₁₋₃ alkyl;
Rₐ and R_{b} are each individually selected from the group consisting of hydrogen, alkyl, haloalkyl, hydroxy or Rₐ and R_{b} taken together with the atom to which they are attached form R_{f}—N═;
R_{c} is selected from the group consisting of hydrogen, alkyl, aryl, haloalkyl and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₃ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(═NH)—, H₂NC(O)alkyl-NHC(O)—, H₂N—O—C(O)—, heteroaryl, heteroarylcarbonyl, heterocycle, and heterocyclecarbonyl.

21. A method of inhibiting ACC, comprising administrating a therapeutically effective amount of a compound of formula (I).

22. A method of inhibiting ACC-1, comprising administering a therapeutically effective amount of a compound of formula (I).

23. The method comprising a compound selected from the group consisting of

24. A method of inhibiting ACC-2, comprising administering a therapeutically effective amount of a compound of formula (I).

25. The method comprising a compound selected from the group consisting of

26. A method of treating metabolic syndrome, comprising administering a therapeutically effective amount of a compound of formula (I).

27. A method of treating type II diabetes, comprising administering a therapeutically effective amount of a compound of formula (I).

28. A method of treating obesity, comprising administering a therapeutically effective amount of a compound of formula (I).

29. A pharmaceutical composition comprising a therapeutically effective amount of a compound of formula (I) in combination with a pharmaceutically suitable carrier.
